(19) United States
(12) Patent Application Publication
(10) Pub. No.: US 2024/0026261 A1
(43) Pub. Date: Jan. 25, 2024

(54) ENGINEERING OF ORGANOID CULTURE FOR ENHANCED ORGANOGENESIS IN A DISH

(71) Applicant: THE TRUSTEES OF THE UNIVERSITY OF PENNSYLVANIA, Philadelphia, PA (US)

(72) Inventors: Dongeun HUH, Villanova, PA (US); Sunghee Estelle PARK, Philadelphia, PA (US)

(21) Appl. No.: 18/255,398

(22) PCT Filed: Dec. 6, 2021
(86) PCT No.: PCT/US2021/072762

§ 371 Date: Jun. 1, 2023

Related U.S. Application Data
(60) Provisional application No. 63/121,684, filed on Dec. 4, 2020.

(51) Int. Cl. C12M 3/00; C12M 1/32; C12M 1/00; C12M 1/12

(52) U.S. Cl. CPC C12M 21/08; C12M 23/12; C12M 23/22; C12M 23/34; C12M 25/14

(57) ABSTRACT

The disclosed subject matter provides techniques for culturing organoids or cells. A device for culturing organoids can include an access port configured to receive a solution, a loading chamber, wherein the access port is located in the loading chamber, and a plurality of culture chambers, wherein the culture chambers are radiated from the loading chamber so that the solution injected into the loading chamber through the access port is distributed into the plurality of culture chambers, wherein the plurality of culture chambers are open to an external environment and comprises a protruding edge at an opening of the plurality of culture chambers.

### CROSS-REFERENCE TO RELATED APPLICATIONS

This application claims the benefit of U.S. Patent Application No. 63/121,684, filed on Dec. 4, 2020, the content of which is hereby incorporated by reference in its entirety.

### STATEMENT REGARDING FEDERALLY SPONSORED RESEARCH

This invention was made with government support under HL127720 awarded by the National Institutes of Health and 1548571 awarded by the National Science Foundation. The government has certain rights in the invention.

### BACKGROUND

Organoids can be used for emulating the complex process of tissue and organ development in vitro. Stem cells in three-dimensional (3D) culture can give rise to self-organizing multicellular structures termed organoids that can resemble the anatomical and functional units of the organ from which they are derived. As organoids can recapitulate the complexity of in vivo physiological systems at the convenience of in vitro cell culture, they can be used for modeling healthy and/or diseased states of various adult organs for biomedical and pharmaceutical applications.

To provide a 3D environment for the organoid culture, certain techniques require embedding stem cells in sessile drops of extracellular matrix (ECM) hydrogels prepared from solubilized basement membrane extracts (e.g., Matrigel). When supplied with culture media containing defined soluble factors that permit proper cell growth and directed differentiation into organ-specific lineages, the 3D environment can induce the differentiating cells to segregate into distinct domains and undergo fate specification, leading to their spontaneous organization into organ-like structures. Despite its utility and versatility, however, these techniques can be limited due to the limited lifespan of organoids. In a typical setup, developing organoids embedded in an ECM hydrogel rely on passive diffusion for nutrient supply and waste removal. This mode of transport effectively supported the organoids through the hydrogel scaffold at their initial stages of development. As the organoids grow and become more metabolically demanding, however, limited diffusion of nutrients and oxygen into the inner regions of the 3D scaffold causes a progressive and significant reduction in the viability of organoids, resulting in the formation of a necrotic core. The rate at which this degenerative process occurs varies depending on the type of organoids, but in most cases, considerable cell death becomes evident within 10 days of culture using the ECM hydrogel. Certain culture techniques can avoid cell death by passaging organoids every 5-7 days. However, such a short duration of each cycle can interrupt the continuous culture of organoids for prolonged periods necessary for their sustained development and maturation into in vivo-like tissue constructs.

To address this problem, researchers have used bioreactors to improve diffusive transport of oxygen and nutrients in 3D culture of organoids. As demonstrated by recent work on cerebral organoids, this approach has proven instrumental for establishing long-term culture to promote continued development and increased maturity of organoids. Implementing this technique in routine laboratory settings, however, is burdened by the need for capital equipment that is mechanically complex and requires specialized knowledge for operation and maintenance. Another drawback is that organoids are cultured in suspension in bioreactors, which makes it challenging to monitor their growth and development during culture. While vascularization of organoids has been suggested as an alternative strategy to improve nutrient supply, the process of generating organoid models with controlled vascular perfusion is prohibitively complex and often requires advanced in vitro systems and specialized techniques that are not easily accessible to non-engineers.

Therefore, there is a need for improved techniques that can be used for uninterrupted and continuous culture of organoids for prolonged periods.

### SUMMARY

The disclosed subject matter provides techniques for culturing organoids and/or cells. An example device for culturing organoids can include an access port configured to receive a solution, a loading chamber, and a plurality of culture chambers. In non-limiting embodiments, the access port can be located in the center of the loading chamber. In some embodiments, the culture chambers can be radiated from the loading chamber so that the solution injected into the loading chamber through the access port can be evenly distributed into the culture chambers. In non-limiting embodiments, the culture chambers can be open to an external environment and include a protruding edge at an opening of the culture chambers.

In certain embodiments, the device can include poly(dimethylsiloxane). In non-limiting embodiments, the device can be optically transparent.

In certain embodiments, the solution can be a hydrogel solution. In non-limiting embodiments, the hydrogel solution can include cells and/or organoids. In some embodiments, the organoid can be a human organoid. In non-limiting embodiments, each culture chamber can include a different type of cells or organoids for co-culturing. In some embodiments, at least about 80% of the organoids in the culture chamber can be viable at day 21 of culturing. In non-limiting embodiments, the growth of the organoids can continue for at least 21 days. In some embodiments, the size of the organoids can increase for at least 21 days. In certain embodiments, the device can reduce the variability in the size of the organoids.

In certain embodiments, each of the culture chambers can have a width and a height ranging from about 100 μm to about 5 cm. In non-limiting embodiments, the protruding edge can be configured to pin a meniscus of the solution at the opening of the culture chambers for filing the entire culture chambers without spillage of the solution through the open-top.

The disclosed subject matter also provides methods for culturing organoids. An example method can include injecting a hydrogel precursor solution including organoids into a loading chamber through an access port, filling a plurality of culture chambers with the hydrogel precursor solution including organoids, solidifying the hydrogel precursor solution to form a hydrogel in the plurality of culture chambers, and providing culture media that is contacted to the hydrogel through the open-top. In non-limiting embodiments, the access port can be located in the center of the loading chamber. In some embodiments, the culture chambers can be radiated from the loading chamber so that the hydrogel precursor solution injected into the loading chamber can be evenly distributed into the culture chambers. In non-limiting embodiments, the culture chambers can be open to an external environment and include a protruding edge at an opening of the culture chambers for preventing spillage of the hydrogel precursor solution through the open-top.

In certain embodiments, the culture media includes soluble factors. In non-limiting embodiments, the soluble factors can include a growth factor, an active agent, or a combination thereof.

In certain embodiments, the method can further include maturing the organoids. In non-limiting embodiments, the method can further include assessing the viability and maturation of the organoids in the plurality of the culture chamber. The plurality culture chamber can be transparent. In some embodiments, the organoid can be a human organoid.

According to an embodiment, the present disclosure relates to a device for culturing organoids, comprising an access port configured to receive a solution, a loading chamber, wherein the access port is located in the loading chamber, and a plurality of culture chambers, wherein the culture chambers are radiated from the loading chamber so that the solution injected into the loading chamber through the access port is distributed into the plurality of culture chambers, wherein the plurality of culture chambers are open to an external environment and comprises a protruding edge at an opening of the plurality of culture chambers.

In an embodiment, the device comprises poly(dimethylsiloxane). In an embodiment, the device is optically transparent. In an embodiment, the access port is located in a center of the loading chamber. In an embodiment, the plurality of culture chambers are symmetrical with respect to rotations about the access port. In an embodiment, the solution injected into the loading chamber through the access port is evenly distributed into the plurality of culture chambers. In an embodiment, the device is configured to contact a culture media from the external environment through the opening of the plurality of culture chambers. In an embodiment, the solution is a hydrogel solution. In an embodiment, the hydrogel solution comprises cells or organoids. In an embodiment, the organoids are human organoids. In an embodiment, each of the culture chambers has a width or a height ranging from about 100 μm to about 5 cm. In an embodiment, each of the culture chambers has a width and a height of about 1 cm. In an embodiment, at least about 80% of the organoids in the culture chamber are viable at day 21 of culturing. In an embodiment, the protruding edge is configured to pin a meniscus of the solution at the opening of the culture chambers, allowing filling of the culture chambers without spillage of the solution through the opening. In an embodiment, each culture chamber comprises a different type of cells or organoids for co-culturing. In an embodiment, growth of the organoids continues for at least about 21 days. In an embodiment, a size of the organoids increases for at least about 21 days. In an embodiment, the device decreases variability in the size of the organoids.

According to an embodiment, the present disclosure relates to a method for culturing organoids, comprising injecting a solution including cells or organoids into a loading chamber through an access port, filling a plurality of culture chambers with the solution including cells or organoids, wherein the culture chambers are radiated from the loading chamber so that the solution injected into the loading chamber is distributed into the plurality of culture chambers, wherein the plurality of culture chambers are open to an external environment and comprises a protruding edge at an opening of the culture chambers for preventing spillage of the solution through the opening, and providing a culture media to the device through the opening of the plurality of culture chambers.

In an embodiment, the culture media comprises soluble factors. In an embodiment, the soluble factors are selected from the group consisting of a growth factor, an active agent, and a combination thereof. In an embodiment, the method further comprises maturing the organoids. In an embodiment, the method further comprises assessing viability and maturation of the organoids in the plurality of culture chambers.

The disclosed subject matter will be further described below.

### BRIEF DESCRIPTION OF THE DRAWINGS

FIGS. 1A-1G provide photographs and diagrams of an example system for culturing an organoid in accordance with the disclosed subject matter. The scale bar of the micrograph of FIG. 1B-1D is 500 μm. The scale bar of the top image of FIG. 1F is 5 mm and the scale bar of the bottom image of FIG. 1F is 3 mm.

FIGS. 2A-2S provide graphs and confocal images showing the long-term culture of intestinal organoids using the disclosed system in accordance with the disclosed subject matter. The scale bar of FIGS. 2A-2C, 2F, 2G, and 2K is 100 μm.

FIGS. 3A-3O provide graphs and confocal images showing the maturation of intestinal organoids in accordance with the disclosed subject matter. The scale bar of FIGS. 3C, 3D, and 3J is 100 μm. The scale bar of FIGS. 3I, 3L, and 3M is 10 μm.

FIGS. 4A-4K provide graphs and images showing the functional characterization of intestinal organoids in the disclosed system in accordance with the disclosed subject matter. The scale bar of FIGS. 4E-4F is 100 μm.

FIGS. 5A-5G provide graphs and images showing co-culture in the disclosed system in accordance with the disclosed subject matter. The scale bar of FIG. 5A and FIG. 5C and the left side of FIG. 5D is 5 mm.

FIGS. 6A-6R provide graphs and images showing an example model of intestinal fibrosis for drug testing in accordance with the disclosed subject matter.

FIG. 7 provides diagrams showing an example fabrication of the disclosed system in accordance with the disclosed subject matter.

FIG. 8 provides diagrams and confocal images showing the comparison of bud length between the hydrogel culture system and the disclosed system in accordance with the disclosed subject matter.

FIG. 9 provides a graph showing cellular compositions of intestinal organoids in the disclosed system in accordance with the disclosed subject matter.

FIG. 10 provides a confocal image showing the continuous growth of organoids in the disclosed system in accordance with the disclosed subject matter. The scale bar of FIG. 10 is 200 μm.

FIGS. 11A-11C provide diagrams and images showing the human intestinal organoid culture using the disclosed system in accordance with the disclosed subject matter.

FIGS. 12A-12Q provide diagrams and images showing prolonged culture of human intestinal organoids using the disclosed system in accordance with the disclosed subject matter. FIG. 12A—Human enteroids derived from human adult intestinal stem cells cultured in OCTOPUS and Matrigel drop for 5 days. Scale bars, 100 μm. FIG. 12B and FIG. 12C—During 14 day-culture, enteroids in OCTOPUS become larger and develop crypt/villus-like structures (top), which is in contrast to arrested growth and decreased viability in Matrigel drop culture (bottom). Scale bars, 100 μm. FIG. 12D and FIG. 12E—Quantification of organoid viability (12D) and size (12E). FIG. 12F and FIG. 12G—Representative images of H&E stained enteroid sections in OCTOPUS and Matrigel drop at days 7 (12D) and 14 (12E). Scale bars, 20 μm. FIG. 12H and FIG. 12I—Quantification of bud number (12H) and length (12I). FIG. 12J—Growth of human enteroids in OCTOPUS over 21 days. Scale bars, 50 μm. FIG. 12K and FIG. 12P—Immunofluorescence and mRNA analysis of Ki67+ proliferative cells in the crypt domain (12K, 12L) and differentiated intestinal epithelial cells on the villus surface, including KRT20+ absorptive enterocytes (12M, 12N) and MUC2+ goblet cells (o, p) at days 7 and 14. Scale bars, 10 μm. Data are presented as mean±SEM. *P<0.05, **P<0.01, and ***P<0.001 (n≥3).sd

FIGS. 13A-13S provide diagrams and images showing single-cell RNA sequencing of human enteroids using the disclosed system in accordance with the disclosed subject matter. FIG. 12A—UMAP projection of 12 clusters representing distinct stem and intestinal epithelial cell populations in human enteroids produced by 7 days of culture in OCTOPUS. FIG. 12B through FIG. 12D—UMAP plots showing the expression of representative canonical genes specific to absorptive enterocytes (FIG. 12B), goblet cells (FIG. 12C), and stem cells (FIG. 12D). FIG. 12E, FIG. 12F—UMAP projection of cell clusters in human enteroids after 7-day culture in Matrigel drop (e) and 14 days of uninterrupted culture in OCTOPUS (FIG. 12F). FIG. 12G—Quantification of cellular compositions in human enteroids. Where available, the percentage of each cell type measured in the native human intestine is shown with a dashed line. FIG. 12F—Violin plots comparing the expression of select cell-type-specific maturation markers between Matrigel drop culture and OCTOPUS. FIG. 12I—Pseudotime trajectories (top) and branching plot (bottom) of intestinal stem cell differentiation into secretory and absorptive cell populations in human enteroids cultured in OCTOPUS for 14 days. FIG. 12J—Comparison of the fraction of differentiated epithelial cell types in OCTOPUS and Matrigel drop culture. *P<0.05, **P<0.01, and ***P<0.001.

FIGS. 14A-14X provide diagrams and images showing organoid-based model of human IBD in the disclosed system in accordance with the disclosed subject matter. FIG. 14A—Adult stem cells isolated from the intestine of IBD patients are used to form enteroids in OCTOPUS. FIG. 14B, FIG. 14C—Morphology of IBD patient-derived and normal enteroids in OCTOPUS after 14-day culture visualized by immunofluorescence (14B) and H&E staining (c). Scale bars, 100 μm (14B) and 5 μm (14C). FIG. 14D, FIG. 14E—Quantification of enteroid size (14D) and the number of buds (14E) at days 7 and 14. FIG. 14F, FIG. 14G—Comparison of cell proliferation (Ki67) and apoptosis (caspase-3 and annexin V) in IBD and normal enteroids. Scale bars, 10 μm. FIG. 14H—Confocal micrographs and quantification of ZO-1 expression by differentiated epithelial cells on the villus domain of enteroids. Scale bars, 10 μm. FIG. 14I—Visualization of 4-kDa dextran-FITC diffusion into the organoid lumen (L) to show epithelial permeability in the IBD enteroids. Scale bars, 50 μm. FIG. 14J, FIG. 14K—UMAP projection of transcriptomically distinct cell populations (14J) and quantification of their proportions (14K) in IBD and normal enteroids after 14-day culture in OCTOPUS. FIG. 14L—Comparison of IBD-associated genes. FIG. 14M—Heatmap showing the mean expression of transcription factors in IBD enteroids relative to that in normal enteroids. FIG. 14N—Upregulation of lncRNA genes in the IBD enteroids occurs mostly in Paneth cells shown with dashed lines in the UMAP plots. FIG. 14O—The intestinal epithelium supported by the underlying stroma is modeled in OCTOPUS by mixed co-culture of human enteroids and primary human intestinal fibroblasts in the same hydrogel scaffold. FIG. 14P—Confocal micrograph of the co-culture construct at day 14. Scale bar, 100 μm. FIG. 14Q—Immunofluorescence micrographs of localized regions surrounding the enteroids after 14 days of culture. Scale bars, 25 μm. FIG. 14R—Quantification of FN production and fibroblast proliferation in the stroma. FIG. 14S—Quantification of cytokines released by day 14 enteroids. Data are presented as mean±SEM. *P<0.05, **P<0.01, and ***P<(n≥3). FIG. 14U—When cultured in Matrigel drops, IBD enteroids show properly polarized epithelial cells (top right) that resemble those in the epithelium of normal enteroids. In comparison to IBD enteroids in OCTOPUS, they also retain the structural integrity of the epithelium as visualized by ZO-1 expression (bottom right). Scale bars, 5 μm.

FIGS. 15A-150 provide diagrams and images showing microengineering of vascularized human enteroids in the disclosed system in accordance with the disclosed subject matter. FIG. 15A—Photo of OCTOPUS-EVO devices in a standard 12-well cell culture plate. FIG. 15B—Device architecture of OCTOPUS-EVO. FIG. 15C, FIG. 15D—Sequential steps of microfluidic 3D culture necessary for generating self-assembled and perfusable blood vessels while supporting self-organization of stem cells into organoids in the same hydrogel scaffold. FIG. 15E—Micrographs demonstrating the concurrent development of human enteroids and microvasculature over the course of 12-day culture. Scale bars, 200 μm. f. Perfusability of the microengineered vascular network visualized by the flow of 1-μm fluorescent beads. Scale bars, 100 μm. FIG. 15G—Comparison of organoid size between vascularized and non-vascularized constructs. FIG. 15H—Construction of vascularized, perfusable human IBD enteroids in OCTOPUS-EVO. Scale bars, 100 μm. i. Quantification of vascular density and vessel diameter. FIG. 15J, FIG. 15K—Pro-inflammatory phenotype of the vascularized IBD model demonstrated by endothelial expression of ICAM-1 (15J) and increased production of inflammatory mediators (15K). Scale bars, 50 μm. FIG. 15L—Micrograph of IBD enteroids perfused with peripheral blood monocytes. Scale bar, 200 μm. FIG. 15M, FIG. 15N—Confocal microscopy (15M) and quantification (15N) of sequential steps of monocyte recruitment to IBD enteroids. Scale bars, 50 μm. Data are presented as mean±SEM. *P<0.05, **P<0.01, and ***P<0.001 (n≥3).

It is to be understood that both the foregoing general description and the following detailed description are exemplary and are intended to provide further explanation of the disclosed subject matter.

### DETAILED DESCRIPTION

The disclosed subject matter provides techniques for culturing cells and/or organoids. The disclosed techniques can provide enhanced organogenesis and extended life span of the cells or organoids. The disclosed techniques can also enhance the maturity of the cells and organoids. The disclosed techniques can also permit the enlargement of the cells and organoids. The disclosed techniques can also reduce the variability of the cells and organoids.

Unless otherwise defined, all technical and scientific terms used herein have the same meaning as commonly understood by one of ordinary skill in the art. In case of conflict, the present document, including definitions, will control. Certain methods and materials are described below, although methods and materials similar or equivalent to those described herein can be used in the practice or testing of the presently disclosed subject matter. All publications, patent applications, patents, and other references mentioned herein are incorporated by reference in their entirety. The materials, methods, and examples disclosed herein are illustrative only and not intended to be limiting.

As used herein, the term “organoid” generally describes a 3D multicellular in vitro tissue construct that mimics its corresponding in vivo organ such that it can be used to study aspects of that organ. As used herein, the term “organoid” describes any geometry of self-organized three-dimensional tissue culture. In certain instances, the term “organoid” may be further defined as comprising stem cells and/or somatic cells.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, up to 10%, up to 5%, and up to 1% of a given value. Alternatively, particularly with respect to biological systems or processes, the term can mean within an order of magnitude, within 5-fold, and within 2-fold, of a value.

The present disclosure introduces a facile, scalable engineering approach to enable long-term development and maturation of organoids. Method described herein have redesigned the three-dimensional configuration of conventional organoid culture to develop a platform that converts single injections of stem cell suspensions to radial arrays of organoids that can be maintained for extended periods. Using human and mouse stem cells, accelerated production of intestinal organoids and their sustained development for over 4 weeks without the need for passaging is demonstrated. Compared to conventional techniques, long-term culture in the disclosed device enhances the formation of the crypt-villus structures and significantly increases the functional maturity of the intestinal epithelium. Further, vascularized, perfusable human enteroids can be assembled in a microengineered device and used to model the recruitment of innate immune cells to the diseased intestinal epithelium in IBD. The disclosed system, methods, and device may provide an immediately deployable platform to engineer more realistic organ-like structures in a dish.

The present disclosure describes a simple, immediately deployable strategy based on rethinking the design of conventional 3D culture of organoids. The methods described herein utilize an advanced platform capable of reconfiguring the geometry of 3D culture scaffolds to generate open arrays of organoids that eliminate the problem of limited and non-uniform diffusion inherent in bulk hydrogel. The systems described herein can be manufactured as simple and ready-to-use culture inserts of different sizes and shapes that can be used in standard cell culture plates without any modification of established protocols and workflow.

A proof-of-concept of the present disclosure is demonstrated by continuous growth and development of mouse intestinal organoids during extended periods of uninterrupted culture. Resulting intestinal tissue constructs exhibit structural and functional maturity not achievable in conventional culture. The utility of the approach can be further demonstrated by the production and prolonged maintenance of human intestinal organoids and in-depth analysis of their significantly enhanced maturity using single-cell RNA sequencing (scRNA-seq). Finally, the advanced capabilities of the technology can be demonstrated by establishing i) an organotypic model of human inflammatory bowel disease (IBD) using patient-derived enteroids and ii) microengineered organoids integrated with perfusable vasculature for in vitro modeling of immune-epithelial interaction in IBD.

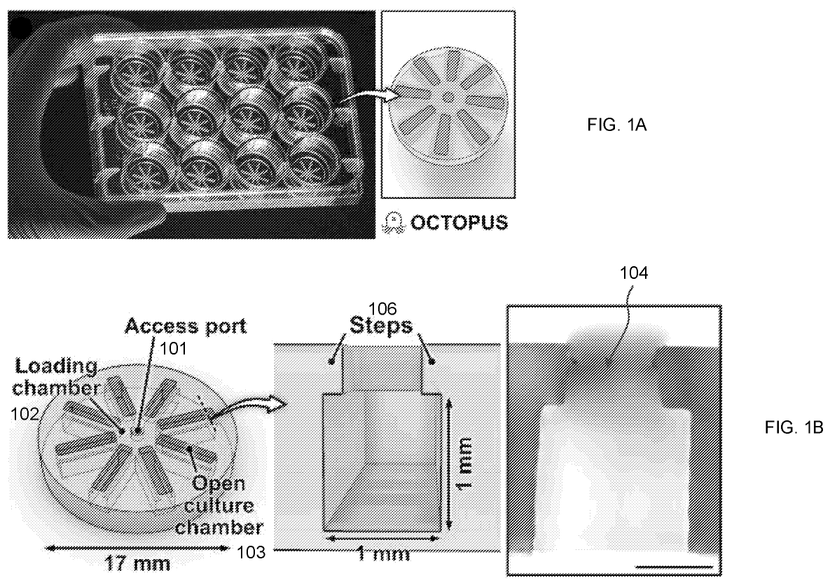

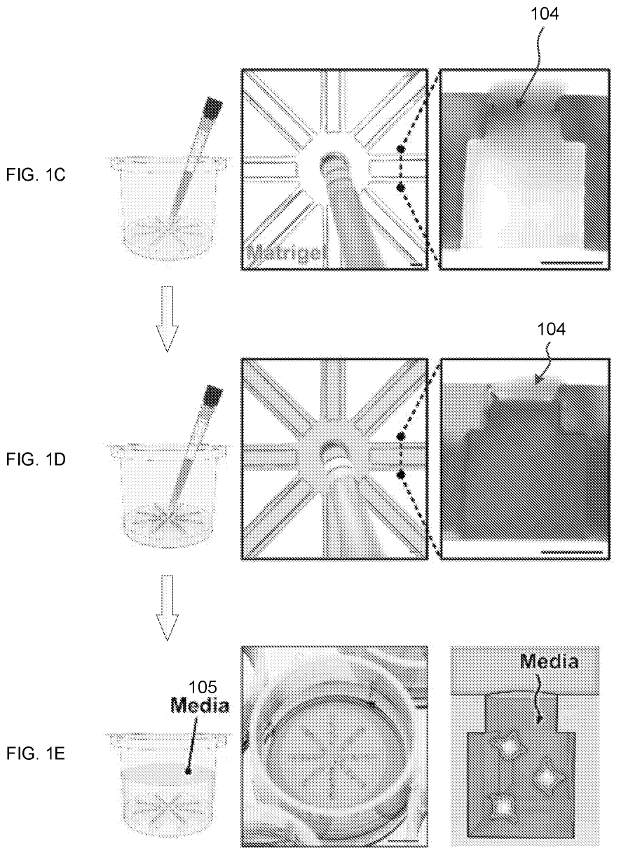

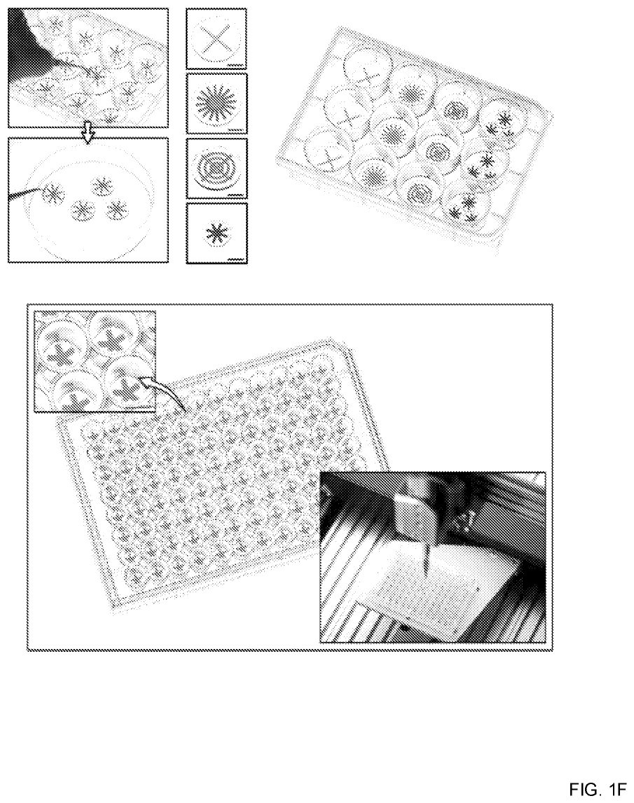

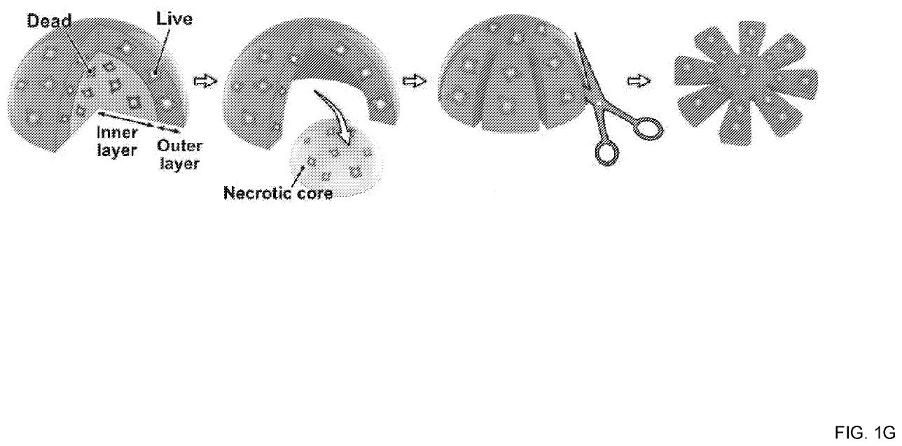

As background, it should be appreciated that central to the challenge of long-term culture in traditional organoid models is the formation of areas of increased cell death due to diffusion limitations in the inner regions of sessile hydrogel drops. In essence, the approach described herein can be conceptualized as i) removing this necrotic core from the hydrogel scaffold while keeping the outer layer in which organoids remain viable and ii) radially segmenting this layer and spreading it out to form a planar array of organoids, as shown in FIG. 1G. By substantially decreasing the thickness of the culture scaffold, this array is designed to permit unrestricted diffusion and replenishment of nutrients, oxygen, and other soluble factors, which makes it possible to create a more uniform and sustainable biochemical microenvironment conducive to long-term culture.

For practical implementation of this idea, a disc-shaped 3D culture device that enables the production and maintenance of radially arranged organoid arrays in standard cell culture plates was created (FIG. 1A). Introduced briefly, this device, termed OCTOPUS (Organoid Culture-based Three-dimensional Organogenesis Platform with Unrestricted Supply of soluble signals), consists of one or more organoid culture chambers radiating from a central loading chamber. In an embodiment, the OCTOPUS may include an open access port at the center of the radiating one or more organoid culture chambers. In an embodiment, each culture chamber may have cross-sectional dimensions of 1 mm (height)×1 mm (width). Importantly, the culture chambers are open to the external environment and contain a microscopic part protruding from the edges of the opening (FIG. 1B). In this system, stem cells suspended in an ECM hydrogel precursor solution are manually pipetted into the central chamber through the access port (FIG. 1C). Thanks to the geometric symmetry of the device design, the injected mixture is equally distributed to the culture chambers (FIG. 1D). During this process, surface tension acts to pin the meniscus of the liquid at the protruding edges of the chamber ceiling (FIG. 1D), allowing the injected solution to advance and fill the entire chamber without spillage through the open-top. After gelation, the culture medium is added to the device-containing well to provide nutrient supply to the embedded cells through the exposed hydrogel surface (FIG. 1E). Establishing a 3D culture in OCTOPUS only requires these two simple pipetting procedures without having to make any changes to the standard procedure used for conventional organoid culture.

In addition to procedural simplicity and convenience, OCTOPUS enables new capabilities that render the method advantageous over conventional techniques. The design of OCTOPUS as a removable culture insert makes this system easily transferable (FIG. 1F), facilitating the handling, manipulation, and analysis of organoid culture models established in the device. The approach also offers design flexibility. The key parameters that define the architecture of OCTOPUS are readily adjustable during device fabrication to vary the number, size, shape, and connectivity of culture chambers (FIG. 1F), which provides a means to control the volume and spatial organization of organoid-containing 3D tissue constructs generated in the system. Similarly, the overall size and shape of OCTOPUS can easily be changed to create devices that are compatible with standard culture plates with different well sizes and formats. This flexibility is of particular importance as it allows for scalability. As demonstrated in FIG. 1F, OCTOPUS can be deployed as a culture platform in a 96-well format coupled with automated liquid handling systems to scale up the production of organoid models for applications that require significantly increased experimental throughput.

Briefly introduced above, the system, methods, and device of the present disclosure will now be described in more detail below with reference to the Drawings.

In certain embodiments, the disclosed subject matter provides a device for culturing cells, organoids, or tissue explants. As shown in FIG. 1B, an exemplary device can include an access port 101, a loading chamber 102, and at least one culture chamber 103 (e.g., 8 culture chambers 103 in FIG. 1B). The access port 101 can be located in the center of the loading chamber 102, and the at least one culture chamber 103 can radiate from the loading chamber 102.

In certain embodiments, the loading chamber 102 can be configured to receive a solution through the access port 101. For example, a solution can be pipetted into the loading chamber 102 through the access port 101. In non-limiting embodiments, the access port 101 can be located in the center of the loading chamber 102. In some embodiments, the device can include more than one loading chamber 102 for co-culturing different types of cells and organoids. In certain embodiments, the loading chamber 102 can include poly(dimethylsiloxane) (PDMS). In certain embodiments, the loading chamber 102 can include polystyrene, thermoplastic, glass, metal, paper, or combinations thereof.

In certain embodiments, the loading chamber 102 can have a diameter ranging from about 2 mm to about 10 mm. In non-limiting embodiments, the access port 101 can have a diameter ranging from about 0.5 mm to about 3 mm.

In certain embodiments, the culture chamber 103 can be radiated from the loading chamber 102 so that the solution injected into the loading chamber 102 through the access port 101 can be evenly distributed into the at least one culture chamber 103. In non-limiting embodiments, multiple culture chambers 103 can be radiated from the loading chamber 102. In some embodiments, the disclosed device can include more than one loading chamber 102, and each loading chamber can be connected to one or more culture chambers for the co-culturing platform. Each culture chamber 103 can include a different type of cells or organoids, while they can be exposed to the same culture media. Each culture chamber 103 can include a different type of extracellular matrix. In certain embodiments, each culture chamber 103 can contain independently accessible flow channels. In certain embodiments, the culture chamber 103 can include PDMS. In certain embodiments, the culture chamber 103 can include polystyrene. In certain embodiments, the culture chamber 103 can include thermoplastics. In certain embodiments, the culture chamber 103 can include glass. In certain embodiments, the culture chamber 103 can include metals. In certain embodiments, the culture chamber 103 can include paper.

In some embodiments, the culture chamber 103 can have a width ranging from about 100 μm to about 50 mm. In non-limiting embodiments, the culture chamber 103 can have a height ranging from about 100 μm to about 5 cm. In some embodiments, the shape and size of the culture chamber 103 or can be modified depending on the purposes of the disclosed device (e.g., co-culture, target cells, and target organoids). In some embodiments, the culture chamber 103 can have a width and/or height of about 100 μm to about 5,000,000 μm. In some embodiments, the culture chamber 103 can have a width and/or height of at least about 100 μm. In some embodiments, the culture chamber 103 can have a width and/or height of at most about 5,000,000 μm. In some embodiments, the culture chamber 103 can have a width and/or height of about 100 μm to about 1,000 μm, about 100 μm to about 10,000 μm, about 100 μm to about 50,000 μm, about 100 μm to about 100,000 μm, about 100 μm to about 500,000 μm, about 100 μm to about 1,000,000 μm, about 100 μm to about 5,000,000 μm, about 1,000 μm to about 10,000 μm, about 1,000 μm to about 50,000 μm, about 1,000 μm to about 100,000 μm, about 1,000 μm to about 500,000 μm, about 1,000 μm to about 1,000,000 μm, about 1,000 μm to about 5,000,000 μm, about 10,000 μm to about 50,000 μm, about 10,000 μm to about 100,000 μm, about 10,000 μm to about 500,000 μm, about 10,000 μm to about 1,000,000 μm, about 10,000 μm to about 5,000,000 μm, about 50,000 μm to about 100,000 μm, about 50,000 μm to about 500,000 μm, about 50,000 μm to about 1,000,000 μm, about 50,000 μm to about 5,000,000 μm, about 100,000 μm to about 500,000 μm, about 100,000 μm to about 1,000,000 μm, about 100,000 μm to about 5,000,000 μm, about 500,000 μm to about 1,000,000 μm, about 500,000 μm to about 5,000,000 μm, or about 1,000,000 μm to about 5,000,000 μm. In some embodiments, the culture chamber 103 can have a width and/or height of about 100 μm, about 1,000 μm, about 10,000 μm, about 50,000 μm, about 100,000 μm, about 500,000 μm, about 1,000,000 μm, or about 5,000,000 μm.

In certain embodiments, the culture chambers can be open to an external environment. As shown in FIG. 1B through FIG. 1E, cells, organoids, hydrogels, or combinations thereof in the culture chamber can be supplied with nutrients and/or culture media through opening 104 of the culture chamber 103. For example, the device can be submerged in culture media 105 with nutrients, and the nutrients can be uniformly diffused into the entire hydrogel in the culture chamber. For example, when supplied with culture media containing the soluble signals, the hydrogel scaffold allows rapid media diffusion throughout the 3D culture chamber, providing nutrient supply to the cells and/or organoids within 30 minutes.

In certain embodiments, the culture media can include nutrients, soluble factors, growth factors, active agents, or combinations thereof. For example, when supplied with culture media containing the soluble/growth factors that permit proper cell growth and directed differentiation into organ-specific lineages, the cells and/or organoids in the 3D culture chamber can be differentiated into organ-like structures. In certain embodiments, the culture media can include soluble/growth factors such as R-spondin ligand, Noggin, bone morphogenetic protein (BMP), epithelial growth factor (EGF), fibroblast growth factor (FGF), B-27, N-2, BSA, ascorbic acid, MTG, Glutamax, CHIR99021, rhKGF, 8BrcAMP, IBMX, DMH-1, A83-01, hydrocortisone, and heparin. In non-limiting embodiments, the culture media can include a target active agent for screening drugs. For example, intestinal stem cells can be seeded into the culture chamber 103 and treated with anti-fibrotic drugs (e.g., Pirfenidone and/or Nintedanib) at pre-determined concentrations for testing the effects of the drugs on the fibrotic phenotype. In non-limiting embodiments, the active agent can include chemicals, toxins, nanomaterials, bacteria, viruses, nucleic acids, peptides, or combinations thereof.

In non-limiting embodiments, the culture chamber 103 can include a protruding edge 106, or step 106, at the opening 104 of the culture chamber 103. The protruding edge 106 can be configured to pin a meniscus of the injected solution at the opening-top of the culture chamber 103 for filing the entire culture chamber 103 without spillage of the solution through the open-top.

In certain embodiments, the culture chamber 103 can be coated for enhancing the adhesion of a gel and/or a cell to the inner surface of the culture chamber 103. For example, each culture chamber 103 can be filled with a dopamine hydrochloride solution at room temperature (RT) to form a surface coating for enhanced adhesion of a hydrogel.

In certain embodiments, the disclosed device can include PDMS. In certain embodiments, the loading chamber can include polystyrene. In non-limiting embodiments, the device can be optically transparent. For example, cells or organoids embedded in a hydrogel located in the disclosed device can be observed through microscopic techniques (e.g., bright-field, confocal, fluorescence, electron, atomic force, and laser scanning microscopy) without removing the hydrogel from the disclosed device. In certain embodiments, the device can have a size ranging from about 1 mm to about 50 cm.

In certain embodiments, the solution injected into the loading chamber can be a hydrogel solution. For example, the hydrogel solution can be an extracellular matrix (ECM) precursor solution, which can be solidified (i.e., gelation) after in the culture chamber, providing 3D culture environments. In non-limiting embodiments, the solution can include cells, organoids, or tissue explants. The cells can be any cells that can be cultured in vitro. For example and not limitation, the cells can be stem cells, goblet cells, endothelial cells, epithelial cells, mesenchymal cells, neural cells, muscle cells, progenitor cells, immune cells, endocrine cells, or combinations thereof. The organoids can be any organoids that can be cultured in vitro. For example and not limitation, the organoids can include human organoids, mouse organoids, intestinal organoids, liver organoids, lung organoids, nascent organoids, or combinations thereof.

In certain embodiments, the organoids cultured in the disclosed device can have an extended life span. For example, The organoids cultured in the disclosed system can survive up to about 3 weeks without passaging. In non-limiting embodiments, at least about 80% of the organoids in the culture chamber can be viable at day 5, 10, 14, and 21 of culture.

In certain embodiments, the disclosed device can provide improved morphological and functional maturation of the organoids. The long-term culture capabilities of the disclosed device can be leveraged to increase the maturity of intestinal organoids. For example, during culture (e.g., for about 7 days), the flat epithelium can be folded into finger-like protrusions (e.g., villi) and have extended budding, which can be longer than the villi cultured without the disclosed device. In addition to morphological development, the disclosed device can provide improved functional maturation of organoids. For example, the villi cultured in the disclosed device can express higher functional markers (e.g., peptide transporter 1, sodium-glucose linked transporter 1 (SGLT1), and glucose transporter 2 (GLUT2)) than the villi cultured without the disclosed device.

In certain embodiments, the disclosed subject matter provides methods for culturing organoids. An example method can include injecting a hydrogel precursor solution including organoids into a loading chamber through an access port, filling a plurality of culture chambers with the hydrogel precursor solution including organoids, solidifying the hydrogel precursor solution to form a hydrogel in the plurality of culture chambers, and providing culture media that is contacted to the hydrogel through the open-top. For example, to form organoids in the disclosed device, the organoid/hydrogel mixture can be generated by mixing the hydrogel precursor solution with a pellet of organoids in a complete organoid growth medium. Using a pre-wetted pipet tip, about 100 μl of the organoid/Matrigel mixture can be injected into the disclosed device through the access port. The mixture can be evenly distributed through the culture chamber without spillage of the mixture solution. For example, each culture chamber can have the same volume of the mixture after being injected through the access port. The disclosed device can be incubated for gelation of the hydrogel precursor solution. Pre-warmed organoid growth media can be added to each culture chamber for long term culture.

In certain embodiments, the method can further include assessing the viability and maturation of the organoids in the plurality of the culture chamber through the transparent device. For example, viability and maturation of the organoids can be assessed through microscopic techniques (e.g., bright-field, confocal, fluorescence, electron, atomic force, and laser scanning microscopy) and biochemical analyses (e.g., ELISA).

According to an embodiment, a device according to the above can be fabricated by casting PDMS prepolymer against micropatterned three-dimensional printed molds using standard soft lithography techniques. For example, PDMS (Sylgard 184, Dow Corning, USA) monomer base can be mixed with a curing agent (10:1, w/w) and poured onto 3D printed molds (Protolabs, USA). The casted molds can be vacuum degassed in a desiccation chamber for 30 minutes, after which the PDMS can be oven cured overnight at 65° C. to produce devices containing organoid culture chambers, as described in FIG. 1A through FIG. 1F. The cured PDMS can be removed from the molds, stamped against a thin layer of uncured PDMS (spin-coated onto a flat wafer at 1500 rpm for 5 minutes), and then sealed against a thin slab of cured PDMS that formed the bottom layer of the device. Each assembled OCTOPUS can be baked at 65° C. to fully cure the stamped PDMS adhesive layer and then placed in a 24-well plate until use.

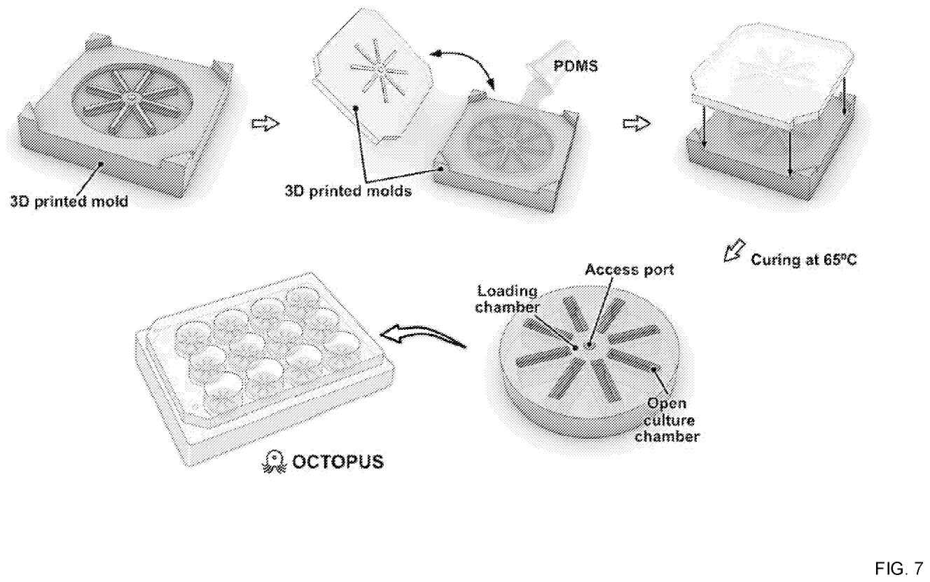

FIG. 7 provides a flow diagram of the above-described device fabrication method. To fabricate the device, degassed PDMS prepolymer can be dispensed into a 3D printed mold patterned with protruding features of organoid culture chambers, loading chamber, an access port. The mold can then be covered with another 3D printed mold that contains matching positive relief patterns to generate openings for the organoid culture chambers and access port. After curing PDMS at 65° C. for 2 hours, the PDMS slab can be peeled off of the molds. Finally, the fabricated devices can be placed in a standard multi-well plate, as shown in FIG. 1A.

### Examples

### Results

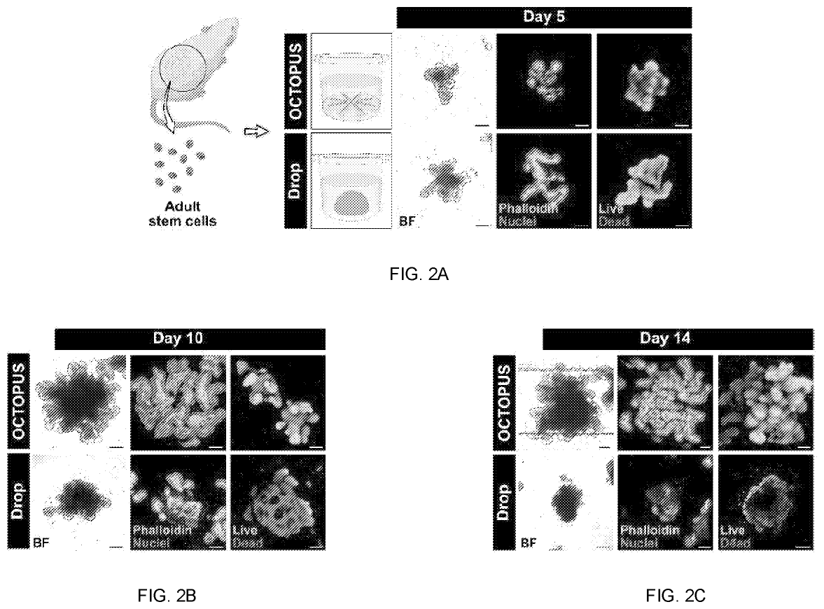

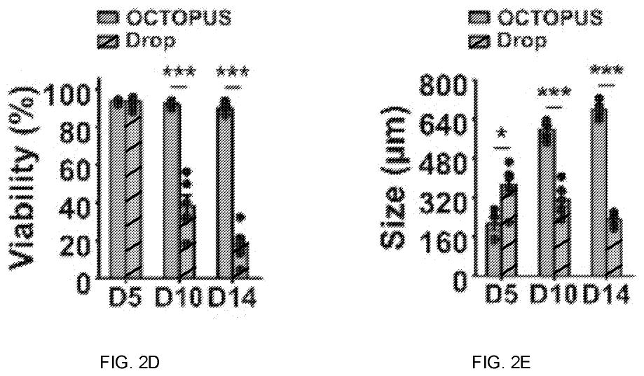

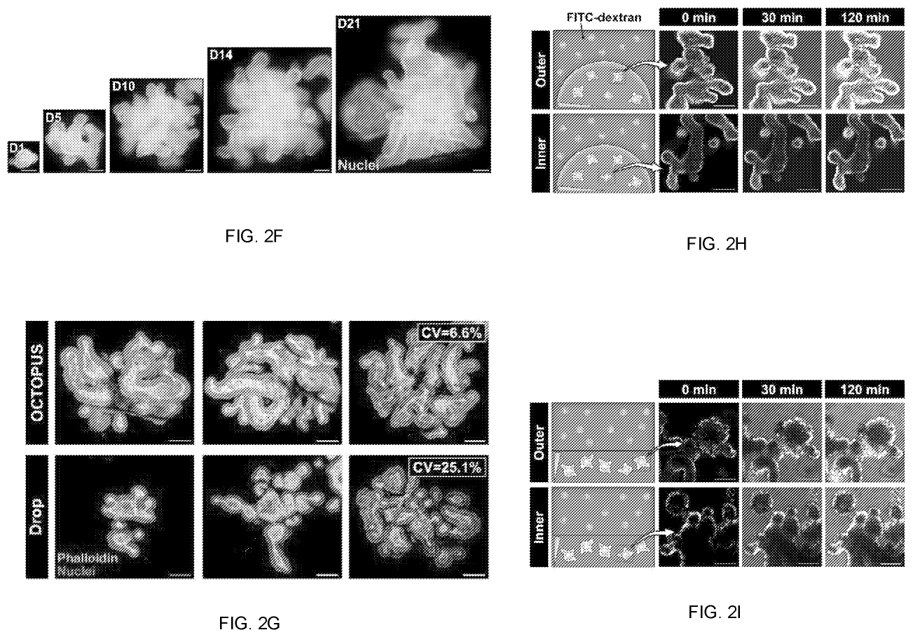

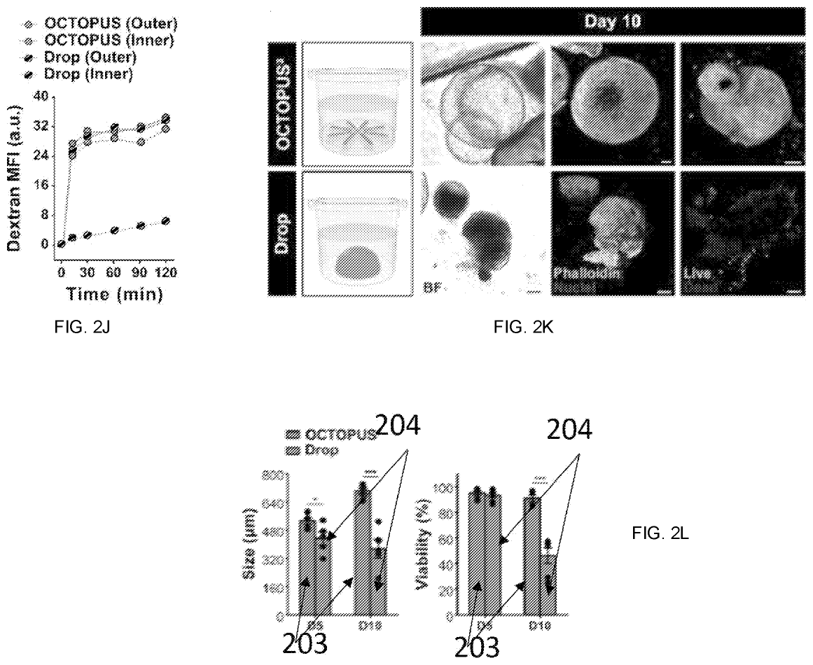

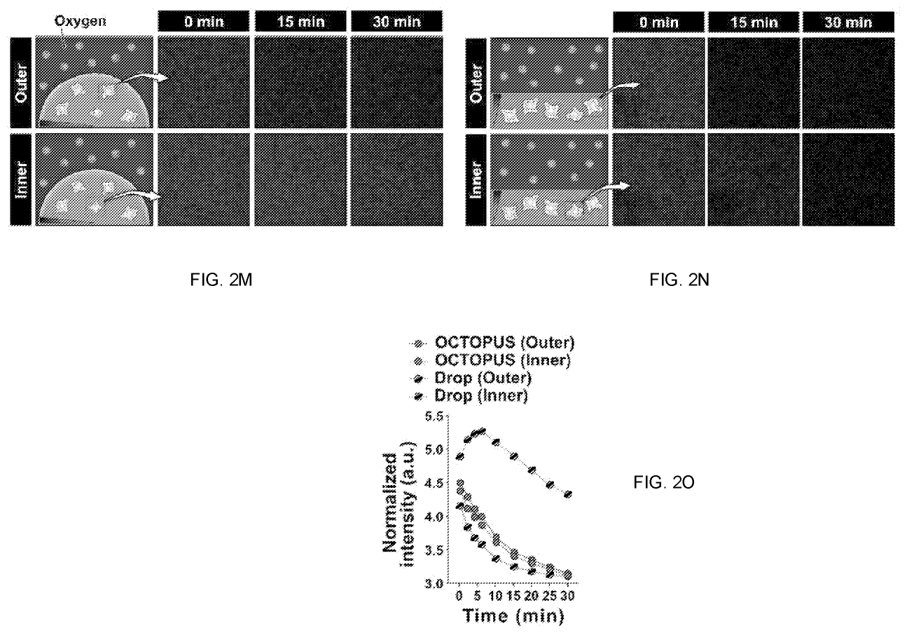

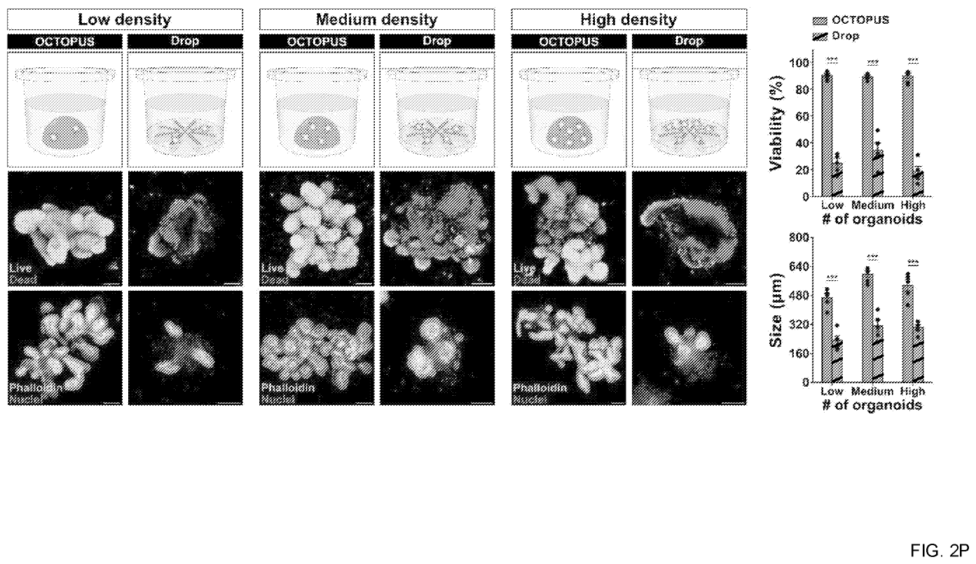

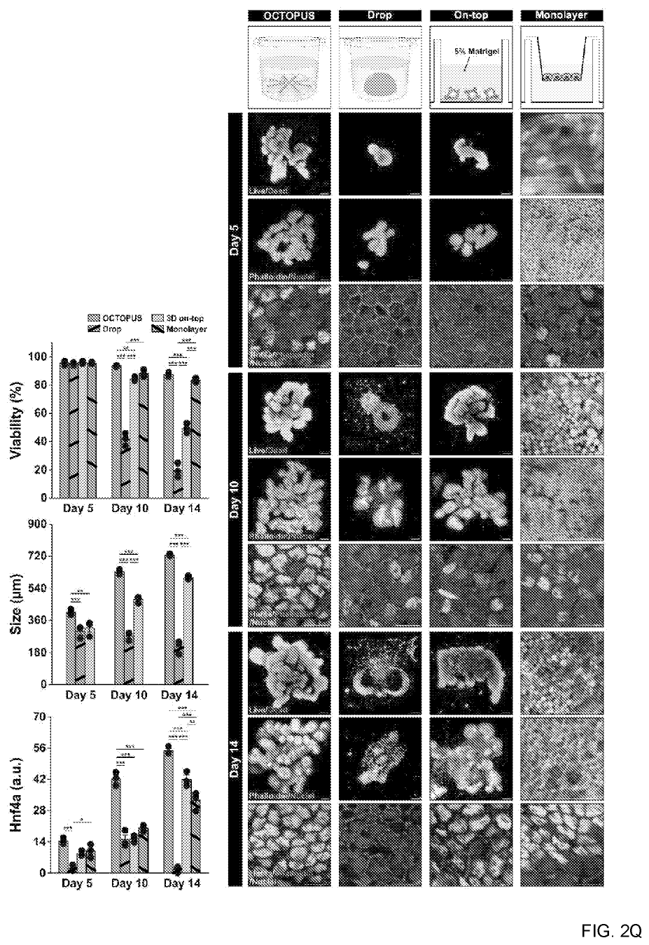

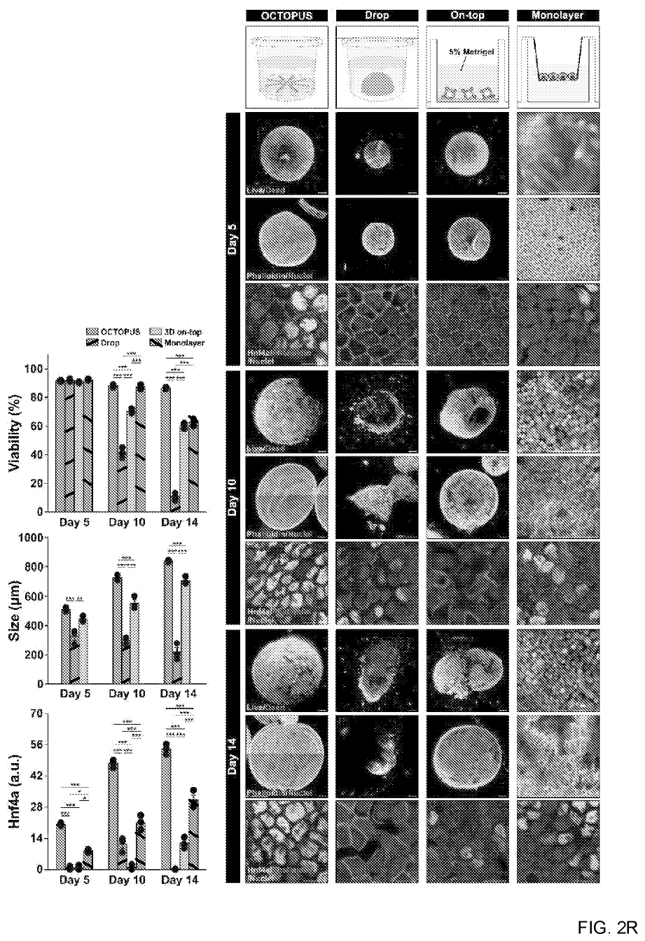

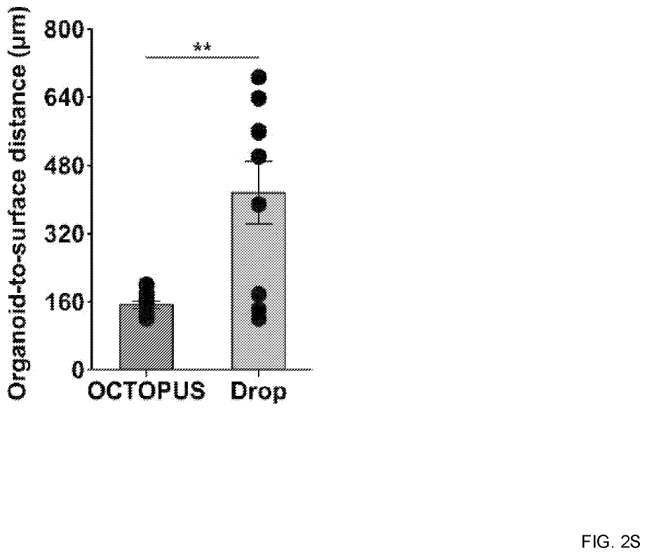

The disclosed device can extend the lifespan of organoids. To simulate the most common settings of standard organoid culture, small intestinal organoids derived from commercially available mouse adult stem cells and protocols were selected as a model system. FIG. 2A shows mouse intestinal adult stem cells in Matrigel self-assemble into intestinal organoids in both OCTOPUS and drop culture. During culture, the cells embedded in Matrigel arrays of OCTOPUS underwent the process of self organization over a period of 5 days in a manner described by the manufacturer's protocol and previous studies to form intestinal organoids identified by their crypt-villus structures (top row in FIG. 2A). These organoids exhibited similar viability and morphological characteristics to those formed in sessile drops of Matrigel with commonly used sizes (˜3 mm in radius) using the same protocol (bottom row in FIG. 2A; FIG. 2D). However, when the culture period was extended beyond 5-7 days, which is the maximum recommended duration of continuous culture before passaging, considerable differences between the two groups were noticed. The organoids in the OCTOPUS continued to grow and form buds (top row in FIG. 2B and FIG. 2C) without a measurable loss of viability, as quantified in FIG. 2D, resulting in a 3.2-fold increase in their size after 14 days of culture, as shown in FIG. 2E. In contrast, a large fraction (˜65%) of organoids maintained in Matrigel drops developed necrosis by day 10 (bottom row in FIG. 2B; quantified in FIG. 2D), which was further exacerbated over time to yield more than 80% reduction in viability at the end of 14-day culture (bottom row in FIG. 2C; quantified in FIG. 2D). This significant cell death led to collapse and disintegration of the organoids as evidenced by their morphology (bottom row in FIG. 2C) and a progressive decrease in size (FIG. 2E). The higher cell viability and increased organoid growth in OCTOPUS were observed regardless of initial cell seeding density, as shown in FIG. 2P. OCTOPUS also showed a superior ability to support long-term viability and development of organoids (FIG. 2Q and FIG. 2R) when compared to other conventional techniques modified from the Matrigel drop culture method, such as 3D ‘on-top’ culture and monolayer culture of organoid-derived cells. FIG. 2F shows a continuous enlargement of intestinal organoids in OCTOPUS over 21 days. FIG. 2G shows that OCTOPUS reduces variability in the size of organoids, as evidenced by the substantially smaller coefficient of variation. The images show organoids at day 14. Notably, the more supportive environment of OCTOPUS permitted prolonged culture and continuous growth of organoids for over 3 weeks without passaging, as shown in FIG. 2F, representing a more than 3-fold increase in their lifespan per culture cycle as compared to the typical duration of conventional intestinal organoid culture in Matrigel drops (5-7 days). Moreover, the enhanced longevity of culture in OCTOPUS was accompanied by significantly reduced variability in the size of developing organoids (FIG. 2G).

In an attempt to provide further insight into the observed differences, temporal profiles of 70-kDa FITC-dextran permeation into organoid-containing Matrigel scaffolds to simulate passive diffusion of soluble factors in the disclosed device was measured. For spatial analysis, these measurements were taken at two locations representing the inner and outer regions of the scaffold. FIG. 2H and FIG. 2I show 70 kDa FITC-dextran diffusion into the inner and outer regions of the hydrogel scaffolds in Matrigel drop (FIG. 2H) and OCTOPUS (FIG. 2I). The organoids in the inner and outer regions were located 600 μm (OCTOPUS)/2400 μm (Matrigel drop) and 400 μm (both groups) from the hydrogel surface, respectively. FIG. 2J shows temporal profiles of mean fluorescence intensity (MFI) due to dextran diffusion. FIG. 2K shows the formation and extended culture of mouse liver organoids in OCTOPUS and conventional drop culture. FIG. 2L shows quantification of the size and viability of liver organoids in OCTOPUS 203 and Drop 204.

These results suggest that the 3D culture environment in the device can allow for unrestricted and spatially uniform diffusive transport of soluble factors. In Matrigel sessile drops, unrestricted transport of dextran into the outer layer was evident from the rapid increase in fluorescence intensity within 30 minutes, which was in contrast to limited dye penetration into the core of the scaffold (FIG. 2H, FIG. 2J). Importantly, this significant spatial variability was not observed in OCTOPUS, in which dextran diffusion occurred at almost the same rate across the entire hydrogel construct to reach the saturation level in less than 30 minutes (FIG. 2I, FIG. 2J). In this case, the temporal profiles of fluorescence intensity in the inner and outer regions of the scaffold closely matched those measured in the surface layer of hydrogel drops (FIG. 2J).

Consistent with these findings, oxygen penetration into the Matrigel scaffold in OCTOPUS occurred in a rapid and uniform manner, resulting in oxygen saturation through the entire thickness of the construct within 30 minutes (FIG. 2N, FIG. 2O). This pattern of diffusion was different from that in Matrigel drops that clearly showed the development of oxygen gradients across the scaffold and the presence of a hypoxic core that remained throughout the culture period (FIG. 2M, FIG. 2O).

These results verify the design principle of OCTOPUS and suggest that long-term viability and sustained growth of organoids with reduced size variability shown in the disclosed system may be attributed to unrestricted and spatially uniform diffusive transport of nutrients, growth factors, and oxygen, which is achieved by decreasing the distance between organoids and the Matrigel surface (FIG. 2S). While this demonstration was based on the use of intestinal organoids, the results also showed the feasibility of extending the same approach to other types of organoids. For example, OCTOPUS offered similar beneficial effects on the culture of liver organoids as illustrated by their continuous growth over prolonged periods without a loss of viability (FIG. 2K). Taken together, the data demonstrate that OCTOPUS provides significant added value beyond conventional culture techniques by extending the lifespan of organoids.

Limited lifespan of organoids in conventional culture hampers their ability to reach later stages of development and acquire a more mature phenotype. The long-term of OCTOPUS can be leveraged to increase the maturity of intestinal organoids in the model system.

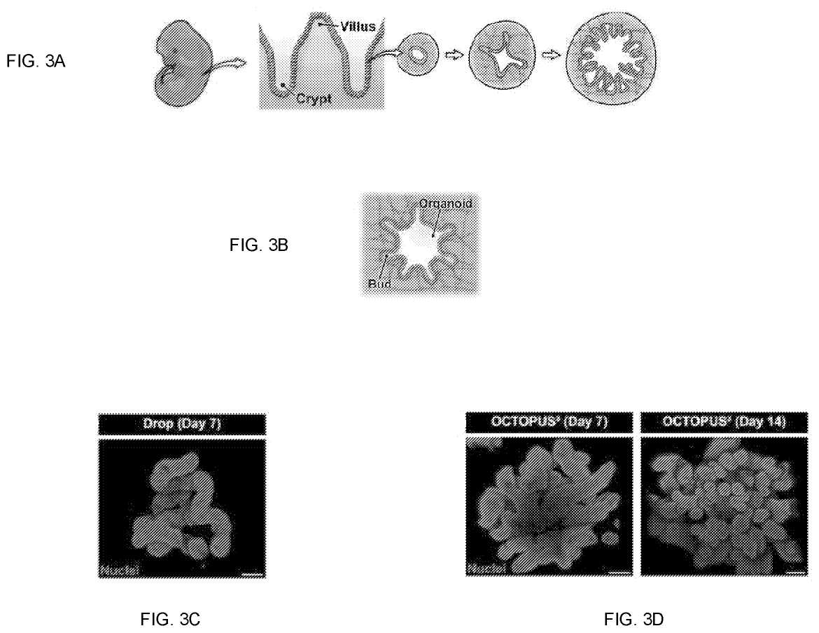

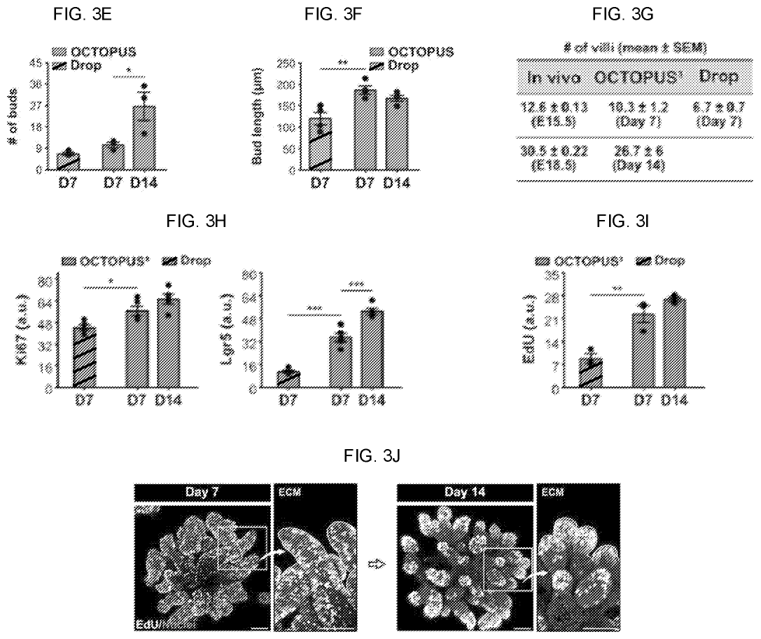

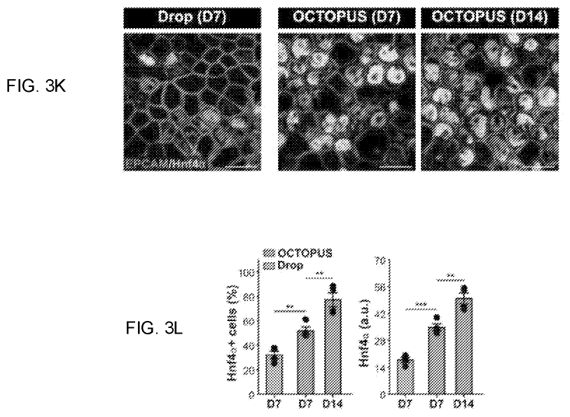

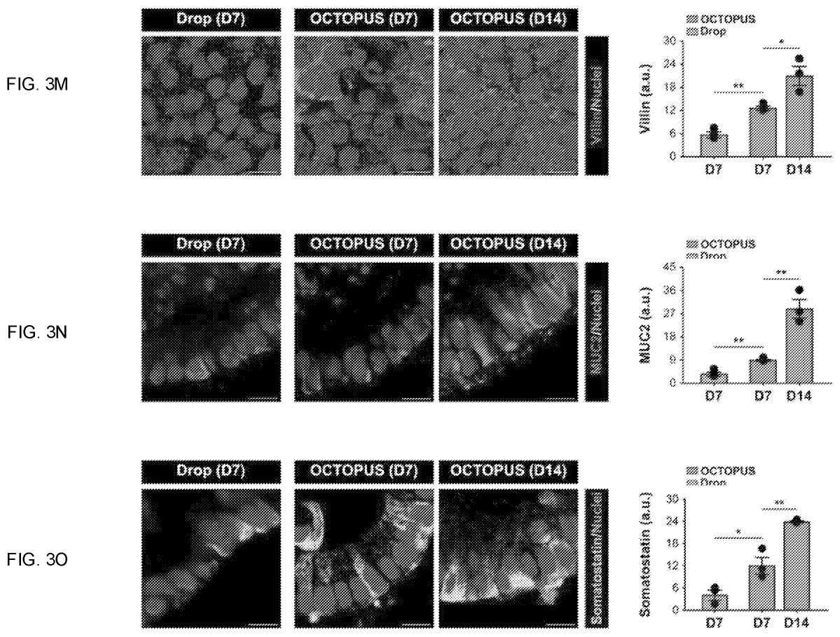

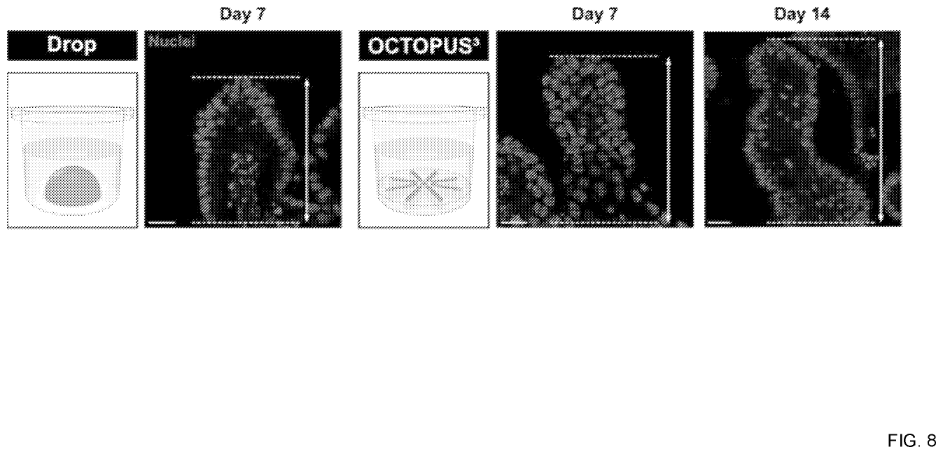

During embryogenesis, the flat epithelium of the developing gut tube begins to fold into finger-like protrusions called the villi, which are separated by deep invaginations known as the crypts (FIG. 3A). FIG. 3A shows the formation of the villus-crypt architecture during intestinal development in vivo. As development continues, the number of villi increases to make the crypt-villus structures more pronounced and drastically expand the epithelial surface area available for nutrient absorption. Focusing on this critical process of morphogenesis, the morphology of intestinal organoids was examined by measuring the number and length of buds that correspond to the crypt-like domains of organoids (FIG. 3B). FIG. 3B shows bud formation used as a metric for analyzing morphological development of intestinal organoids in 3D culture. During culture for 7 days, the formation of folded structures in developing organoids was clearly visible in both Matrigel drops and OCTOPUS, but the extent of budding appeared to be greater in OCTOPUS (FIG. 3C to 3E). FIG. 3C and FIG. 3D show confocal micrographs of organoids in Matrigel drop (FIG. 3C) and OCTOPUS (FIG. 3D). Organoid budding is more pronounced in OCTOPUS. Importantly, the device allowed these organoids to continue their development beyond day 7 to form roughly three times as many buds by day 14 (FIG. 3D, FIG. 3E). FIG. 3E and FIG. 3F show quantification of bud number (FIG. 3E) and length (FIG. 3F) cultured in OCTOPUS and Drop. In contrast, organoids in hydrogel drops ceased to bud and rapidly lost their viability after 7 days of culture (data not shown). The analysis also showed significantly elongated buds in OCTOPUS-generated organoids (FIG. 3F and FIG. 8). Of note is that the numbers of buds from OCTOPUS at days 7 and 14 were good approximations of those measured in mouse embryos at E15.5 and E18.5, respectively (FIG. 3G). FIG. 3G shows in vitro-in vivo comparison of the number of villi at different stages of development. Similarly, the average length of buds in these organoids (167.25 μm) was comparable to in vivo measurements (130.95 μm).

Results also showed higher expression of stem cell markers (Lgr5, Ki67) (FIG. 3H) and more robust immunostaining of EdU (FIG. 3I) when organoids were generated in the device. FIG. 3H and FIG. 3I show that organoids in OCTOPUS show elevated expression of intestinal stem cell markers (Ki67 and Lgr5) and more active cell proliferation as illustrated by increased immunofluorescence of EdU. This finding corroborates the observed difference in organoid budding (FIG. 3C to FIG. 3F) because the formation and elongation of the crypt-villus structures during organoid development require the expansion of stem/progenitor cells and their active proliferation. Interestingly, the EdU+ proliferative cells in device culture were distributed throughout the entire organoid construct on day 7 (FIG. 3J). FIG. 3J provides confocal micrographs showing the spatial distribution of EdU+ cells (white) in OCTOPUS-generated organoids. The lines in the close-up images outline organoid buds. With the progression of culture, however, these cells were observed to localize at the tip of the buds (FIG. 3J), recapitulating the well-documented restriction of cell proliferation to the crypt domains after villi formation during intestinal development in vivo.

In addition to morphological development, how the 3D culture environment in OCTOPUS affects the emergence of organ-specific tissues in intestinal organoids was assessed. Stem and progenitor cells in the embryonic intestine give rise to a specialized epithelium on the villi that contain absorptive and secretory cells critical for nutrient absorption and other important physiological functions of the intestine. Indeed, this process of epithelial differentiation and maturation occurred in both conventional and OCTOPUS organoid models as evidenced by the expression of hepatocyte nuclear factor 4α (Hnf4α), which is a transcription factor that plays an essential role in intestinal maturation during embryogenesis. Organoids in Matrigel drops, however, were seen with considerably lower expression even at the point of their maximum maturation (day 7) (FIG. 3K, FIG. 3L). FIG. 3K shows that organoids developing in OCTOPUS exhibit increased expression of Hnf4α, a marker of mature intestinal epithelial cells, compared to the control group in Matrigel drop. FIG. 3L shows quantification of the fraction of Hnf4α+ cells and the level of Hnf4α expression. Immunofluorescence of Hnf4α was normalized with respect to the number of cells. In OCTOPUS, the already-higher expression of Hnf4α increased further over the course of 14-day culture, leading to approximately 1.5- and 1.4-fold increases in the fraction of Hnf4α+ cells and the level of expression on a per-cell basis, respectively (FIG. 3K, FIG. 3L).

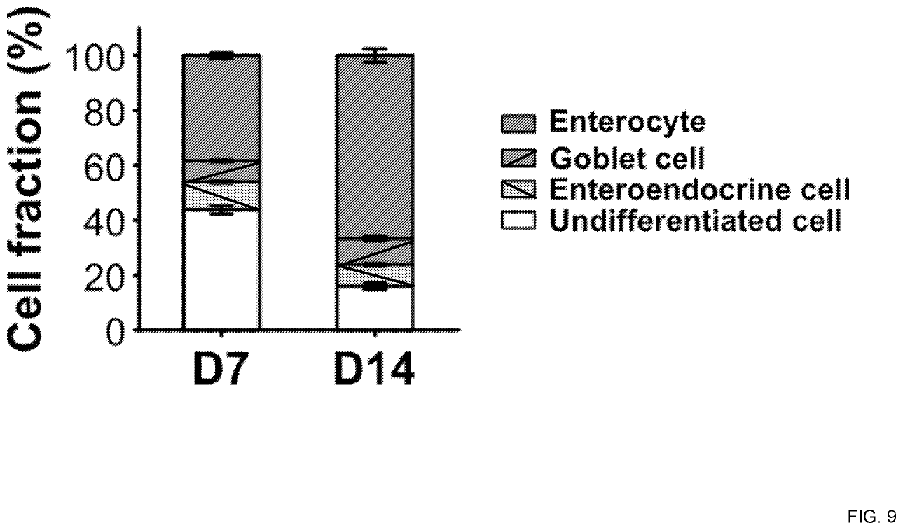

Importantly, the enhanced maturation of organoids in the device was further supported by analysis of cell type-specific markers. At day 7, for example, the expression of villin—a terminal differentiation marker of absorptive enterocytes—was significantly upregulated in OCTOPUS compared to hydrogel drops (FIG. 3M), and this phenotype was further promoted by prolonged culture (FIG. 3M). Similar trends were observed in the induction of makers specific to mucus-producing goblet cells (MUC2) (FIG. 3N) and enteroendocrine cells (somatostatin) responsible for hormone secretion (FIG. 3O). FIGS. 3M-3O shows visualization and quantification of differentiation markers specific to enterocytes (villin, FIG. 3M), goblet cells (MUC2, FIG. 3N), and enteroendocrine cells (somatostatin, FIG. 3O). Interestingly, the number of enterocytes on the villi emerging in the developing organoids was significantly greater than that of the other cell types (FIG. 9), reproducing the highest abundance of these cells in the intestinal epithelium in vivo. These findings suggest that OCTOPUS enables the development of organoids in a more accelerated and sustained manner, allowing them to reach higher levels of morphological and cellular maturity than in certain conventional 3D culture.

In the next phase, whether the enhanced morphogenesis and tissue maturity demonstrated in the device contribute to the functional maturation of intestinal organoids was assessed. Given that the primary function of the intestine is nutrient absorption, key molecular transporters that regulate the absorptive function of the intestinal epithelium, including i) peptide transporter 1 (PEPT1) responsible for intestinal uptake of peptides and ii) sugar transporters including sodium-glucose linked transporter 1 (SGLT1) and glucose transporter 2 (GLUT2) that mediate the absorption of monosaccharides were measured.

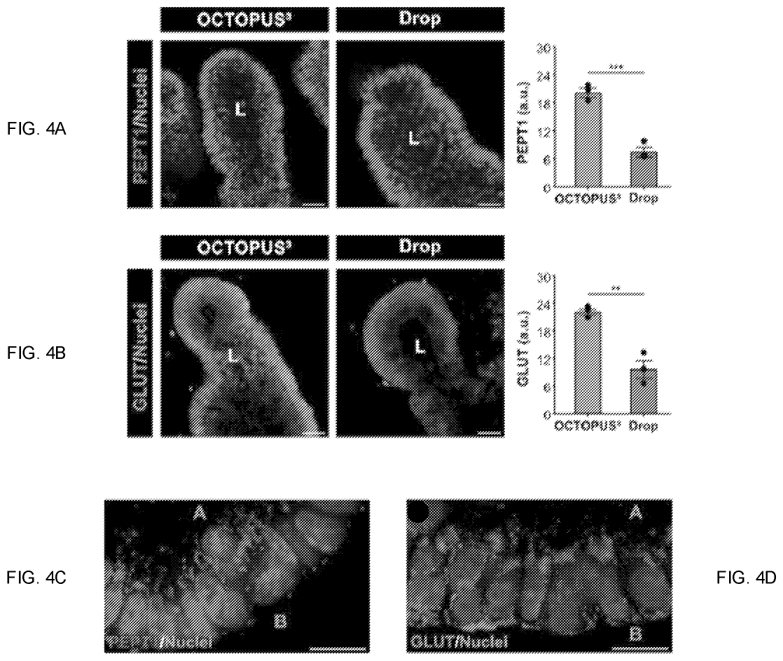

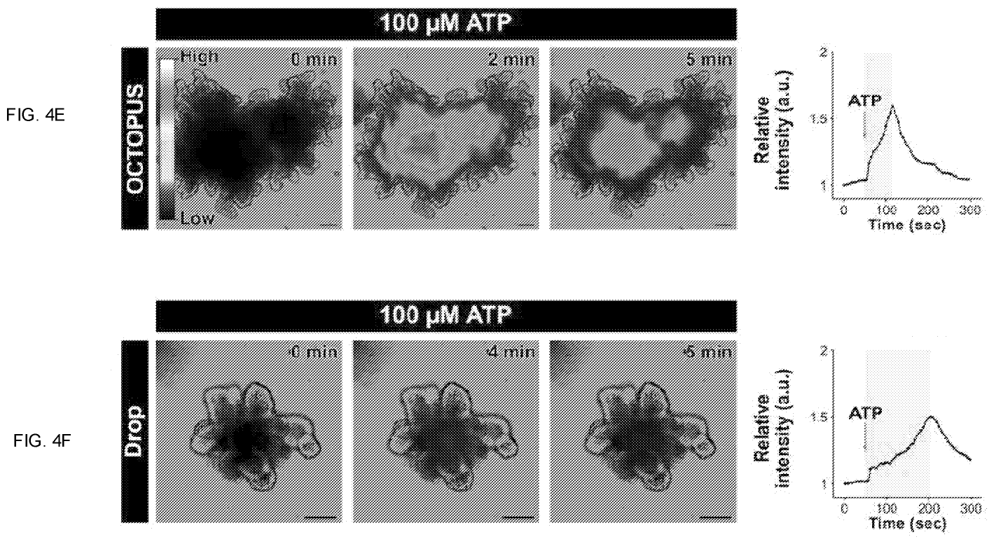

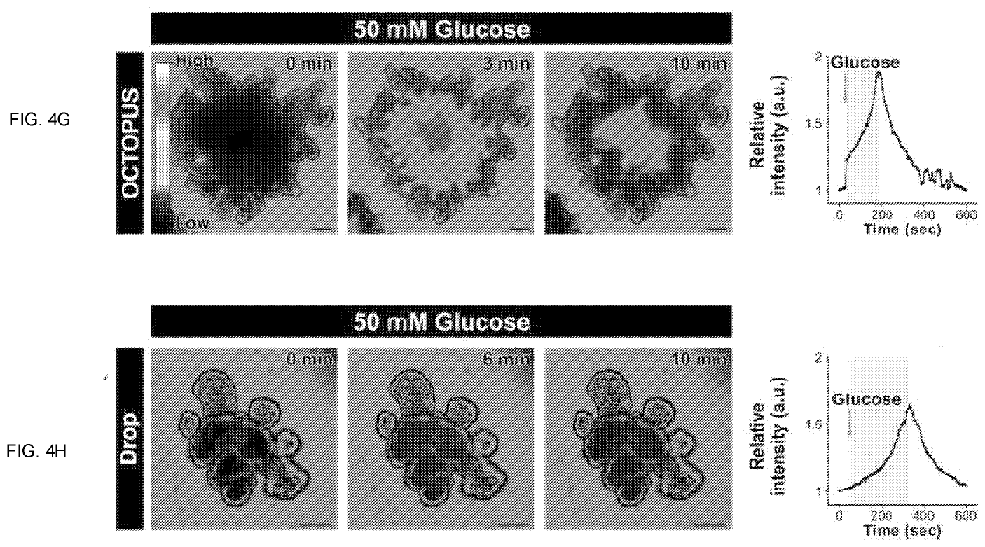

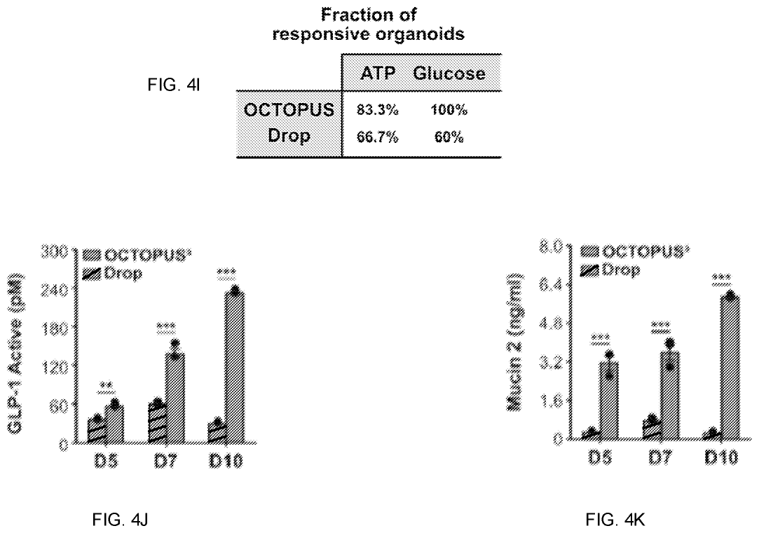

In this analysis, organoids in hydrogel drops at the maximum duration of culture (7 days) were compared to those maintained in OCTOPUS for 14 days to examine the contribution of extended culture. Regardless of the culture platform, immunostaining clearly showed the presence of the transporters on the villi, but the expression of these functional markers was significantly elevated in OCTOPUS (FIG. 4A, FIG. 4B). In the device-generated organoids, PEPT1 was localized to the apical surface of the villi without detectable fluorescence on the basolateral side (FIG. 4C), which is reminiscent of its polarized expression on the brush border membrane of the native intestinal epithelium. The sugar transporters were found on both the apical and basal surfaces (FIG. 4D), capturing the spatial distribution of SGLT1 (apical) and GLUT2 (basal and apical).

For further functional characterization, live-cell imaging techniques were used to visualize intracellular calcium signaling, which has been shown to regulate the activity of the intestinal nutrient transporters detected in the model. To assess the level of intracellular calcium, the organoids were labeled with a fluorescent calcium indicator dye (Fluo-4) and monitored their fluorescence in real-time. Given the multicellular complexity of organoids, the mean fluorescence intensity was measured from representative organoids selected for analysis. Upon treatment with 100 μM ATP, organoids in the device increased their fluorescence by approximately 1.6-fold within 60 seconds, which was followed by a gradual decrease to the baseline level (FIG. 4E). The response of the conventional model to the same stimulation was noticeably slower and occurred to a lesser extent (FIG. 4F). Similarly, when 50 mM D-glucose was used as a more physiologically relevant stimulant, organoid culture in OCTOPUS exhibited calcium responses in a much more rapid and substantial manner than was observed in hydrogel drops under the same treatment conditions (FIG. 4G, FIG. 4H). Comparison between these two groups also revealed that a larger fraction of organoids responded to ATP and glucose in OCTOPUS (FIG. 4I). It was noted that the increase in intracellular calcium measured in the device during glucose treatment was greater than that induced by ATP stimulation (FIG. 4E, FIG. 4G).

The results have shown that intracellular calcium signaling also plays an essential role in the secretion of digestive hormones in response to increased nutrients in the intestinal lumen, which is another important physiological function of the intestine. Based on this evidence, hormone secretion in the disclosed organoid models was assessed as a measure of functional maturation. Enzyme-linked immunosorbent assay (ELISA) of conditioned media was performed to measure the glucose-induced release of glucagon-like peptide 1 (GLP-1), an incretin hormone secreted by enteroendocrine L cells of the intestinal epithelium that enhances glucose-stimulated insulin release from pancreatic β-cells. The ELISA data showed the release of the biologically active form of GLP-1 by the cultured intestinal organoids in response to glucose included in the culture media. Notably, for all three-time points of analysis (days 5, 7, and 10), the hormone was secreted in significantly larger amounts in OCTOPUS than was measured in the conventional model (FIG. 4J). The difference between the two groups was accentuated over time as the organoids in the device continued to develop and mature, giving rise to more than 7-fold higher concentrations of GLP-1 in OCTOPUS after 10 days of culture (FIG. 4J). Although not relevant to hormone secretion, the ELISA analysis also detected MUC2, an intestine-specific glycoprotein that is secreted by goblet cells to form a protective mucus layer on the epithelial surface. The release of MUC2 followed similar trends to those found in the secretion of GLP-1 (FIG. 4K), demonstrating the ability of OCTOPUS to promote the induction and maturation of this secretory phenotype that plays a central role in the barrier function of the intestine.

While organoids have the inherent capacity to reproduce the multicellular complexity of their in vivo counterparts, it remains a significant challenge to emulate the integrated higher-level structure and function of native organs in conventional organoid culture. To meet this challenge, efforts are being made to develop new methods for increasing the cellular heterogeneity of current organoid models and recapitulating biological crosstalk beyond the cellular level of organization to model tissue-tissue and multiorgan interactions. Inspired by this emerging body of work, the possibility of using OCTOPUS to create co-culture models that combine organoids with their associated tissues in 3D culture was assessed.

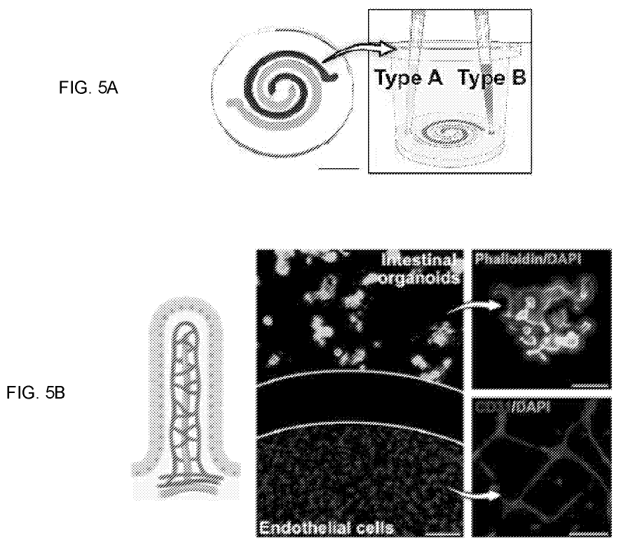

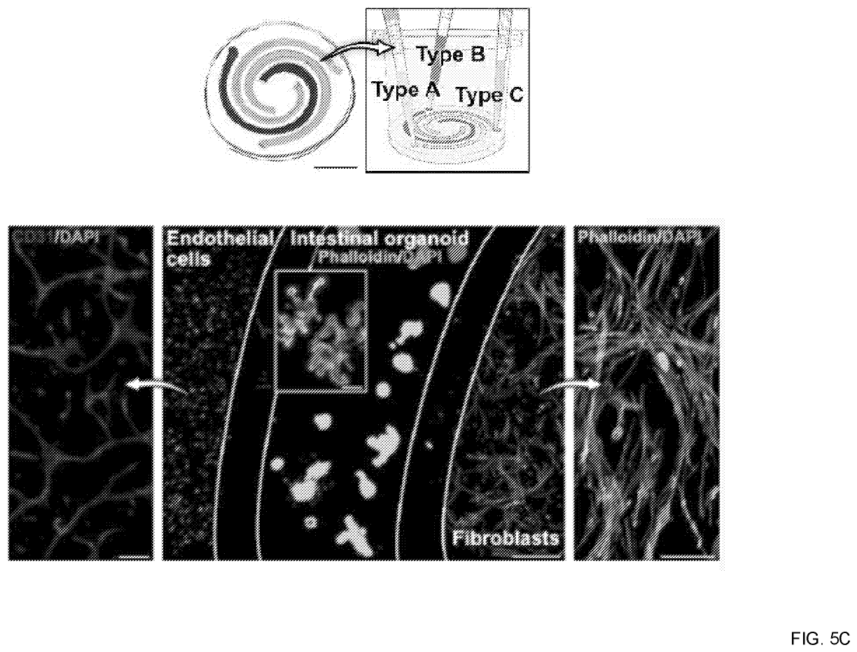

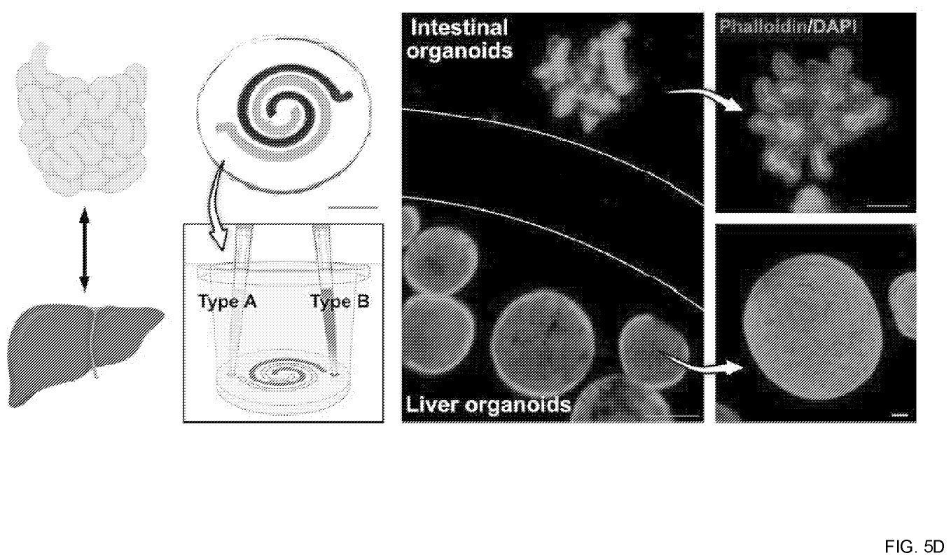

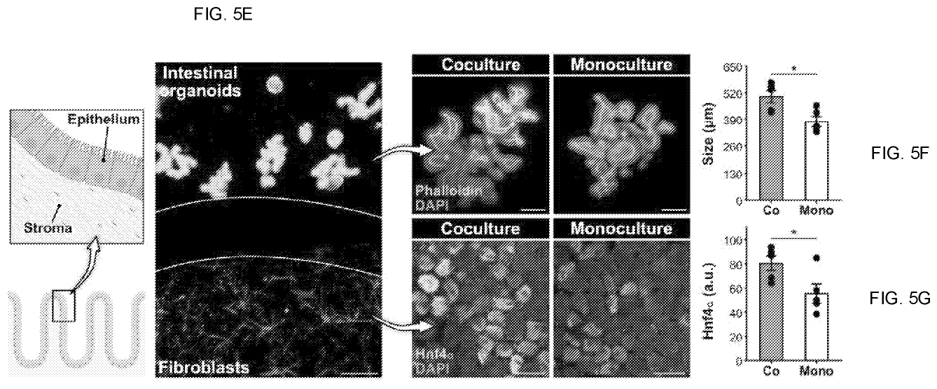

First, the design of OCTOPUS was engineered to incorporate a pair of open spiral culture chambers with individually accessible injection ports (FIG. 5A). In this configuration, the chambers can be filled with different cell types to generate two juxtaposed tissue constructs that can be maintained in the same soluble environment. To demonstrate this approach, a co-culture of small intestinal organoids with vascular endothelial cells embedded in Matrigel (FIG. 5B) was established. This tissue pair was chosen to approximate the intestinal epithelium and the microvasculature in the underlying stroma. The co-culture condition did not interfere with the self-organization of stem cells and allowed them to grow into intestinal organoids with the typical crypt-villus microarchitecture (FIG. 5B). Concurrent to this process, the endothelial cells in the other chamber self-assembled into a 3D network of interconnected endothelial tubes within 5 days of culture (FIG. 5B), mimicking the process of de novo blood vessel formation during development. The resulting vascular network and intestinal organoids were maintained stably over prolonged periods (>10 days).

The dual-chamber design could easily be modified during device fabrication to accommodate a greater number of tissue types. This was demonstrated by increasing the number of chambers to create a tri-culture system that consisted of intestinal organoids and two neighboring 3D constructs containing intestinal fibroblasts and blood vessels (FIG. 3C). OCTOPUS also permitted the incorporation of two or more different types of organoids into a single device to represent multiple organs, as shown by the co-culture of small intestinal organoids with liver organoids (FIG. 5D). In the common soluble environment optimized for co-development of these organoids, the stem cells seeded into two separate compartments formed their respective organ-like constructs following the same timeline of development as monoculture (FIG. 5D). The device supported longterm culture of this organoid pair over two weeks without a loss of viability and structural integrity.

Importantly, analysis of the co-culture models indicated significant effects of non-parenchymal tissues on the development of organoids. For example, when a co-culture of small intestinal organoids with primary intestinal fibroblasts was established (FIG. 5E), the average size of organoids in this system was larger than that in monoculture (FIG. 5F). The co-culture organoids displayed significantly elevated (1.5×) levels of Hnf4α expression when compared to their monoculture counterparts (FIG. 5G), demonstrating the benefit of recapitulating the epithelial-stromal interactions for the growth and maturation of intestinal organoids.

Recognizing that in vitro modeling of complex disease is emerging as the primary focus of organoid research, the potential application of OCTOPUS for this active area of investigation was assessed. With the goal of exploiting the long-term culture capabilities of the disclosed system, a model intestinal fibrosis was established as a representative example of pathophysiological conditions caused by prolonged disease processes that cannot be easily recapitulated in conventional organoid models due to their limited lifespan. Fibrosis is a common complication of intestinal diseases such as inflammatory bowel disease and gastrointestinal cancer. The intrinsic ability of the intestine to repair wounds and restore homeostasis can be impaired by repetitive epithelial injury due to persistent insults such as chronic inflammation. The dysregulated process of wound healing can lead to abnormal remodeling of the sub-epithelial tissue characterized by the activation of fibroblasts and excessive deposition of ECM. One of the goals was to construct an organoid-based advanced in vitro model capable of emulating these salient features of fibrotic tissue remodeling in the intestine.

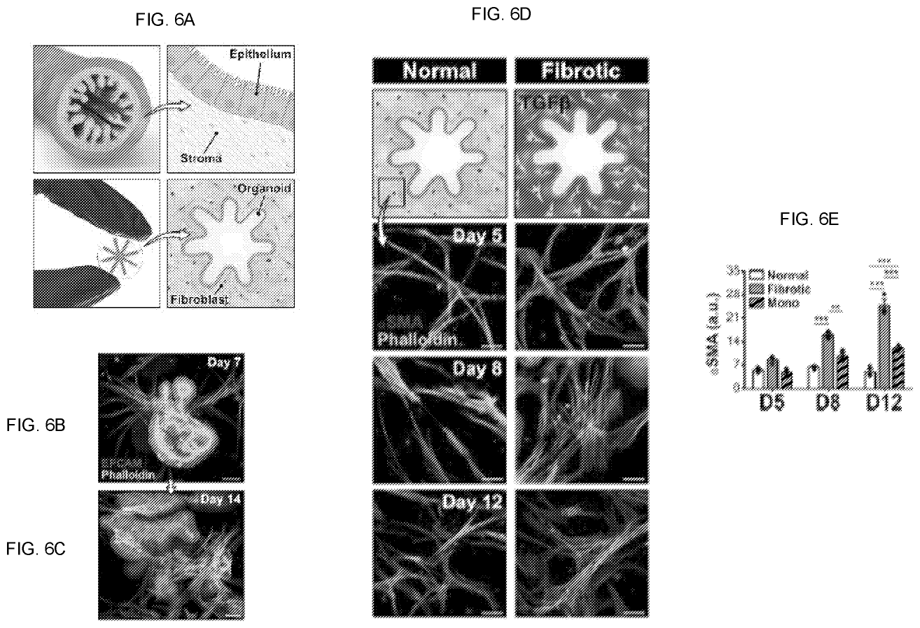

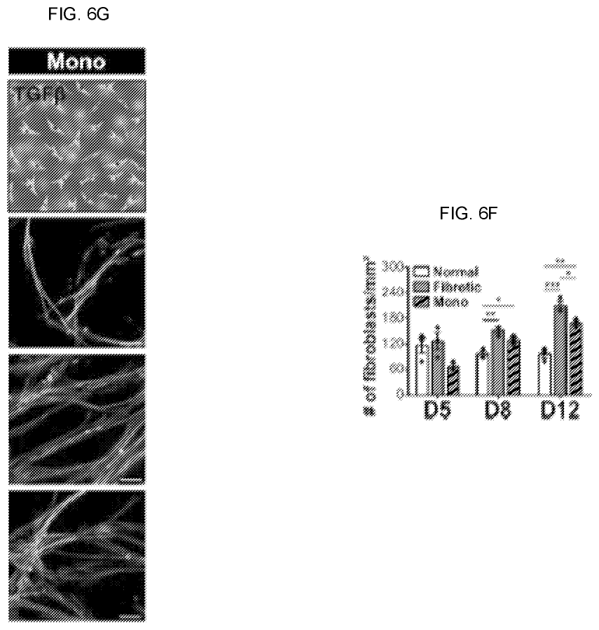

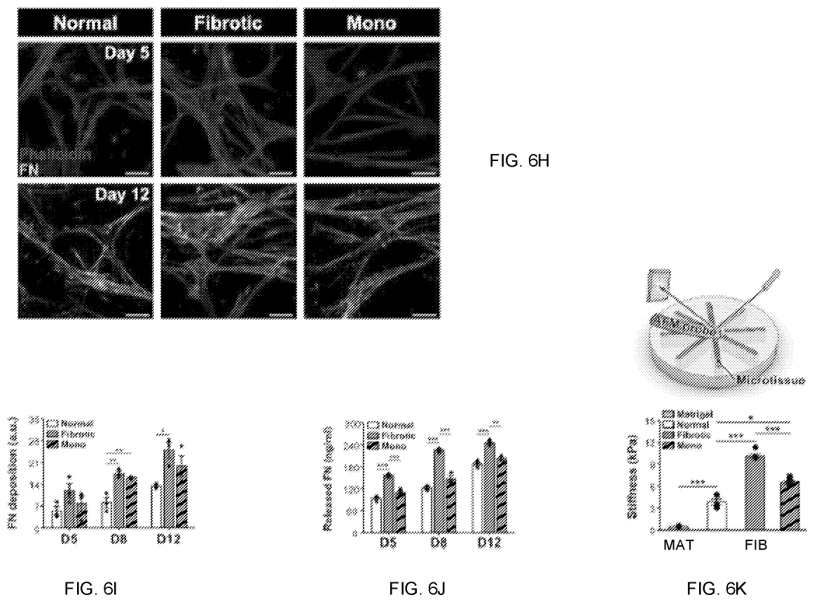

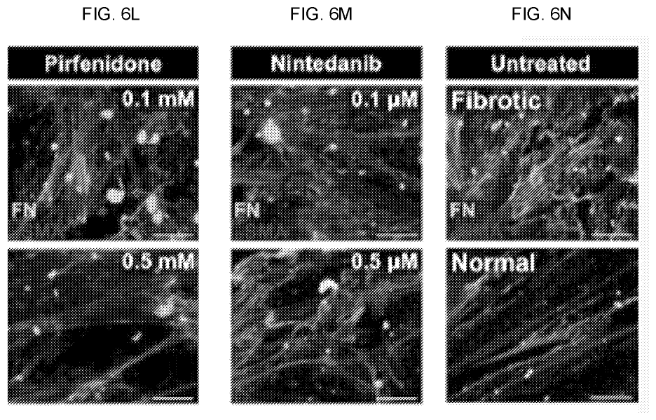

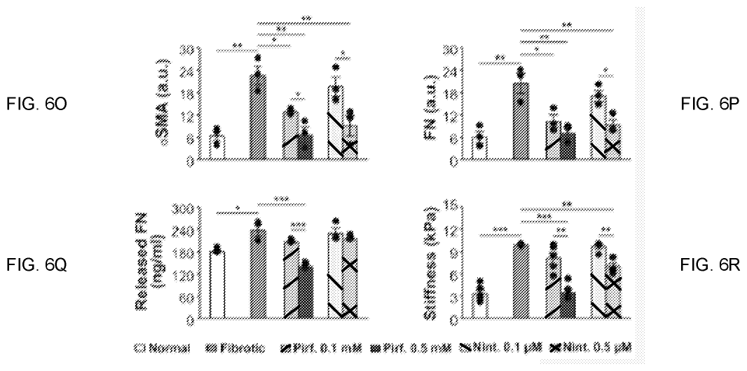

To this end, OCTOPUS was used to set up a co-culture of intestinal organoids and primary intestinal fibroblasts in the same hydrogel scaffold and generate a multicellular construct reminiscent of the intestinal epithelium and its underlying stroma in vivo (FIG. 6A). In this mixed co-culture configuration, the intestinal progenitor cells embedded in Matrigel developed into or-ganoids over the course of 5 days or so, during which fibroblasts began to spread and proliferate around the nascent organoids. Despite their active proliferation, the fibroblasts did not appear to impede organoid growth, nor did they cause any significant changes in the morphological characteristics of organoids (FIG. 6B). Prolonged culture in this device led to the formation of microtissues densely populated with enlarged organoids and fibroblasts (FIG. 6C).

To simulate the scenario of using this system to assess intestinal fibrosis in a conventional laboratory setting, a common technique widely used for in vitro modeling of fibrosis, which was to treat the co-culture construct with transforming growth factor (TGF)-β, were used. TGF-β plays a central role in the pathogenesis of fibrosis in the intestine and other organs by inducing the activation of fibroblasts and their transdifferentiation into myofibroblasts, which are the key effector cells that drive fibrogenesis. To trigger fibrogenic responses, the model was treated with TGF-β at 1 ng/ml from day 5 to day 12. Exposure of the intestinal microtissue to this condition indeed caused the fibroblasts to acquire the contractile phenotype of myofibroblasts, as evidenced by robust expression of alpha-smooth muscle actin (αSMA) (FIG. 6D). In comparison to untreated tissues, the level of expression increased by more than 1.9- and 2.9-fold by day 8 and day 12, respectively (FIG. 6E). TGF-β treatment also promoted fibroblast proliferation, yielding approximately twice as many cells by day 12 compared to the untreated control group (FIG. 6F). Notably, these TGF-β-induced changes in fibroblasts were reduced considerably when the intestinal organoids were removed from the model (FIG. 6E to FIG. 6G), suggesting significant epithelial contributions to the fibrogenic responses.

The model also permitted the investigation of ECM deposition, which is essential to fibrotic tissue remodeling. This analysis focused on fibronectin (FN) as a representative ECM protein.

During the same treatment period (from day 5 to day 12), immunostaining showed that stimulation with TGF-β substantially increased FN in the pericellular regions of fibroblasts as compared to the untreated tissues (FIG. 6H). The difference between the two groups became evident within 3 days of treatment and remained statistically significant throughout the culture period (FIG. 6I). This increased deposition of FN demonstrated by immunofluorescence was verified by ELISA of conditioned media that revealed higher levels of released FN in the intestinal tissues treated with TGF-β (FIG. 6J). Consistent with the analysis of fibroblast activation and proliferation (FIG. 6E, FIG. 6F), from the comparison between co-culture and monoculture models, intestinal organoids potentiate the fibrogenic effect of TGF-β and promote FN production by fibroblasts (FIG. 6I, FIG. 6J) were identified.

To further validate the fibrotic phenotype of the model, the stiffness of the TGF-β-treated microtissues was measured by using atomic force microscopy (AFM). This measurement was greatly facilitated by the open-top design of OCTOPUS that allowed direct access of the AFM probe to the tissue constructs in the culture chambers (FIG. 6K). When a blank Matrigel scaffold formed in OCTOPUS was tested, the stiffness was approximately 440 Pa but the co-culture of intestinal organoids and fibroblasts in the same type of matrix for 14 days increased the measured value to 3.5 kPa, which was comparable to the stiffness of healthy intestinal tissues in vivo (FIG. 6K). As expected, treatment of this model with TGF-01 led to a marked increase in the extent of tissue stiffening and generated co-culture constructs that were 2.9 times stiffer than those in the untreated devices (FIG. 6K). Interestingly, the average tissue stiffness in this case (10.1 kPa) was within the physiological range of fibrotic intestinal tissues measured in vivo (FIG. 6K). Taken together, these results demonstrate the feasibility of engineering the microenvironment of organoids in OCTOPUS to mimic the progressive process of matrix remodeling and stiffening during the development of intestinal fibrosis.

The utility of the intestinal fibrosis model for drug testing applications was tested. Given that no specific therapies are currently available for intestinal fibrosis, two anti-fibrotic drugs, Pirfenidone and Nintedanib, approved for the treatment of idiopathic pulmonary fibrosis (IPF), were used. Although these drugs were developed for fibrosis in the lung, they can modulate the activity of fibrogenic pathways in other organs such as the heart, kidney, liver, and skin. These findings led to an examination of whether the com-pounds would have similar therapeutic effects on intestinal fibrosis. The potential of the drugs to reverse the fibrotic phenotype of the model, namely fibroblast activation and excessive ECM deposition, was assessed.

First, fibrotic intestinal tissue constructs in OCTOPUS were generated by forming co-culture organoids over 5 days and exposing them to TGF-β for another 7 days as described above (day 5-day 12). These constructs were then treated with clinically relevant concentrations of the drugs for 48 hours (day 13-day 14) within the therapeutic window identified by viability assessment (data not shown). In a control group, the fibrotic tissues did not receive drug treatment during the 48-hour period. At 0.1 mM, Pirfenidone was effective for altering the contractile phenotype of fibroblasts, as illustrated by 50% reduction in αSMA compared to the untreated control (FIGS. 6L to 6O). The expression of αSMA was further decreased when the dose was increased to 0.5 mM (FIGS. 6L to 6O). The effect of Pirfenidone on ECM remodeling was evident from significantly decreased immunofluorescence of FN in the drug-treated fibrotic constructs (FIGS. 6L to 6P). Consistent with this result, ELISA analysis indicated that Pirfenidone also reduced the amount of FN released from the model (FIG. 6Q). Important to note is that the levels of αSMA expression and FN production after higher-dose (0.5 mM) treatment were statistically indistinguishable from those measured in the normal group representing healthy intestinal tissue (FIGS. 6O to 6Q), demonstrating the capacity of Pirfenidone to reverse TGF-β-induced fibrosis in the model and rescue the normal phenotype.

In the case of Nintedanib, a lower dose of the drug (0.1 μM) failed to exert significant effects on the fibrosis model (FIGS. 6M to 6Q). When the drug concentration was raised to 0.5 μM, however, both fibroblast activation and FN accumulation decreased substantially to the levels comparable to those achieved by Pirfenidone treatment at 0.5 mM (FIGS. 6L to 6Q). In this case, the concomitant reduction in the concentration of released FN (FIG. 6Q) was not observed, suggesting that αSMA and FN deposition within the tissue are better indicators of the therapeutic efficacy of Nintedanib.

Consistent with the results of immunofluorescence analysis, AFM data demonstrated the anti-fibrotic effects of Pirfenidone and Nintedanib in a dose-dependent manner (FIG. 6R). While both drugs significantly decreased the stiffness of the TGF-β-treated fibrotic tissues when administered at higher doses, Pirfenidone appeared to have more pronounced effects as evidenced by the greater extent of tissue softening (FIG. 6R). The average stiffness measured in the fibrosis model treated with 0.5 mM Pirfenidone (3.5 kPa) closely matched that of normal tissue constructs (3.3 kPa), illustrating the potential of Pirfenidone to normalize the mechanical property of fibrotic intestinal tissues in the model system.

In response to the increasing need for new technologies for organoid research, here a microengineered platform was established to reconfigure the three-dimensionality of conventional organoid culture. OCTOPUS introduced in this paper provides a simple yet effective means to address the problem of limited nutrient supply inherent in 3D culture and engineer a more uniform, unrestricted soluble environment beneficial for long-term culture of organoids. As demonstrated by the model systems, the extended lifespan of organoids significantly increased their size and maturity beyond what is achievable using conventional techniques and enabled the production of more realistic multicellular constructs for in vitro modeling of organogenesis and disease development.

Organoids in conventional hydrogel drop scaffolds can be passaged weekly for prolonged periods to increase their in vitro lifespan. Mechanically disrupted organoids during subculture have the capacity to rapidly seal themselves and restore their original architecture and functional properties. As demonstrated by the long-term culture of intestinal organoids for over 1 year, this approach has proven instrumental for expanding organoids and maintaining their differentiated phenotype over extended periods. The increased lifespan of organoids in this case, however, does not necessarily translate into enhanced tissue maturity because frequent passaging (typically every 5-7 days) required by conventional culture protocols disrupts the process of sustained organoid development and maturation. OCTOPUS resolves this issue by enabling uninterrupted, continuous culture of organoids for significantly longer (>3×) periods of time.

Although the ability to support continuous long-term culture is the key advantage of OCTOPUS, the data also revealed other desirable features of organoid development in this system. After 7 days of culture, for example, virtually every marker of structural and functional maturation measured was expressed in significantly higher levels in OCTOPUS-generated or-ganoids (FIG. 3). These results suggest that OCTOPUS is also capable of accelerating the growth and maturation of organoids at the early stage of development. Presumably, this can be explained by more rapid and uniform diffusion of soluble signals in the device that allows OCTOPUS to more effectively keep up with the rapidly increasing metabolic needs of nascent organoids.

By leveraging these capabilities, the work also demonstrated the feasibility of developing a specialized organoid model in OCTOPUS that can simulate the salient features of dysregulated fibrogenesis during the development of intestinal fibrosis. The disclosed subject matter entailed a sequential process of generating co-culture organoid constructs and then exposing them to a fibrogenic factor, which took place over time periods (12-14 days) well beyond the typical lifespan of organoids in conventional culture. Indeed, a rapid loss of organoid viability in Matrigel drops after day 5 made it challenging, if not impossible, to model fibrogenic responses to TGF-β (data not shown), which began to occur only after 8 days of continuous culture in OCTOPUS and became more evident over time (FIG. 6E, FIG. 6F). Despite its simplicity, the fibrosis model approximated the extent of tissue stiffening measured in vivo and revealed the significant role of the intestinal epithelium in fibrotic tissue remodeling. Furthermore, the proof-of-principle for using this disease model was shown as a drug testing platform. The anti-fibrotic effects of Pirfenidone and Nintedanib used have already been established in other organs, but the results provide in vitro evidence that supports the possibility of extending their use to intestinal fibrosis. Also, it is important to highlight the potential of the model for applications in high-content drug screening as illustrated by the use of various analytical techniques for in situ measurement of drug responses, including microfluorimetry, ELISA, and AFM. Considering that the pathophysiological processes underlying the development of fibrosis are conserved across organs, the same device and in vitro techniques can be applicable to modeling fibrotic diseases and their pharmacological modulation in other organs.

While OCTOPUS represents considerable changes to the design of traditional organoid models, the implementation of this system does not require any modification of established culture protocols and workflow, nor does it rely on specialized equipment or personnel. Essential to this advantage is the design of OCTOPUS as a ready-to-use and easily-accessible culture insert that is directly compatible with standard well plates and laboratory infrastructure. As exemplified by the intestinal models, generating mature organoids in OCTOPUS can readily be accomplished in traditional laboratory settings based on materials and experimental procedures commonly used in conventional techniques. This is an important aspect of the method that makes OCTOPUS an immediately deployable and readily accessible culture platform, which can contribute to the rapid dissemination of the technology for widespread use.

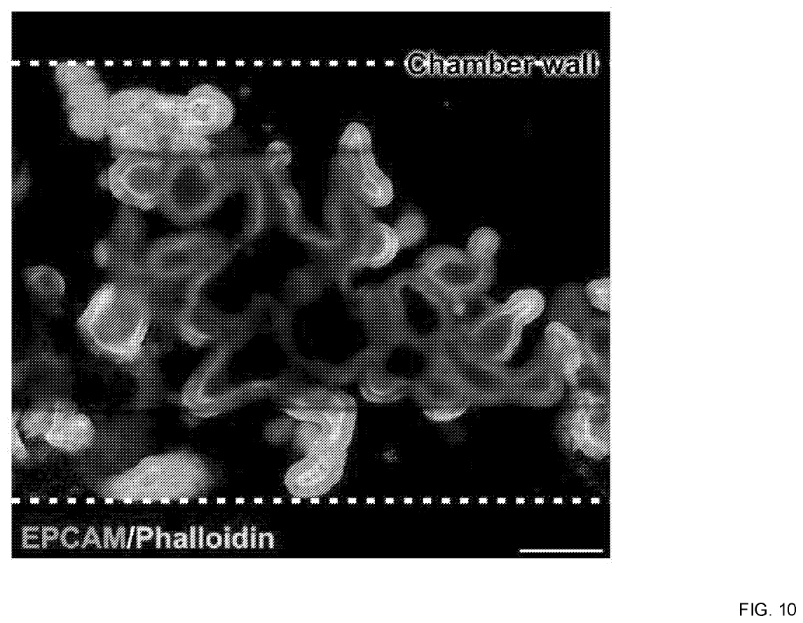

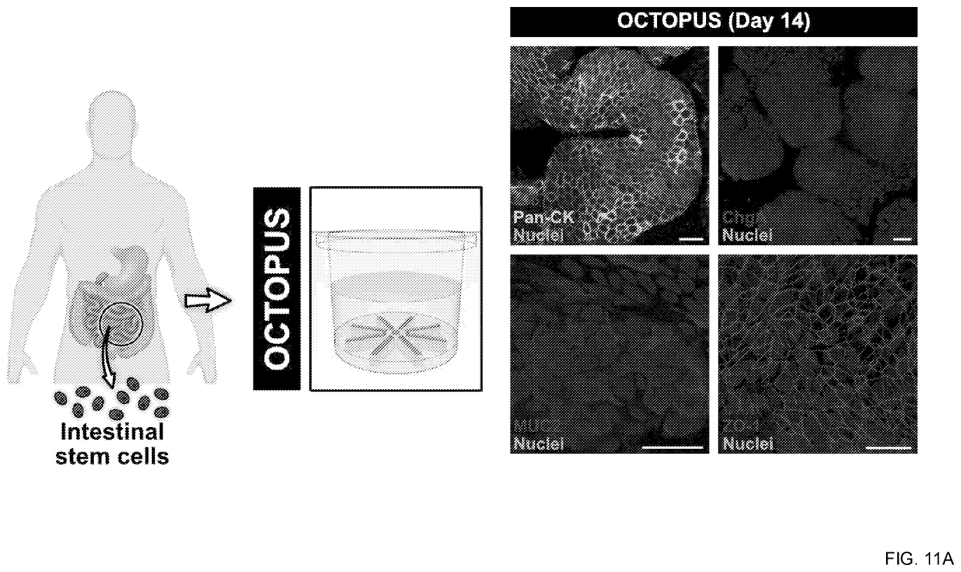

The foregoing demonstration raises several fundamental questions that open new avenues for further investigation. Among them is which design parameters play a significant role in the long-term development of organoids in OCTOPUS. During extended culture, intestinal organoids in the device continued to grow in size as shown in FIG. 2F and eventually touched the surface of the culture chambers (FIG. 10), after which their further enlargement in the lateral direction was physically restricted by the walls. This observation suggests that the size of the culture chamber is an important consideration for the sustained growth of organoids in OCTOPUS. Given that the chamber geometry is readily adjustable during device fabrication, the patterns of organoid development in enlarged culture arrays can be assessed, and the size and shape of the chambers can be optimized, with the goal of engineering a 3D culture environment that remains unrestricted both physically and biochemically.

Perhaps the more important question is how long the system can support continuous growth and maturation of organoids before passaging becomes necessary. Many intestinal organoids in the device ceased to grow after 4 weeks of culture (data not are shown), which can be considered conservatively as the maximum duration of continuous culture of small intestinal organoids in the current design of OCTOPUS. This result begs the question of whether the arrested growth of organoids is due to the physical constraints of the culture chambers described above. Another explanation is that the dead cells sloughing off of the epithelium during the natural process of epithelial turnover and accumulating in the closed liminal cavity can exert deleterious effects on the organoids, which has been described previously in the same type of organoids. It can be that as organoids grow beyond a certain limit, the system in its current configuration reaches its maximum capacity and becomes no longer capable of meeting the metabolic demands of enlarged organoids. As discussed above, analysis of organoid development in culture chambers with different sizes and geometry can help address some of these questions. The external environment of OCTOPUS can be modified to facilitate diffusion in organoid culture scaffolds. For example, an orbital shaker can be used to agitate media and generate convective flow in OCTOPUS-containing culture wells as a simple strategy to increase the rate of diffusion, which can contribute to further improving the longevity of organoid models.

Finally, emulating the maturity of native organs in organoid models will require advanced approaches beyond the enhancement of nutrient supply and cell viability demonstrated here to account for the integrated biological complexity of in vivo systems. The assessment is based on the rationale that the short lifespan of organoids is the primary reason for their limited ability to reach later stages of development and acquire mature phenotype. From a developmental biology perspective, however, there is increasing recognition that the limited maturity of organoids in conventional models can also be due to the absence of the surrounding embryonic tissues of developing organs in vivo that provide instructive cues to guide the process of organ development and maturation. Recapitulating this critical aspect of organogenesis in vivo can greatly enhance the ability of OCTOPUS to promote structural and functional maturation of organoids. The results from the co-culture organoid models (FIG. 5) indeed demonstrate the feasibility and potential benefit of this approach—the intestinal organoids grown with their associated fibroblasts showed significantly enhanced growth and maturation in comparison to the monoculture control (FIG. 5F, FIG. 5G). Although preliminary, these findings warrant further investigation of whether mimicking the collection of specialized tissues in native organs and their biological interactions can be leveraged as a complementary strategy for developing more mature and realistic organoid constructs in OCTOPUS.

Developing new in vitro technologies for laboratory production and maintenance of organoids is emerging as a major area of investigation in organoid research. Representing this new trend, the work provides a good example of how rational design engineering of conventional organoid culture can advance the ability of organoids to emulate the structural and functional complexity of their in vivo counterparts. By seamlessly integrating engineering novelty into traditional in vitro techniques, the technology offers a simple, practical 3D culture strategy that can be implemented immediately to expand the capabilities of current organoid models. OCTOPUS has the potential for a significant impact on organoid technology and can also provide a powerful platform for various other applications that involve cell and tissue culture in 3D environments.

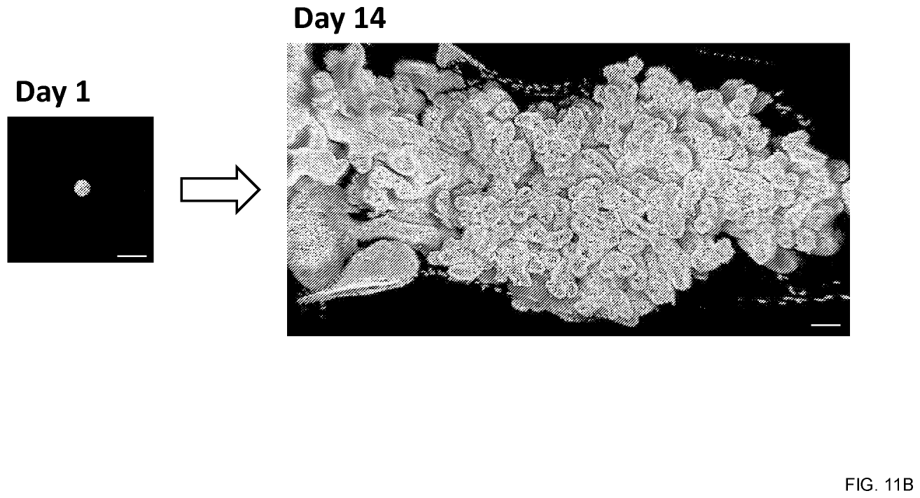

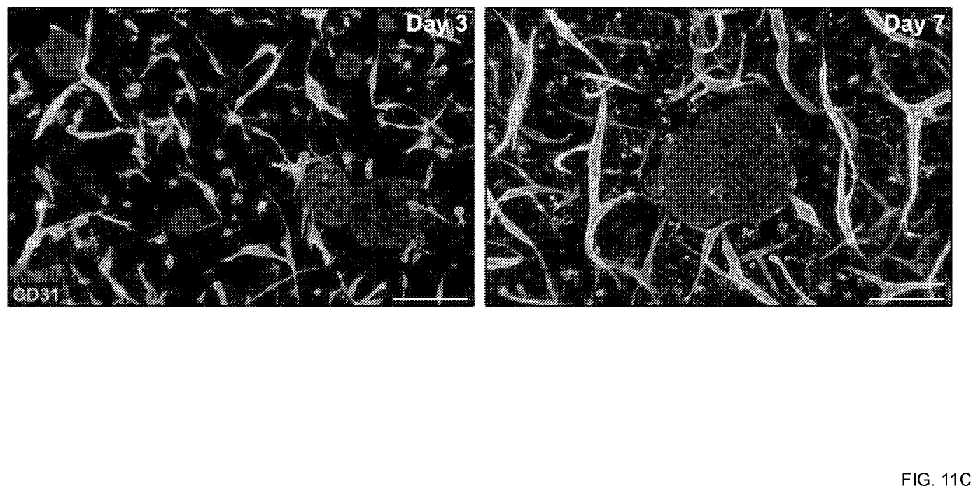

FIG. 11A to FIG. 11C shows that human organoids can be cultured in OCTOPUS for a long-term period. For example, human intestinal organoids can be cultured in OCTOPUS for more than 14 days (FIG. 11A) to provide larger and more differentiated tissue phenotype compared to day 1 organoids (FIG. 11B). The human organoids can be vascularized by co-culturing them with endothelial cells in a 3D microenvironment (FIG. 11C).

Investigations into human organoid models will be described in greater detail below.

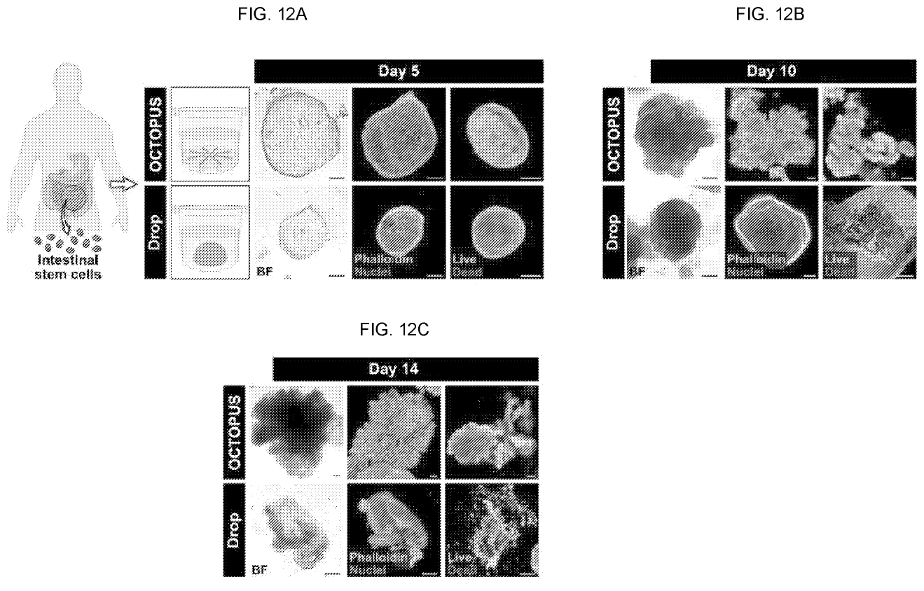

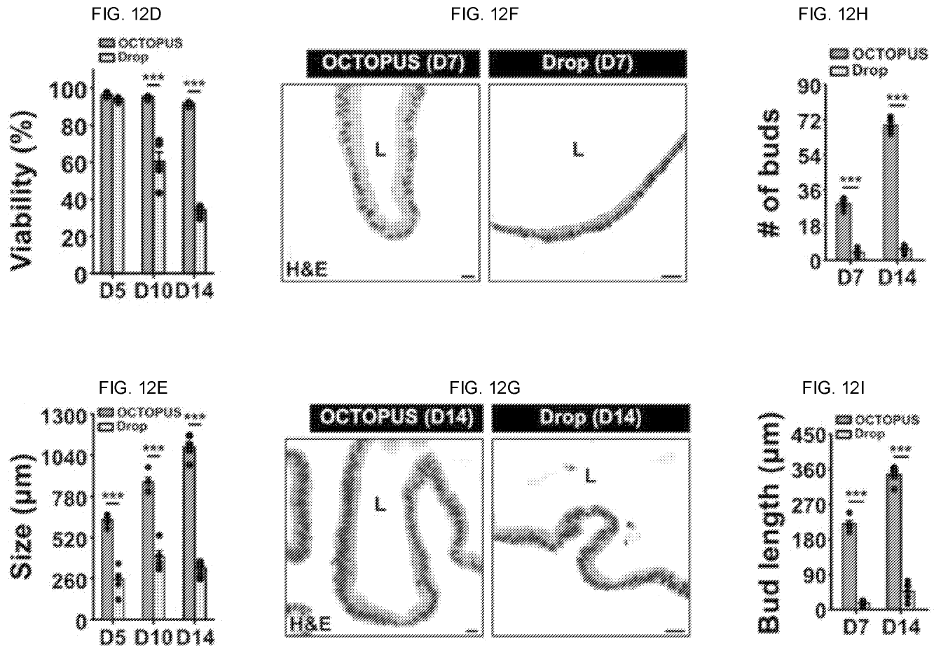

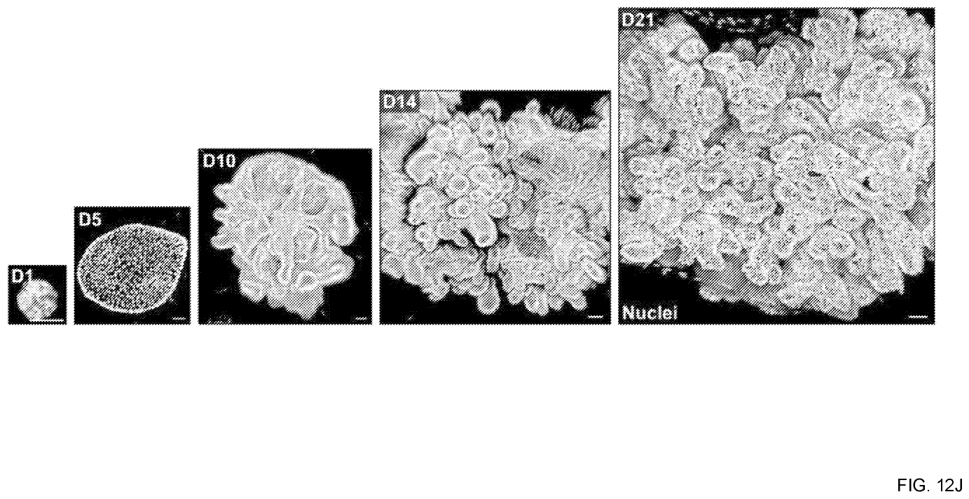

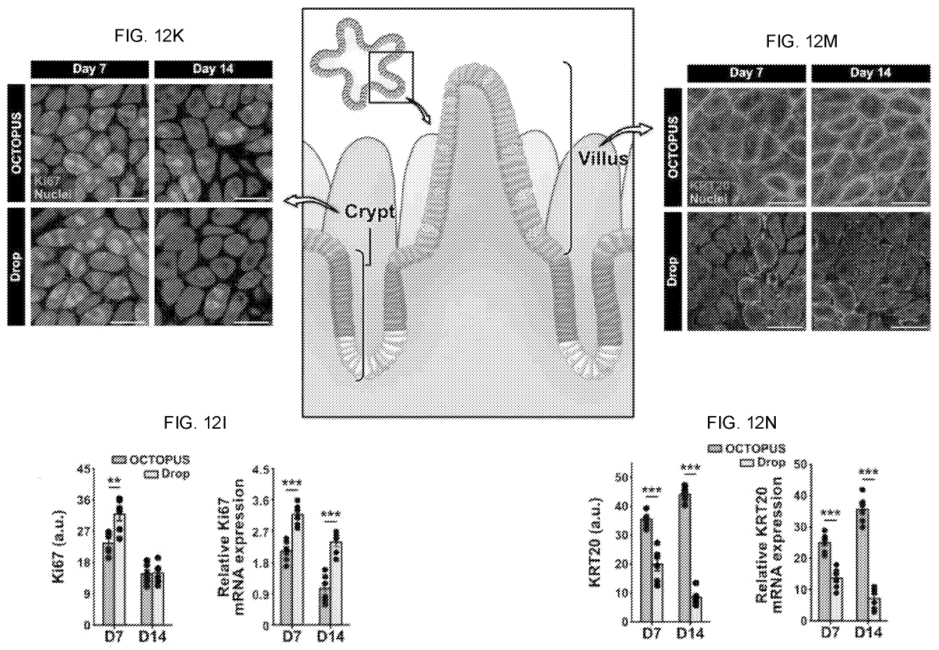

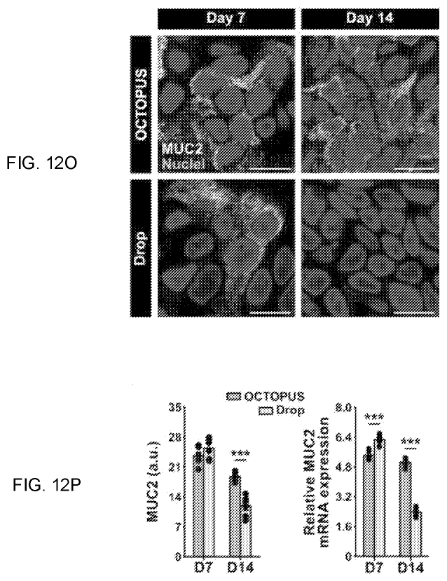

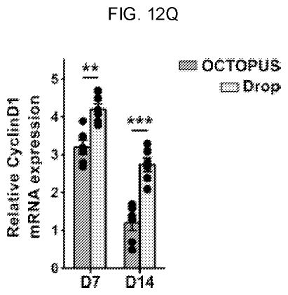

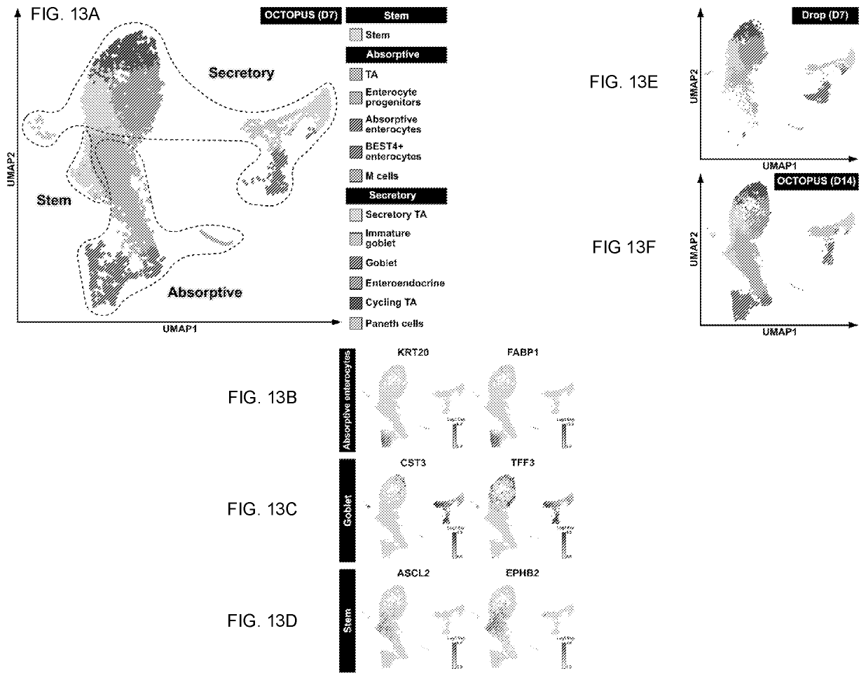

Having demonstrated the proof-of-concept of OCTOPUS using mouse organoids, the applicability of this technology to human intestinal organoids was explored. To this end, single cell suspension isolated from the small intestine (terminal ileum) of healthy donors were cultured and their self-organization and epithelial differentiation in the disclosed device and Matrigel drops (FIG. 12A) was examined. During the first 5 days of culture, the formation of spherical organoids with high cell viability was observed in both methods (FIG. 12A, FIG. 12D), but the ones in OCTOPUS were found to be significantly larger (FIG. 12A, FIG. 12E). As culture progressed, the cystic organoids in the disclosed device continued to grow without measurable cell death and underwent morphological transformation into budding structures that extended epithelial folds into the surrounding matrix (FIG. 12B to FIG. 12D). This developmental process was not observed in Matrigel drops in which prolonged culture over 14 days resulted in arrested growth and more than 50% reduction in cell viability without evidence of bud formation (FIG. 12B to FIG. 12E). These microscopic findings were supported by histological analysis that showed the presence of bud-like structures in the OCTOPUS enteroids at day 7 (FIG. 12F), which continued to increase in both number and length during extended culture (FIG. 12G to FIG. 12I). In contrast, intestinal organoids in Matrigel drops developed considerably fewer and smaller buds (FIG. 12F to FIG. 12I) and remained mostly spherical throughout the culture period. Importantly, continuous, uninterrupted culture in OCTOPUS over 1 month without passaging led to a more than 32-fold increase in size, yielding human enteroids with extensive epithelial folding that were as large as 2.6 mm in diameter (FIG. 12J).

Closer examination of the cultured constructs revealed the presence of distinct cell populations and their localized spatial distribution within the developing organoids. For example, proliferative cells identified by positive Ki67 immunostaining were found predominantly at the tip of the buds corresponding to the crypt region (FIG. 12K). This observation was verified by RT-PCR analysis of genes encoding Ki67 (FIG. 12I) and CyclinD1 (FIG. 12Q), a protein that mediates the G1/S transition in the cell cycle. The organoids also contained cells that expressed a marker of differentiated absorptive enterocytes (KRT20) (FIG. 12M). Important to note is that KRT20 expression in OCTOPUS culture was significantly higher than that in Matrigel drops, as illustrated by both immunofluorescence and mRNA expression (FIG. 12M, FIG. 12N). Analysis of the disclosed device also revealed increasing induction of KRT20 extending from day 7 to day 14 (FIG. 12M, FIG. 12N), presumably due to sustained epithelial development and maturation during prolonged culture. This increase was accompanied by a decrease in the expression of Ki67 and CyclinD1 over the same period (FIG. 12L, FIG. 12Q), suggesting reduced cell proliferation due to increased differentiation of the intestinal epithelium. This inverse relationship was not observed in Matrigel drops, in which case both cell differentiation and proliferation decreased over time (FIG. 12L, FIG. 12N, FIG. 12Q).

The enhanced epithelial maturation of enteroids in OCTOPUS was further evidenced by similar trends in the induction of a goblet cell-specific marker (MUC2) (FIG. 12O, FIG. 12P). Taken together, these results demonstrate the feasibility of using OCTOPUS to generate human intestinal organoids and support their sustained development to achieve size and tissue maturity not attainable in conventional Matrigel drop culture.

Recognizing the intrinsic capacity of human intestinal stem cells to give rise to various cell types during organoid development, scRNA-seq analysis was performed to investigate cellular heterogeneity of human enteroids in OCTOPUS. For this study, enteroids were harvested from the disclosed devices at days 7 and 14, and their single-cell transcriptional profiles were examined in comparison to those cultured in Matrigel drops for 7 days—sequencing data from 14-day Matrigel drop culture were excluded in the analysis to avoid confounding factors due to significant cell death observed in this group (FIG. 12D).

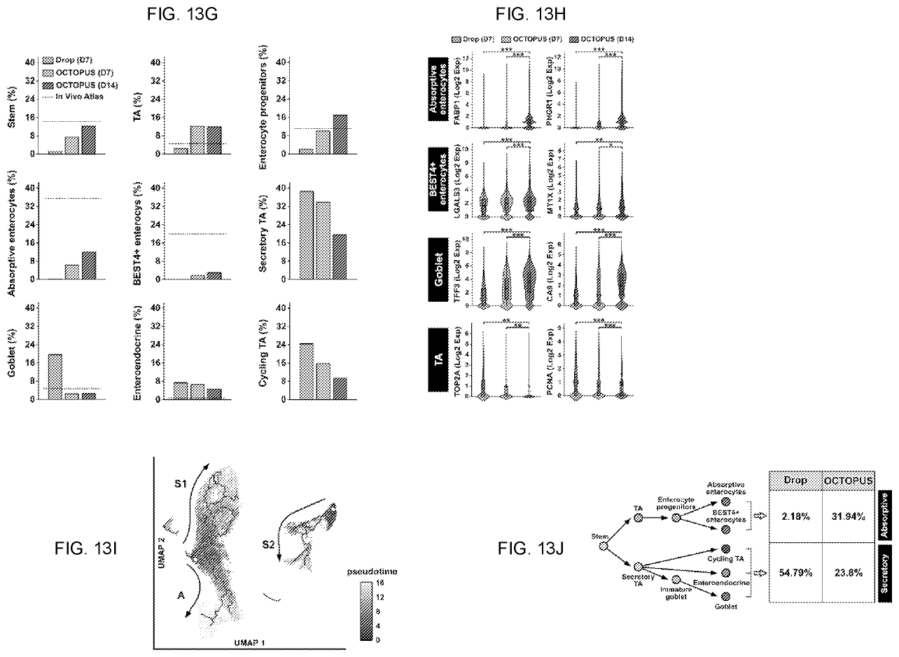

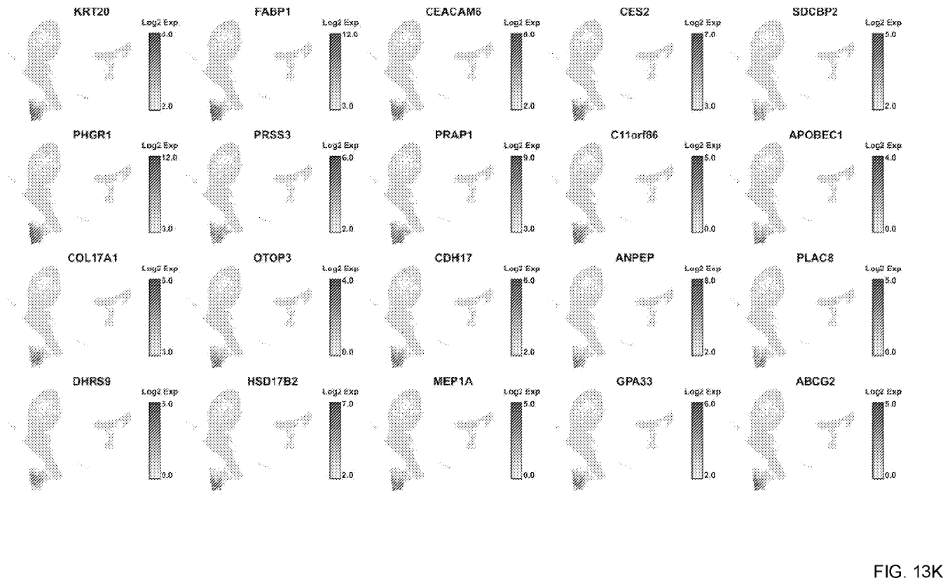

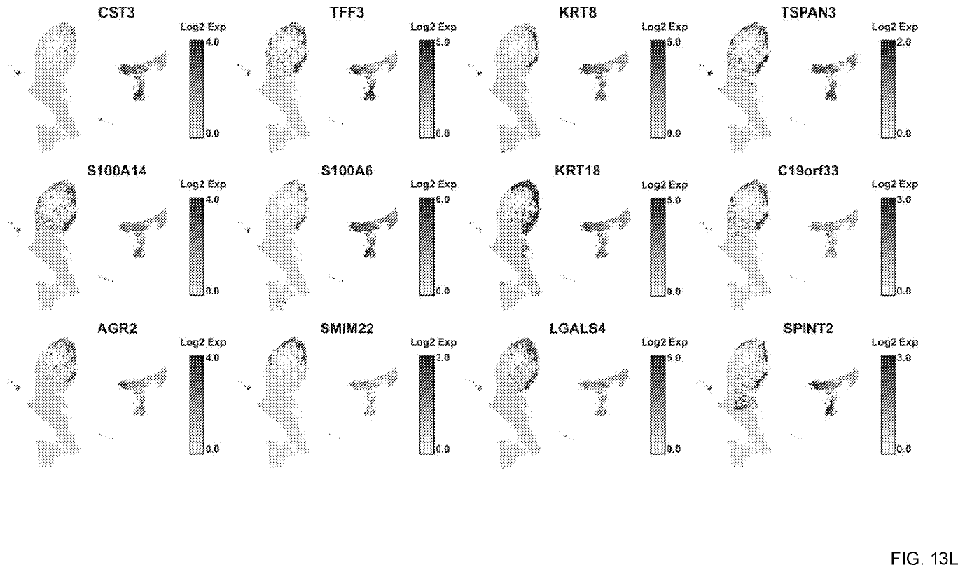

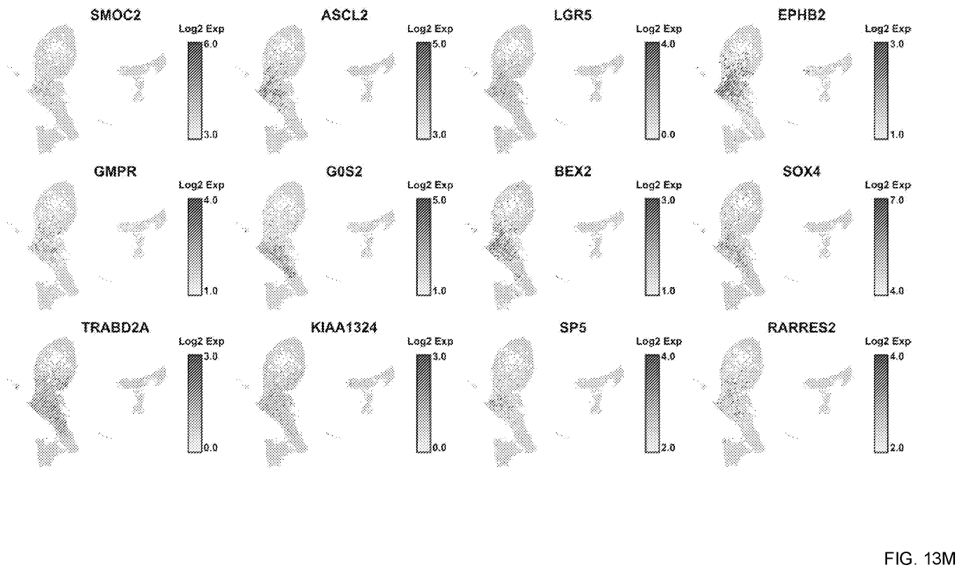

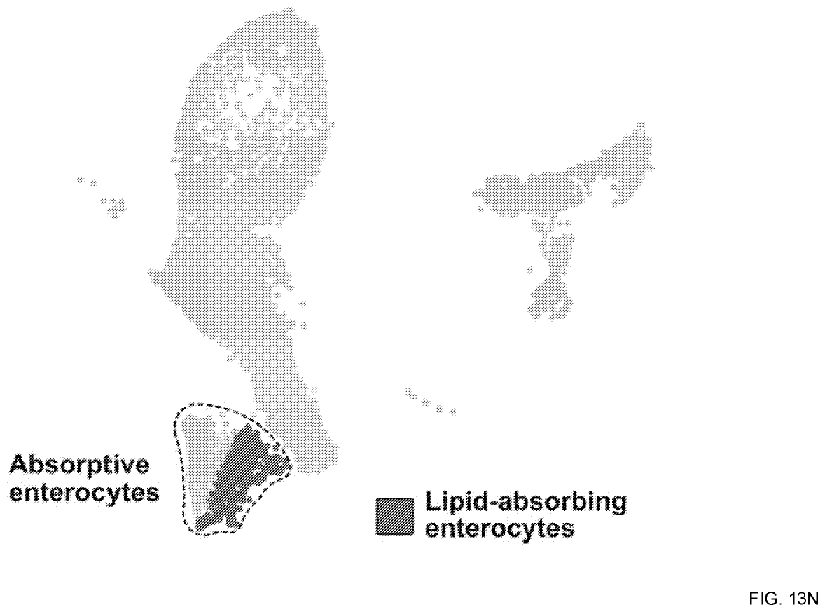

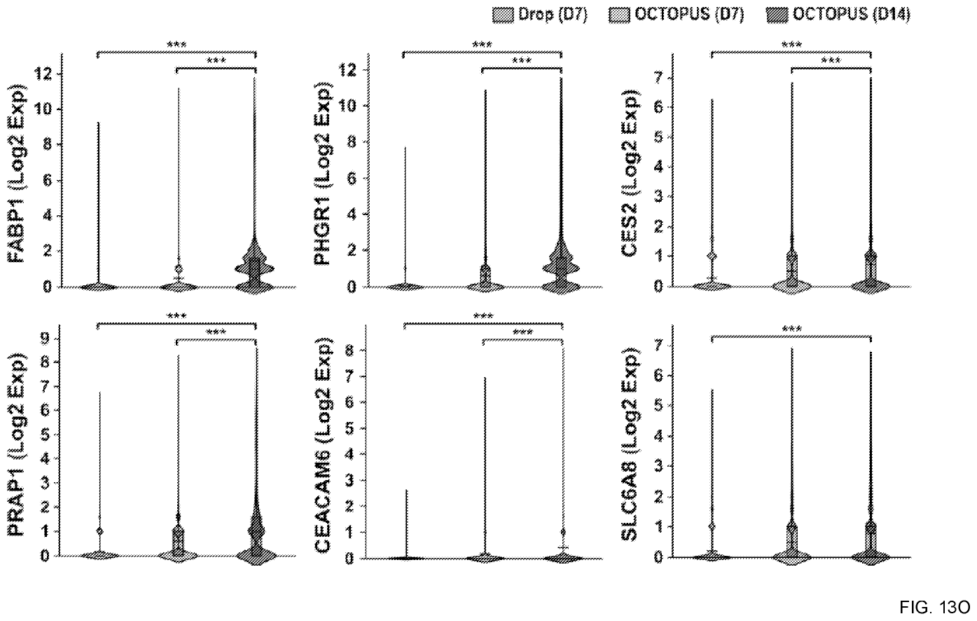

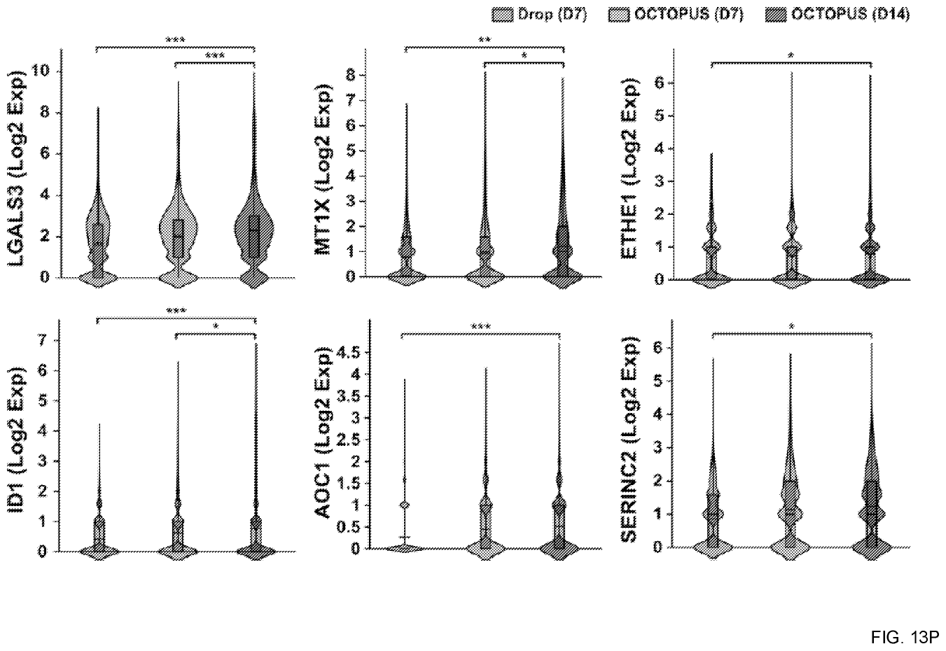

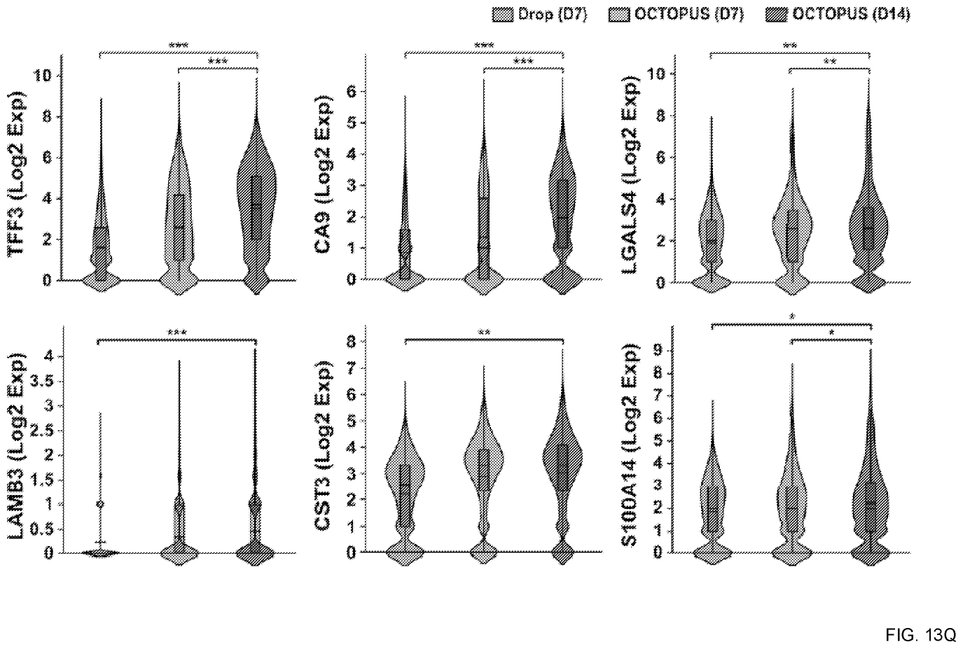

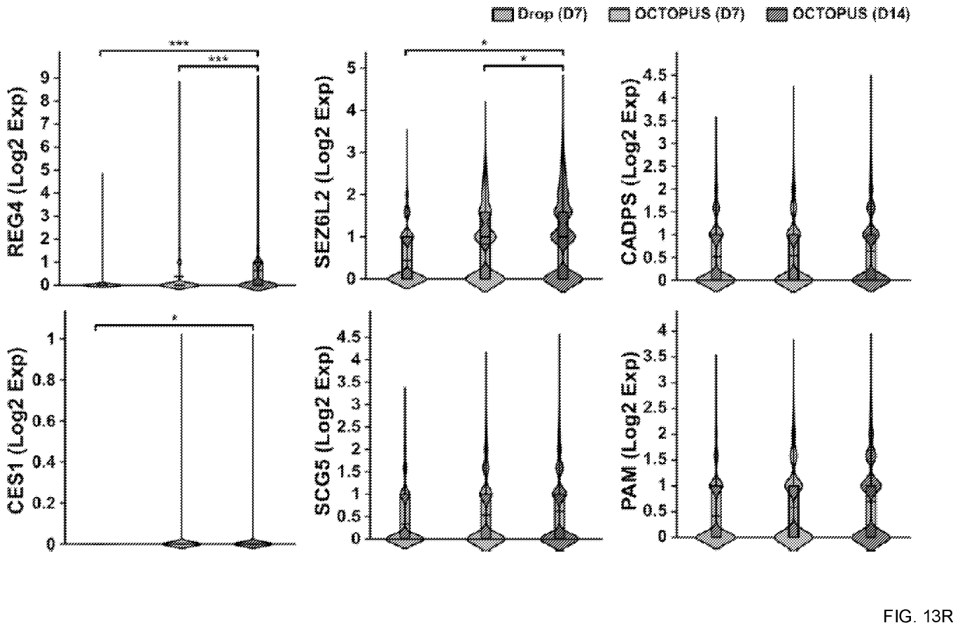

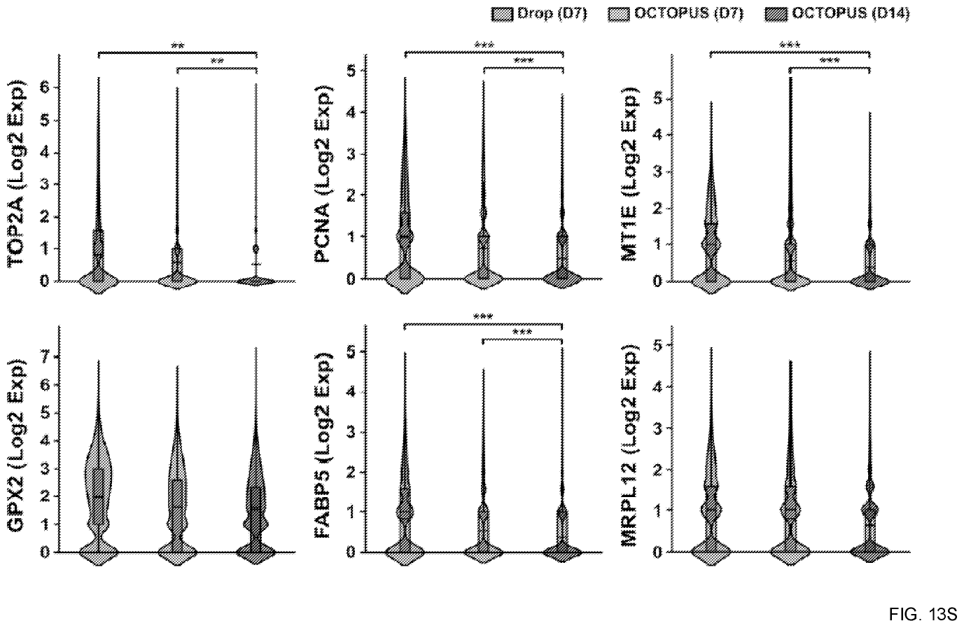

Uniform manifold approximation and projection (UMAP) clustering of the sequencing data obtained from OCTOPUS at day 7 yielded 3 broadly defined groups of cells—absorptive cells, secretory cells, and stem cells—each of which contained multiple subpopulations distinctly identified by the expression of cell-type-specific genes described in previous in vivo studies of the human small intestine (FIG. 13A). Specifically, the absorptive cell group was composed of 5 transcriptionally distinct cell types, including absorptive enterocytes, enterocyte progenitors, bestrophin-4 (BEST4)-positive enterocytes, absorptive transit-amplifying (TA) cells, and M cells (FIG. 13A). The absorptive enterocyte cluster in this group, for example, was defined by high expression of enterocyte-specific transcripts known to regulate absorptive function of the intestinal epithelium, such as Keratin 20 (KRT20), Fatty acid binding protein 1 (FABP1), and Carcinoembryonic antigen-related cell adhesion molecule 6 (CEACAM6) (FIG. 13B, FIG. 13K). The secretory cell group contained 6 clusters (FIG. 13A), one of which represented goblet cells identified by the expression of cystatin C (CST3), trefoil factor 3 (TFF3), and S100 calcium binding protein A14 (S100A14) (FIG. 13C, FIG. 13L). Clustering of stem cells was based on the expression of intestinal stem cell markers, including achaete-scute complex homolog 2 (ASCL2), ephrin type-B receptor 2 (EPHB2), and SPARC related modular calcium binding 2 (SMOC2) (FIG. 13D, FIG. 13M).

Importantly, the sequencing results revealed cell populations uniquely present in OCTOPUS. A good example of such cell types is a subset of the absorptive cell population expressing BEST4 (BEST4+ enterocytes) (FIG. 13A), which plays a critical role in host-microbiome interactions and various homeostatic functions in the intestine such as ion transport. This specialized cell type was not found in human enteroids generated by Matrigel drop culture (FIG. 13E). Lipid-absorbing enterocytes identified by their expression of apolipoproteins (APOA4, APOC3, APOA1, ALPI, and APOB) are another subpopulation of absorptive enterocytes produced by the disclosed device culture (FIG. 13N) that was absent in Matrigel drops (FIG. 13E). Comparison of cell clusters in these two systems also showed substantially higher abundance of absorptive cell lineages in OCTOPUS (FIG. 13A, FIG. 13E). When the duration of culture was extended from 7 to 14 days in the OCTOPUS group, spatial distribution of the identified cell populations remained largely unchanged (FIG. 13F). Many of the clusters, however, were seen with noticeable changes in their density, indicating altered cellular abundance during prolonged culture.

To further examine these changes and understand their relevance to the native system, the cellular makeup of the enteroids in OCTOPUS in comparison to the previously published single-cell atlas of the human intestinal epithelium in vivo was analyzed. Results of this analysis showed several differences in epithelial composition between the culture conditions tested in herein. First, extended culture in OCTOPUS led to enrichment of stem cells far beyond what was achievable in Matrigel drop culture as evidenced by their increased proportion in the total population from 7.4% at day 7 to 12.3% by day 14, closely approximating the fraction of intestinal stem cells in vivo (14%) (FIG. 13G). Second, OCTOPUS permitted expansion of enterocyte lineages (absorptive enterocytes, BEST4+ enterocytes, enterocyte progenitors) in the absorptive cell population from day 7 to day 14 (FIG. 13G), which was in contrast to the small or negligible fraction of these cells in Matrigel drops. As a result, enterocyte progenitors reached and exceeded the physiological level of abundance after 14 days of culture. Despite expansion, however, the proportions of absorptive enterocytes and BEST4+ enterocytes were still significantly lower than those reported in the in vivo atlas. Third, the subpopulations of the secretory cell group displayed a general trend of decreasing abundance in OCTOPUS going from day 7 to day 14 (FIG. 13G). For the majority of cell types in this group, their proportions at day 14 were substantially smaller than those in Matrigel drops but the lower abundance served to better approximate the cellular composition of the secretory epithelium in vivo. For example, the fraction of goblet cells in OCTOPUS at day 14 (2.56%) was much more comparable to that in vivo (5%) than was measured in Matrigel drops (19.63%).

Sequencing data also revealed important time- and platform-dependent differences in transcriptional regulation of epithelial maturation. Among the key findings was significantly increased expression of genes specific to mature enterocytes as a result of prolonged culture in OCTOPUS. These genes included i) FABP1, PHGR1, PRAP1, and SLC6A8 for absorptive enterocytes (FIG. 13H, FIG. 13O) and ii) LGALS3 and MT1X expressed by BEST4+ enterocytes (FIG. 13H, FIG. 13P). Similar promotive effects of long term culture were observed in the maturation of the secretory cell populations in OCTOPUS at day 14, as illustrated by the expression of goblet cell-specific genes (TFF3, CA9, and S100A14) (FIG. 13H, FIG. 13Q) and enteroendocrine cell transcripts (REG4, SEZ6L2) (FIG. 13R).

Interestingly, in comparison to Matrigel drop culture, OCTOPUS enteroids showed significant downregulation of genes associated with the proliferative capacity of TA cells, such as TOP2A, PCNA, MT1E, and FABP5 (FIG. 13H, FIG. 13S). This result is consistent with previous in vivo reports that cell proliferation in the TA zone of the small intestine is suppressed with increasing tissue maturity during intestinal development, further supporting the capacity of OCTOPUS to enhance organoid maturation.

Finally, single-cell trajectory analysis was performed using Monocle to further characterize the dynamic process of stem cell differentiation during the development of human enteroids in OCTOPUS. When reconstructed in pseudotime on the UMAP plot, the developmental trajectory showed branching into two distinct domains representing absorptive and secretory cell lineages shortly after the initiation of culture (FIG. 13I). Following this initial lineage commitment, stem cells in the secretory cell domain proceeded to secretory TA cells, after which the differentiation trajectory branched into the cycling TA and enteroendocrine cell domains (arrow S1 in FIG. 13I). During the same period of pseudotime, separate clusters on the UMAP plot also showed the transition of secretory TA cells to immature goblet cells and then to goblet cells (arrow S2 in FIG. 13I). Tracing of the differentiation trajectory in the absorptive cell domain revealed developmental progression from stem cells to absorptive TA cells to enterocyte progenitors, which then gave rise to absorptive enterocytes and BEST4+ enterocytes (arrow A in FIG. 13I).

When combined with the proportion of each cell type, the developmental trajectories allowed for more quantitative characterization and direct comparison of stem cell differentiation in OCTOPUS and Matrigel drops (FIG. 13I, FIG. 13J). In conventional drop culture, data indicated that the development of stem cells was skewed heavily towards secretory cell lineages, accounting for more than 54.79% of the differentiated cells (FIG. 13J). By contrast, stem cell differentiation in OCTOPUS was directed more towards mature enterocytes to produce a significantly higher abundance of enterocyte progenitors, absorptive enterocytes, and BEST4+ enterocytes in the enteroids, which together constituted more than 31.94% of the epithelial population (FIG. 13J). Given the role of enterocytes as the most numerous cell type responsible for organ-specific function of the small intestine, this comparison provides evidence supporting the advantage of OCTOPUS for engineering human enteroids with more physiological cellular composition and enhanced functional capacity.

Recognizing that in vitro modeling of complex disease is emerging as an active area of investigation in organoid research, efforts shifted to demonstrating the proof-of-principle of using OCTOPUS to construct organoid-based disease models with increased fidelity and physiological relevance. Building upon work on human enteroids, focus moved to modeling human inflammatory bowel disease (IBD), which represents a group of diseases characterized by chronic inflammation of the gastrointestinal tract.

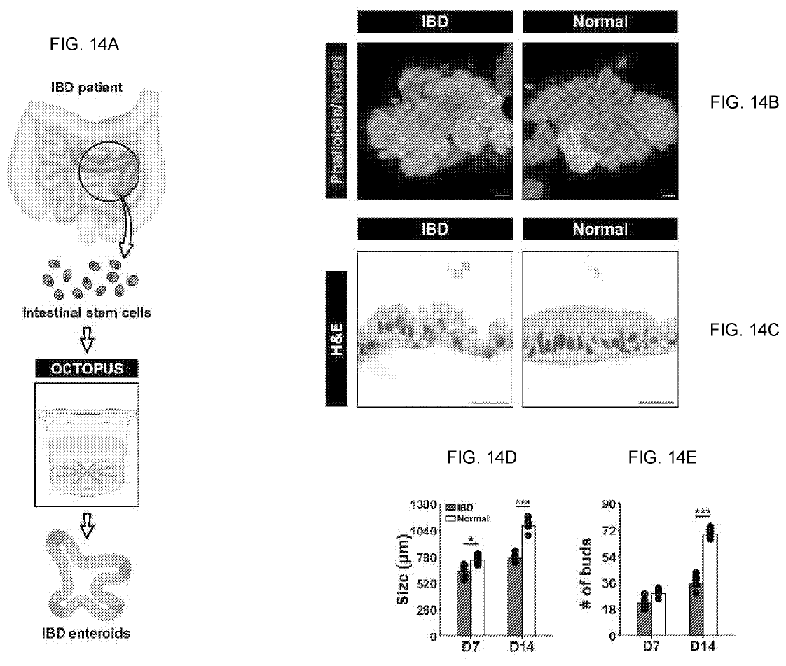

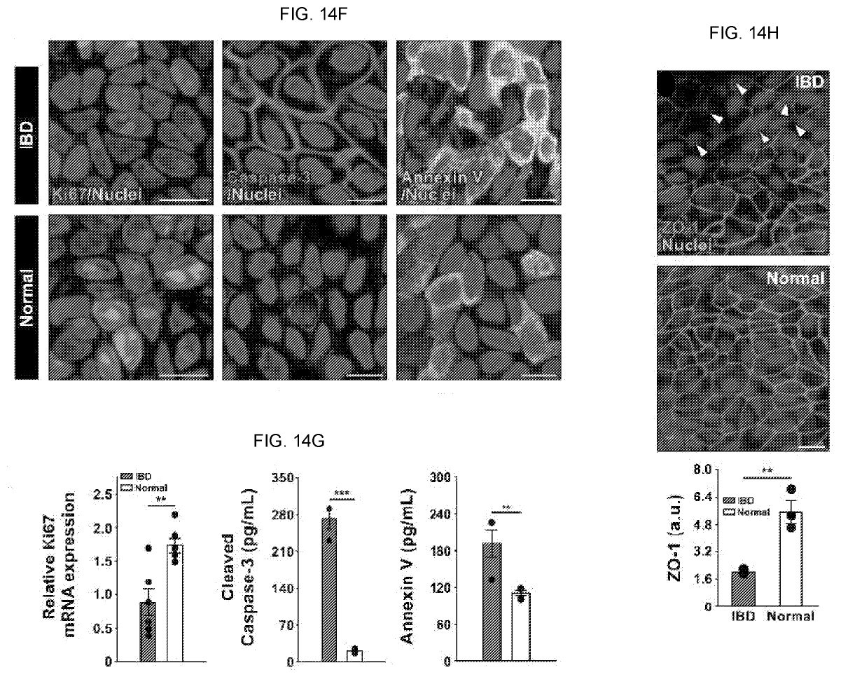

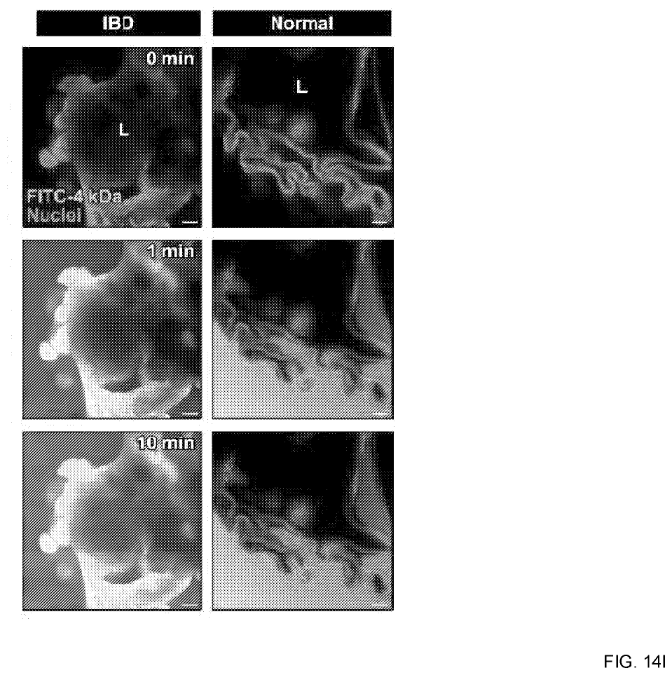

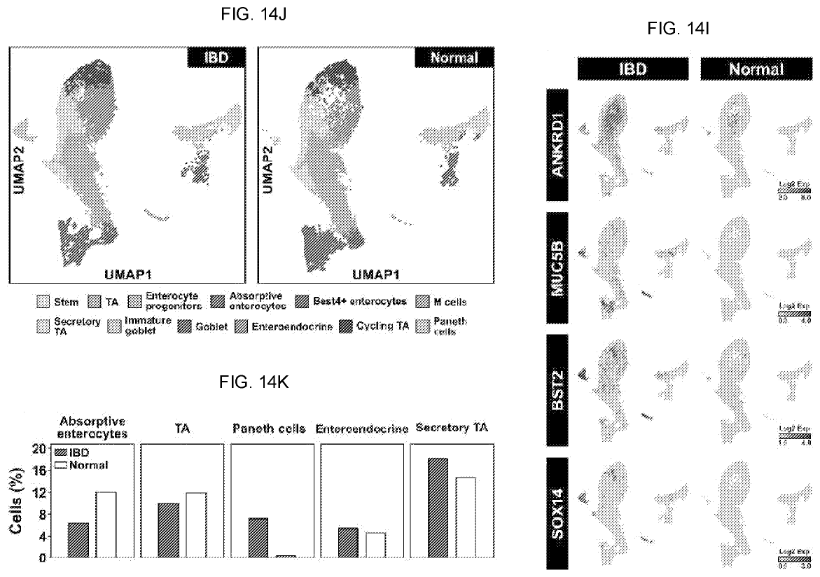

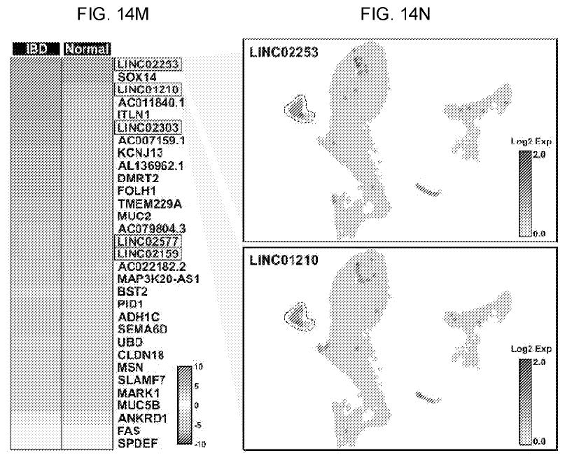

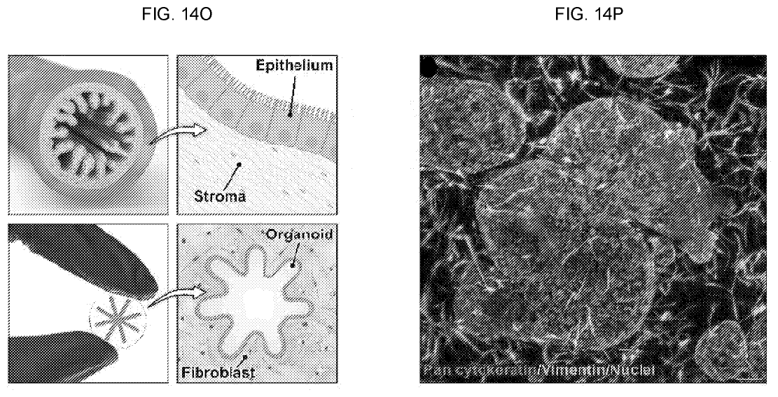

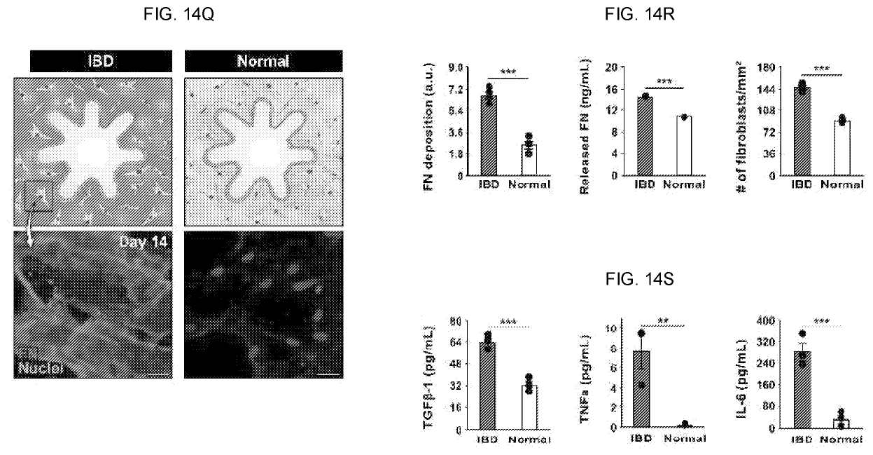

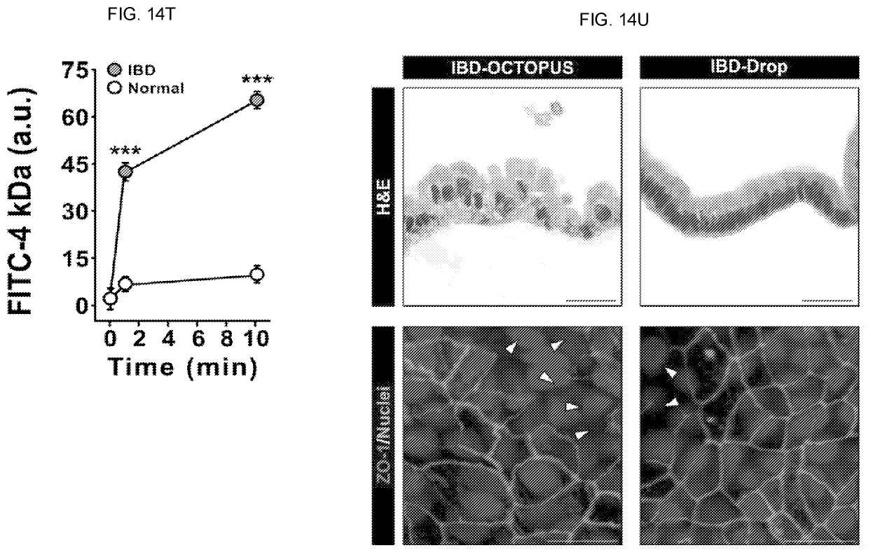

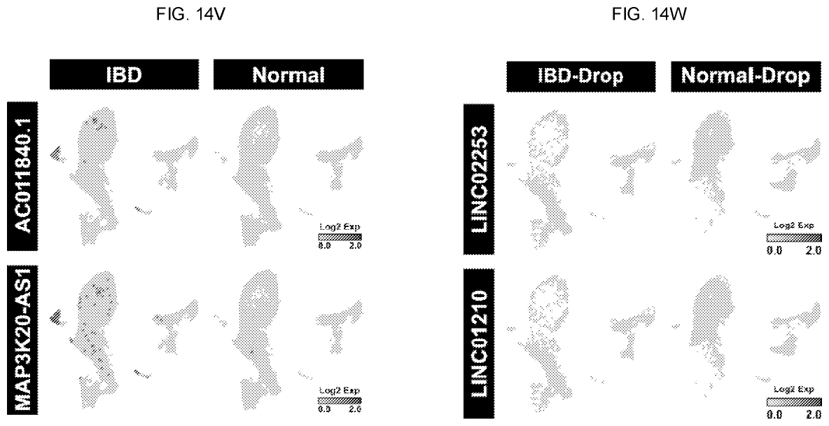

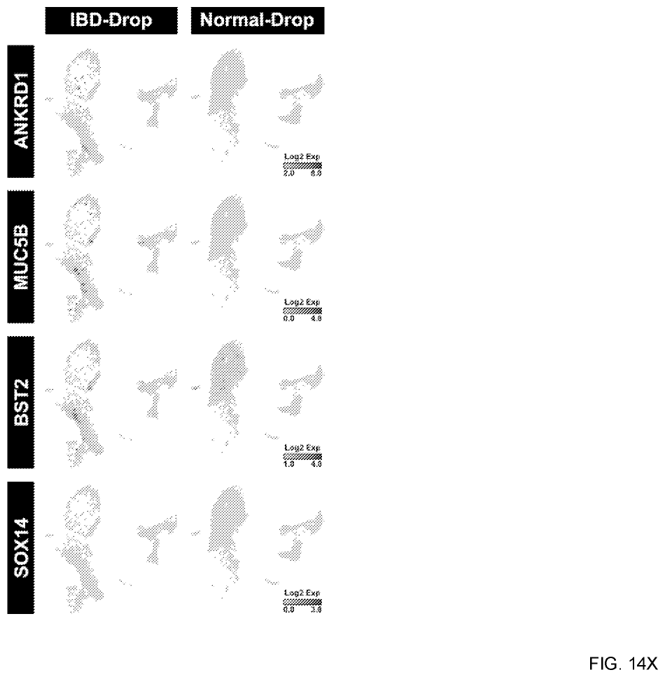

Despite advances in general understanding of IBD, modeling this complex disease remains a significant challenge. Studying IBD often relies on the use of chemical or congenic murine models that require genetic or exogenous manipulations to approximate the phenotype of human IBD. As an alternative approach, researchers have demonstrated conventional 2D and 3D culture of primary or transformed cells (e.g., Caco-2) to generate in vitro analogs of human intestinal tissues that can be subjected to externally applied inflammatory cues. To overcome the limitations of these simplified systems, new efforts are being made to use intestinal organoids to more faithfully model the pathophysiological complexity of IBD. Inspired by this emerging body of work, the feasibility of engineering human enteroids reminiscent of the dysfunctional intestinal epithelium in IBD through long-term culture of patient-derived intestinal organoids in OCTOPUS was explored (FIG. 14A).

When the cell suspension isolated from the small intestine of IBD patients were seeded into the disclosed device, they underwent self-organization over 7 days into organoids containing budding structures that appeared similar to those observed in normal enteroids derived from healthy donors (FIG. 14B). Histological analysis, however, revealed significant dissimilarities between the two groups. Normal enteroids contained properly polarized epithelial cells with small and basally-located nuclei and focally apparent apical brush border, while some areas of IBD organoids contained enterocytes with enlarged, centrally located nuclei and a higher nuclear/cytoplasmic ratio (FIG. 14C), matching histopathological findings of the intestinal epithelium in IBD patients. The IBD enteroids also grew more slowly (FIG. 14D) and formed significantly fewer buds (FIG. 14E), consistent with previous reports of defects in the villus-forming ability of the IBD intestinal epithelium. In addition to these morphological differences, the patient-derived enteroids exhibited reduced proliferative capacity and increased cell apoptosis (FIG. 14F, FIG. 14G) when compared to the normal control. The villus domain of these organoids also contained large patches of cells with decreased expression or loss of tight junctions (FIG. 14H). Due to the impaired structural integrity of the epithelium, the IBD enteroids showed compromised barrier function, as measured by permeability assay using 4-kDa fluorescein isothiocyanate (FITC)-dextran (FIG. 14I, FIG. 14T). These results demonstrate the ability of the disclosed model to recapitulate some of the key features of the defective intestinal epithelium in IBD. Of note, when the IBD organoids were generated and maintained in conventional Matrigel drops, they were unable to reproduce these disease phenotypes to the extent demonstrated in OCTOPUS (FIG. 14U).

Further analysis using scRNA-seq showed significant alterations reflecting the pathophysiological state of the IBD enteroids. One of the most noticeable changes was a nearly 50% decrease in the proportion of mature enterocytes when compared to normal enteroids (FIG. 14J, FIG. 14K). This finding approximates in vivo reports of reduced enterocyte populations in the inflamed intestinal epithelium and is indicative of dysregulated enterocytic differentiation of stem cells and/or increased apoptosis of enterocytes in our model, both of which have been described in IBD patients. Similar changes were noted in the other major subtypes of absorptive intestinal epithelial cells, such as TA cells and BEST4+ enterocytes (FIG. 14J, FIG. 14K). These results were in contrast to the expansion of some of the secretory cell populations. In particular, the proportion of Paneth cells increased from 0.37% in the normal enteroids to 7.2% in the IBD model (FIG. 14J, FIG. 14K), which is consistent with expansion and repopulation of Paneth cells that occurs in the crypt-region of the small intestine as a result of inflammation and epithelial injury. At the transcriptomic level, the sequencing data revealed substantially increased expression of IBD-associated genes in the patient-derived enteroids, including ANKRD1, MUC5B, and BST2 (FIG. 14L, FIG. 14V). Upregulated genes in this model also included the key regulators of MAPK/ERK signaling pathways, such as transcription factor SOX14 and long noncoding RNA MAP3K20-AS1 (FIG. 14L, FIG. 14V), reflecting the capacity of the organoids to emulate the nature of IBD as an inflammatory disease. Importantly, in addition to these known IBD-associated genes, several long intergenic non-protein coding (LINC) RNA genes were also discovered to b eupregulated in the patient-derived enteroids, including LINCO2253, LINC01210, LINCO2303, LINCO2577, and LINCO2159, that have not been described previously in the context of IBD (FIG. 14). Many of these markers were expressed predominantly by Paneth cells and other secretory cell types expanding in the IBD organoids (FIG. 14N). Interestingly, differential regulation of the LINC genes was absent in Matrigel drop culture (FIG. 14W). Also, upregulation of the IBD-associated genes occurred to a significantly lesser extent in patientderived enteroids cultured in Matrigel drops (FIG. 14X).

Having demonstrated the pathophysiological signatures of OCTOPUS-generated IBD enteroids at the molecular and cellular levels, their capacity to recapitulate intestinal abnormalities that develop at the tissue scale during the progression of IBD was then examined. This study focused on intestinal fibrosis, which is a common complication of IBD. The intrinsic ability of the intestine to repair wounds and restore homeostasis can be impaired by repetitive epithelial injury due to chronic inflammation in IBD. Studies have shown that persistent insults can dysregulate tissue-tissue interactions between the intestinal epithelium and the underlying stroma, leading to abnormal remodeling of the subepithelial compartment characterized by hyperproliferation of fibroblasts and excessive deposition of ECM. The goal of the study was to investigate whether the salient features of this pathophysiological fibrogenic process could be recreated in the disclosed IBD organoid model.

To this end, patient-derived IBD enteroids were co-cultured with primary human intestinal fibroblasts in the same hydrogel scaffold to generate a multicellular construct reminiscent of the intestinal epithelium and the underlying stromal tissue in vivo (FIG. 14O). Uninterrupted culture over 14 days in this mixed co-culture configuration allowed fibroblasts to spread and proliferate around the nascent organoids, eventually forming microtissues densely populated with enlarged organoids surrounded by fibroblasts (FIG. 14P). Immunostaining of the IBD constructs after 14 days of culture revealed excessive fibronectin (FN) deposition in the pericellular regions of fibroblasts (FIG. 14Q). Extracellular FN was also present in the culture of normal enteroids but its level was significantly lower (FIG. 14Q, FIG. 14R). This difference was further supported by ELISA analysis of conditioned media that showed higher concentrations of released FN in the IBD model (FIG. 14R). The patient-derived enteroids also promoted fibroblast proliferation as evidenced by almost twice as many fibroblasts in the IBD model after 14-day culture (FIG. 14R).

These findings match the general patterns of fibrotic tissue remodeling described in previous in vivo studies of the small intestine in IBD patients. Our data also suggest that spontaneous fibrogenesis in the disclosed model is driven by the diseased epithelium of the IBD enteroids. To characterize the soluble pro-fibrotic microenvironment created by these epithelial cells, the production of transforming growth factor (TGF)-β1, which is the member of the TGF-β superfamily overexpressed by the intestinal epithelium in IBD that selectively activates ECM synthesis by mesenchymal cells, was measured. As expected, TGF-β1 production was significantly upregulated in the IBD model compared to normal enteroids (FIG. 14S). Similar differences were observed in the secretion of interleukin (IL)-6 and tumor necrosis factor (TNF)-α, both of which have been shown to activate intestinal fibroblasts in IBD-associated fibrosis (FIG. 14S). Taken together, these results demonstrate the feasibility of using patient-derived intestinal organoids generated by OCTOPUS as the basis for creating more faithful and physiologically relevant models of IBD.

With rapid progress in organoid technology, there is a significant increasing demand for advanced organoid models capable of emulating more complex structure and physiological function of native organs. As a representative example, integrating vasculature into organoid cultures is emerging as an area of increasing interest in ongoing research efforts to advance the capabilities and potential of organoid technology. Vascularization of organoids is necessary for mimicking vascularity of native tissues and vascular contributions to parenchymal function but it has also been suggested as a promising strategy to improve nutrient and oxygen supply in 3D culture for enhanced organoid growth and maturation. The process of generating vascularized organoids and perfusing them in a controlled manner, however, is prohibitively complex and often requires specialized techniques and culture systems not easily accessible to non-engineers.

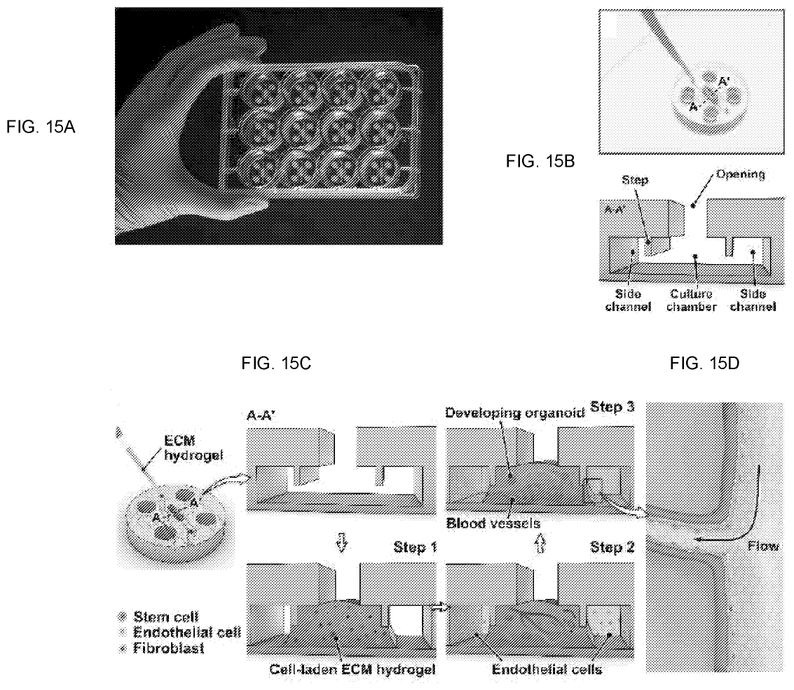

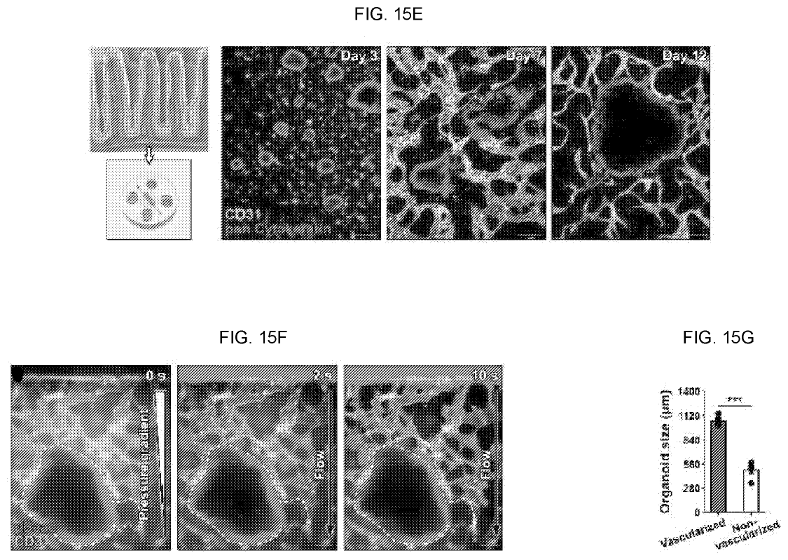

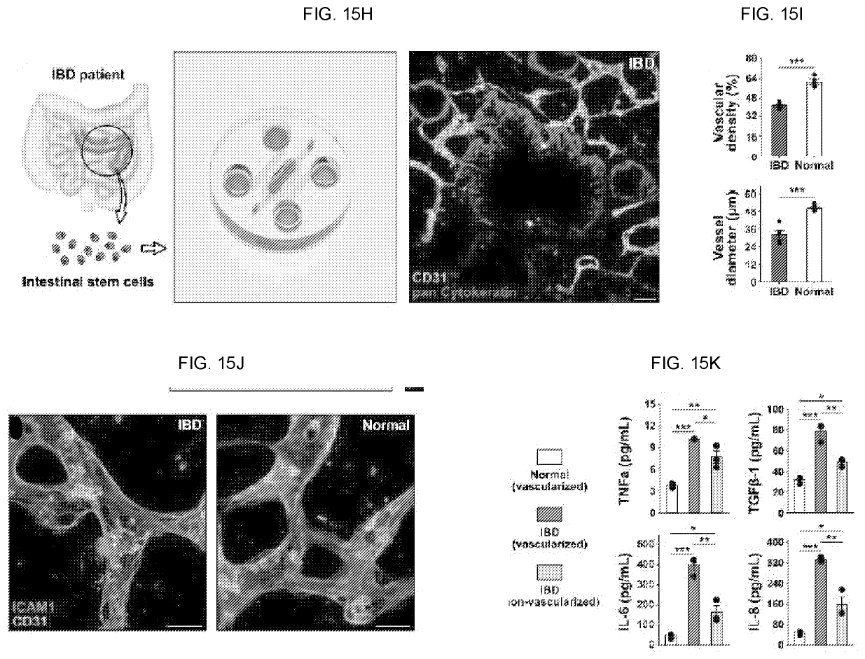

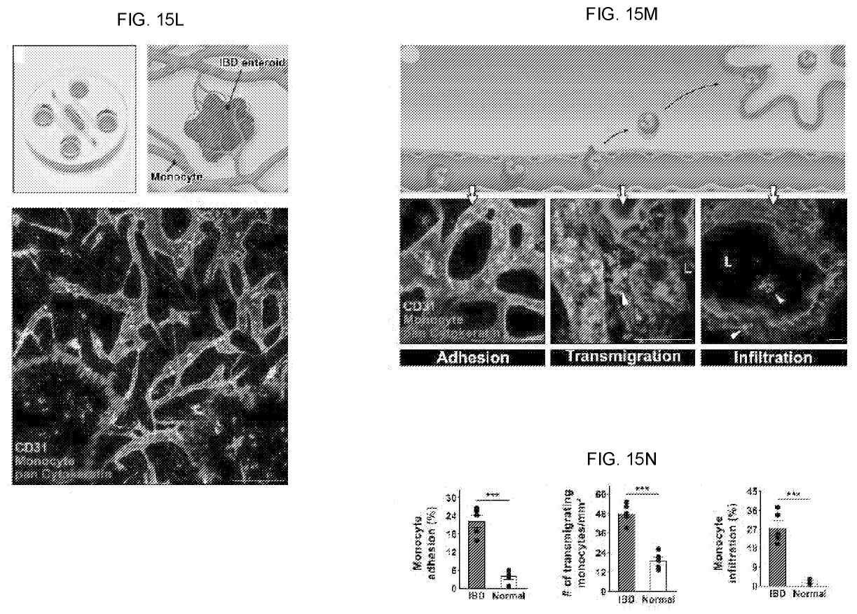

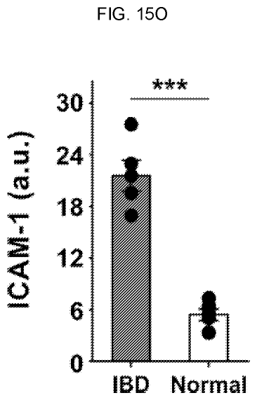

Motivated by this problem, advanced prototype of OCTOPUS that provides new capabilities to engineer vascularized, perfusable human organoids while still offering the simplicity and convenience of the original platform was created (FIG. 15). This system, termed OCTOPUS-EVO (OCTOPUS for Engineering Vascularized Organoids), was constructed by incorporating a network of microfabricated chambers into the OCTOPUS insert that are easily accessible using conventional pipettes (FIG. 15A, FIG. 15B). In an embodiment, the device consists of an open cell culture chamber flanked by two flow-through microchannels on either side of the chamber. In an example, the device consists of an open cell culture chamber with cross-sectional dimensions of 3 mm (width)×1 mm (thickness) flanked by two flow-through microchannels (1 mm×1 mm) on either side of the chamber (FIG. 15B). The side channels are individually addressable using independent access ports and divided from the cell culture chamber by a pair of microfabricated steps (FIG. 15B). To generate vascularized organoids, the culture chamber is injected with a mixture of stem cells, vascular endothelial cells, and fibroblasts suspended in an ECM hydrogel precursor solution (FIG. 15C). During this process, capillary pinning of the injected solution at the dividing steps makes it possible to physically confine the mixture in the middle lane (FIG. 15C, step 1). After gelation to form a cell-laden ECM hydrogel scaffold, the side channels are seeded with endothelial cells to form a continuous endothelial lining on the channel surface (FIG. 15C, step 2). During culture, stem cells in the hydrogel scaffold develop into organoids, while endothelial cells embedded in the same gel undergo self-assembly reminiscent of the developmental process of vasculogenesis to form a 3D network of interconnected blood vessels surrounding the developing organoids (FIG. 15C, step 3). These vessels anastomose with the endothelium in the side channels, making the vascularized organoid construct directly accessible and perfusable from the side channels (FIG. 15D).

For proof-of-concept demonstration, we seeded OCTOPUS-EVO with a mixture of fibrin and Matrigel precursors containing human intestinal cells, endothelial cells, and fibroblasts. This co-culture system supported rapid self-organization of stem cells into enteroids within 2-3 days of culture (FIG. 15E). The formation of blood vessels occurred over longer periods of time and became noticeable after 5-6 days (FIG. 15E). The intestinal organoids continued to grow in the vascularized hydrogel over the course of 12-day culture, during which the self-assembled microvasculature remained stable and closely associated with the developing enteroids without a measurable loss of structural integrity (FIG. 15E). Importantly, the entire vascularized organoid construct was perfusable as demonstrated by the flow of 1-μm fluorescent microbeads through the vascular network in the direction of applied pressure gradient across the scaffold (FIG. 15F). Another finding is that the vascularized, perfused enteroids in OCTOPUS-EVO grew more than twice as large as non-vascularized ones in OCTOPUS during the same duration of culture (FIG. 15G), illustrating the beneficial effects of perfusable vasculature on organoid growth.

Building upon the demonstration of IBD enteroids (FIG. 14), whether the EVO platform could be leveraged to vascularize the patient-derived organoids and develop a more advanced disease model that could be used for the study of vascular abnormalities and other disease processes in IBD mediated by the vasculature was explored. 12-day culture of patient-derived intestinal stem cells with endothelial cells and fibroblasts in the fibrin/Matrigel scaffold of OCTOPUS-EVO led to the formation of IBD enteroids fully enveloped by their surrounding microvasculature (FIG. 15H). Interestingly, the blood vessels in this model showed significantly reduced density and diameter compared to their counterparts formed around normal enteroids (FIG. 15E, FIG. 15I), matching vascular features of the chronically inflamed intestine in IBD. Moreover, endothelial cells in a large fraction of the vessels were seen with robust immunostaining of intercellular adhesion molecule (ICAM)-1, which was not observed in the normal group (FIG. 15J, FIG. 15O). Consistent with this finding, ELISA of vascular perfusate collected from the IBD model clearly showed considerably increased production of key inflammatory mediators known to induce endothelial activation (FIG. 15K). Importantly, the levels of most of these cytokines in the vascularized IBD enteroids were found to be significantly higher than those in the non-vascularized IBD model (FIG. 15K), suggesting improved transport of released cytokines out of the organoid constructs and/or potential contributions of vascular endothelial cells to the pro-inflammatory milieu of the IBD enteroids.

Finally, the observation of endothelial activation in the vascularized IBD enteroids led to investigation of whether vascular perfusability of the dislcosed model could be exploited to simulate the recruitment of blood-borne immune cells in IBD. In vivo evidence has established a marked increase in the recruitment of circulating blood monocytes to the intestinal mucosa as one of the key immunological events during the development of IBD. Indeed, the disclosed IBD model perfused with human peripheral blood monocytes showed a large number of cells in the enteroid-associated blood vessels, as well as in the lumen of the enteroids (FIG. 15L). The monocytes in the vessels remained firmly adherent to the endothelial lining even in the presence of intraluminal flow (left panel, FIG. 15M). Closer examination of the construct revealed that some of these adherent monocytes migrated across the endothelium into the perivascular space (middle panel, FIG. 15M). Also captured in this model were monocytes undergoing transmigration across the intestinal epithelium into the lumen of the enteroids (right panel, FIG. 15M). These complex events that reproduce the sequential steps of monocyte recruitment in vivo were observed in much fewer cells when monocytes were infused into vascularized enteroids derived from healthy donors (FIG. 15A).

This data altogether provide the proof-of-concept of OCTOPUS-EVO and demonstrate its potential as an accessible in vitro platform to engineer vascularized, perfusable organoids that can expand the capabilities of conventional organoid cultures.

In response to the increasing need for new technologies for organoid research, the present disclosure describes a microengineered platform to reconfigure the three-dimensionality of conventional organoid culture. OCTOPUS provides a simple yet effective means to address the problem of limited nutrient supply inherent in 3D culture. By enabling controlled production of open 3D culture scaffolds with significantly decreased thickness, this system serves to reduce the distance and spatial variability of nutrient and oxygen diffusion to growing organoids. In comparison to conventional Matrigel drop culture, this design makes it possible to engineer a more uniform, unrestricted soluble microenvironment beneficial for long-term culture of organoids. The improved mass transport characteristics due to significantly reduced diffusion limitations also decrease the effective culture volume of the disclosed system, which is an inverse measure of the ability of cells to process and control their environment during culture. As a result, stem cells and organoids in OCTOPUS have better control over their local microenvironment during development. Data described herein show that these desirable features of OCTOPUS can increase the size and maturity of organoids beyond what is achievable using conventional techniques and may enable the production of more realistic multicellular constructs for in vitro modeling of organogenesis and disease development.

OCTOPUS enables uninterrupted, continuous organoid culture for extended periods of time. As shown by scRNA-seq of the human enteroid model, doubling the duration of uninterrupted culture using OCTOPUS greatly promoted enterocyte differentiation in organoids to generate a more physiological intestinal epithelium that contained substantially larger numbers of functionally mature enterocytes, as compared to Matrigel drop.

Although the ability to support continuous long-term culture is a key advantage of OCTOPUS, the data herein reveal additionally desirable features of organoid development in the disclosed system. After 7 days of culture, for example, the size of intestinal organoids and the expression of virtually every marker of epithelial maturation were significantly greater in OCTOPUS. ScRNA-seq analysis provided further evidence that human enteroids in OCTOPUS more faithfully recapitulated the cellular heterogeneity of the native intestinal epithelium, as well as the relative abundance of differentiated cell types and their physiological gene expression profiles, when compared to those cultured in conventional Matrigel drops for the same amount of time. These results suggest that OCTOPUS is capable of accelerating the growth and maturation of organoids at the early stage of development.

By leveraging these capabilities, this work demonstrated the feasibility of developing a specialized organoid model that can recapitulate morphological, functional, and transcriptional characteristics of the diseased human intestinal epithelium in IBD. Interestingly, many of the pathophysiological alterations observed in this model did not occur in Matrigel drop culture of patient-derived organoids. Another observation of the OCTOPUS IBD model was increased expression of several long noncoding RNAs (lncRNAs). This finding may have important implications for the emerging investigation of lncRNA biology in IBD. Recent evidence suggests active involvement of lncRNAs in mediating key disease processes of IBD associated with epithelial permeability, apoptosis, and inflammation. To this end, the scRNA-seq data reveal a set of lncRNAs that have not been implicated in IBD. LINC02159 and LINC02577 are among these genes that have been shown to play a role in tumorigenesis by promoting the proliferation of colorectal cancer cells. LINC01210 is another lncRNA previously described as a regulator of colorectal and ovarian cancer cell proliferation and invasion.

Inclusion of intestinal fibroblasts in this model permitted in vitro reproduction of intestinal fibrosis. Unlike previous demonstrations of organoid-based fibrosis models generated by treatment with exogenous fibrogenic factors (e.g., TGF-β), the disclosed co-culture system spontaneously developed fibrosis without external input to recapitulate the key features of abnormal matrix remodeling described in the small intestine of IBD patients. This finding supports the general notion of the diseased or persistently injured epithelium as the driver of pathophysiological organ fibrosis that can activate effector cells in the subepithelial compartment. Thus, the disclosed system may provide a simple yet enabling platform for organoid-based mechanistic investigation of dysregulated fibrogenesis in the intestine. Given that the biological processes underlying the development of fibrosis are conserved across organs, the same device and organoid culture techniques may be applicable to studying fibrotic diseases in other organs.

The demonstration of organoid vascularization highlights the advanced capabilities and potential of OCTOPUS. OCTOPUS-EVO enabled the concurrent, spontaneous process of organogenesis and vasculogenesis in the same culture scaffold to produce vascularized, perfusable human enteroids that can recreate the vascular-parenchymal interface and more complex physiological responses of native organs. Researchers have recently introduced techniques for organoid vascularization, including in vivo transplantation of organoids into vascular-rich organs such as the brain, kidney, lung, and pancreas, but generating such constructs with controlled vascular perfusion in vitro remains a major challenge. OCTOPUS-EVO provides an accessible means to tackle this challenge and increase the complexity of organoid models at the convenience and simplicity of conventional 3D culture without requiring specialized engineering systems. Vascularized enteroids in the disclosed device had significantly larger size compared to non-vascularized ones, supporting the notion that organoid vascularization is a promising strategy to facilitate organoid growth. Presumably, vascularization of the culture scaffold increases nutrient and oxygen supply to permit more efficient and rapid organoid development. Based on a large body of evidence demonstrating endothelial interactions with parenchymal tissues, it is also possible that biological crosstalk between the vasculature and organoids may be responsible for increased organoid growth.

While OCTOPUS represents considerable changes to the design of traditional organoid models, the implementation of this system does not require any modification of established culture protocols and workflow, nor does it rely on specialized equipment or personnel. Essential to this advantage is the design of OCTOPUS as a ready-to-use and easily-accessible culture insert that is directly compatible with standard well plates and laboratory infrastructure. As exemplified by the disclosed intestinal models, generating mature organoids in OCTOPUS can readily be accomplished in traditional laboratory settings based on materials and experimental procedures commonly used in conventional techniques. This is an important aspect of the disclosed methods that makes OCTOPUS an immediately deployable and readily accessible culture platform, which may contribute to rapid dissemination of the technology for widespread use.

### Methods

The below described methods were applied, as appropriate, for each of the above-described Examples.

For organoid cultures, cryopreserved mouse intestinal organoids (70931, STEMCELL Technologies, Canada) and cryopreserved mouse hepatic progenitor organoids (70932, STEM-CELL Technologies, Canada) were used. Intestinal and liver organoids were cultured in 24-well plates according to the manufacturer's protocols using IntestiCult™ organoid growth medium (06005, STEMCELL Technologies, Canada) and Hepaticult™ organoid growth medium (06030, STEM-CELL Technologies, Canada), respectively. To briefly explain, previously existing Matrigel drop was dissolved by incubating in Dispase. Upon incubation for 30 minutes, organoids were physically dissociated into single cell suspension and then transferred to a 15 ml falcon tube and centrifuged at 290×g to obtain stem cell pellet. 100 μl of complete organoid growth medium was then added to the pellet. After 100 μl of cold Matrigel was added, the suspension was gently pipetted up and down 10 times for thorough mixing. Using a pre-wetted 200 μl tip, 50 μl of the organoid/Matrigel mixture was injected into a 24-well plate to form Matrigel drop. The drop-containing well plates were then incubated at 37° C. and 5% CO2 for 10 minutes to allow gelation of Matrigel. Upon completion of this step, 750 μl of pre-warmed organoid growth medium was added to each well. Organoids were passaged every 5-7 days in fresh Matrigel until use as recommended by the manufacturer.

Regarding human enteroid lines, enteroid lines generated from terminal ileum were provided by the Children's Hospital of Philadelphia Gastrointestinal Epithelium Modeling Program under an Institutional Review Board-approved protocol (13042). All parents of patients provided written informed consent. Enteroid lines were generated. Briefly, two biopsy tissue fragments were rinsed 3 times in 1 ml cold sterile PBS, then incubated in cold chelation buffer for 30 minutes on a turntable in a cold room, followed by mechanical dissociation (scraping) of epithelial layer. The fragments were strained through a 100 μm strainer to deplete the villi and resuspended in 80% Matrigel, then seeded at the density of crypts per 30 μL drop. The droplets solidified at 37° C. for 30 minutes, and 500 μl human IntestiCult (STEMCELL Technologies; complete when supplemented with Penicillin-Streptomycin (Gibco)) was added per well. Y-27632 (SelleckChem; final conc. 10 μM) was added to culture medium at seeding only.

Regarding maintenance and passage of human enteroids, enteroid media was changed three times per week. On day 14, the cultures were passaged and/or cryopreserved in CryoStor CS-10 (STEMCELL Technologies). To passage, the Matrigel droplet was dislodged by pipetting up and down through a P1000 tip and transferred into a 1.5 ml microfuge tube, followed by centrifugation and washing with ice-cold HBSS. Enteroids were mechanically dissociated into fragments by pipetting 10 times through a P200 tip placed on top of a P1000 tip, followed by centrifugation. The pellet was reconstituted in 80% Matrigel and seeded as 30 μl drops at a split ratio of 1:4. Subsequent cultures are ready for passage and/or cryopreservation on day 7.

Regarding formation of 3D organoid constructs in OCTOPUS, standard 24-well plates containing OCTOPUS inserts were first sterilized by exposure to ultraviolet (UV) light (Electro-lite ELC-500) for 30 minutes. Subsequently, the culture chambers in OCTOPUS were filled with 2 mg/ml (w/v in 10 mM Tris-HCl buffer, pH 8.5) of dopamine hydrochloride solution at room temperature (RT) for 2 hours to form a surface coating for enhanced adhesion of Matrigel to PDMS. The poly(dopamine) (PDA)-treated devices were kept sterile until use. To form organoids in the disclosed device, the pellets were made first. To this end, existing Matrigel was dissolved by incubating Matrigel drop in Dispase. Cells were then transferred to a 15 mL falcon tube and centrifuged at 290×g to obtain a stem cell pellet. Then, 100 μl of complete IntestiCult™ organoid growth medium was added to the pellet. After 100 μl of cold Matrigel was added, the suspension was gently pipetted up and down 10 times for thorough mixing. For human intestinal enteroids, a cell pellet can be resuspendeded in 80% Matrigel. Using a pre-wetted 200 μl tip, 100 μl of the organoid/Matrigel mixture was injected into OCTOPUS through the injection port. The OCTOPUS-containing well plates were then incubated at 37° C. and 5% CO2 for 10 minutes to allow gelation of Matrigel. Upon completion, 750 μl of pre-warmed IntestiCult™ organoid growth medium was added to each well. The OCTOPUS plates were maintained in cell culture incubators at 37° C. and 5% CO2. During long-term culture, media exchange was conducted every other day.

For measurement and quantification of caspase-3, annexin V, TNFa, TGFβ-1, IL-6 and IL-8 in the human enteroids, conditioned media were collected on day 14 of culture and analyzed using cleaved caspase-3 (Asp175) ELISA kit (ab220655, abcam), human annexin V ELISA kit (ab223863, abcam), human TNF alpha ELISA kit (ab181421, abcam), human TGF beta 1 ELISA kit (ab100647, abcam), human IL-6 ELISA kit (ab178013, abcam), and human IL-8 ELISA kit (ab214030, abcam). Each assay was performed following the manufacturer's protocol. Briefly, 100 μl of a standard solution or sample media was added to each well. After 2-hour incubation, the well was washed 5 times with 300 μl of manufacturer-provided wash buffer and incubated with secondary antibody for 1 hour. After washing, 100 μl of TMB substrate was added to each well and incubated for 20 minutes in the dark. Finally, 100 μl of stop solution was added to each well, and the plate was measured in a plate reader (M200, Tecan, Switzerland). For all ELISA assays, we used a multimode plate reader (M200, Tecan, Switzerland) to measure the optical density of samples. A standard curve was generated by plotting the mean optical density and concentration for each standard using a four-parameter logistic curve fitting method. Sample measurements were converted to target concentrations using the standard curves.

To model intestinal fibrosis as a complication of IBD, human intestinal stem cells were co-cultured with 1×10⁶ cells/ml of primary human intestinal fibroblasts in Matrigel (356255, Corning, USA). This cell-containing hydrogel solution was injected into the device to form microtissue constructs in the organoid culture chambers. After gelation for 15 minutes in a regular cell culture incubator, 750 μl of IntestiCult™ organoid growth medium (06010, STEMCELL Technologies, Canada) was added into each well and maintained for 14 days to induce intestinal organoid development and fibroblast proliferation. During this period, the media were replenished every other day.

Regarding the formation of vascularized human enteroids in OCTOPUS-EVO, the fully assembled device was sterilized before cell culture by exposing it to ultraviolet (UV) light (Electro-lite ELC-500) for at least 30 minutes. To engineer vascularized organoids in OCTOPUS-EVO, 20 μl of cell suspension solution containing fibrinogen (5 mg/ml; F8630, Sigma), thrombin (1 U/ml; T7513, Sigma), aprotinin (0.15 U/ml; A1153, Sigma), human intestinal stem cells, primary human umbilical vein endothelial cells (HUVECs) (5×10⁶ cells/ml), and primary normal human lung fibroblasts (NHLFs) (1×10⁶ cells/ml) was prepared and injected it into the open cell culture chamber through its inlet access port. The device was then left in a cell culture incubator at 37° C. and 5% CO² for 30 minutes. Upon gelation, IntestiCult media mixed with EGM-2 endothelial media were added to the medium reservoirs and the side microchannels. Following the formation of cell-laden hydrogel construct, the side microchannels were incubated with a fibronectin solution (25 μg/ml in PBS; 356008, Corning) for 2 hours at 37° C. to create ECM coating on the channel surface. Then, the channels were washed once with IntestiCult/EGM-2, and 10 μl of HUVEC suspension (1×107 cells/ml) was introduced into both channels. The seeded cells were allowed to attach to the channel surface over a period of 1 hour. Upon 1 hour incubation, pre-warmed media was added to each medium reservoir. This culture condition allowed the endothelial cells to form confluent monolayers on the surface of the side channels and the hydrogel scaffold to induce anastomosis between the endothelial lining and the self-assembled vasculature in the hydrogel.

To examine the viability of organoids, Live/Dead™ Viability/Cytotoxicity Kit was used for mammalian cells (L3224, ThermoFisher Scientific, USA). For this assay, a mixture of calcein AM (2 μM) and ethidium homodimer-1 (4 μM) in live-cell imaging solution was introduced into the OCTOPUS-containing wells and incubated at RT for 30 minutes. Subsequently, the samples were washed with phosphate-buffered saline (PBS) three times, after which the labeled cells were examined using a laser scanning confocal microscope (LSM 800, Carl Zeiss, Germany). For quantitative analysis, the fraction of healthy and necrotic organoids was calculated from fluorescence generated by calcein AM and ethidium homodimer-1, respectively. In each device, 30 organoids were used for the analysis.

To examine the spatiotemporal patterns of diffusion in OCTOPUS and hydrogel drops, FITC-dextran, either 4 kDa FITC-dextran or 70 kDa FITC-dextran (FD70S-100MG, Sigma, USA), was used as a fluorescent tracer for visualization. For this assay, the organoid culture medium was replaced with a FITC-dextran solution (50 μg/ml in PBS). Dextran diffusion was monitored and visualized using a laser scanning confocal microscope (LSM 800, Carl Zeiss, Germany). Time-lapse images were acquired for 120 minutes and processed using ZEN software (Zeiss, Germany) to measure temporal changes in fluorescence intensity at defined locations within the hydrogel scaffolds.

To detect proliferating cells within the intestinal organoids, EdU assay/EdU staining proliferation kit-iFluor 647 (ab222421, abcam, USA) was used. Briefly, the organoids were incubated with a EdU solution (20 μM in medium) for 3 hours under normal culture conditions (5% CO2 at 37° C.). The organoids were then washed twice with PBS, fixed in 4% formaldehyde, and permeabilized using a permeabilization buffer, according to the manufacturer's protocol. The samples were stained with iFluor 647 azide dye and visualized using a confocal microscope (LSM 800, Carl Zeiss, Germany).

For calcium imaging, the organoid media were removed from the culture wells, and the organoid constructs were washed once in live-cell imaging solution (LCIS). The organoids were then loaded with Fluo-4 calcium imaging solution (F10489, ThermoFisher Scientific, USA) prepared according to the manufacturer's protocol. The samples were incubated at 37° C. for 30 minutes, which was followed by another 30-minute incubation at room temperature. Subsequently, the Fluo-4 solution was removed, and the organoids were washed once with LCIS. All samples were kept in fresh LCIS until use. An inverted epi-fluorescence microscope (Axio Observer D1, Zeiss, Germany) was used to visualize calcium staining of organoids upon stimulation with 100 μM of ATP (A1852, Sigma, USA) and 50 mM of glucose (G7021, Sigma, USA).

For ratiometric analysis of changes in Ca2+ levels, fluorescence intensity for each or-ganoid was measured during an experiment, and values were normalized by their resting intensities using the equation below.

ΔCa²⁺=(F−Fᵣₑₛₜ)/Fᵣₑₛₜ (1)

To analyze GLP-1 and mucin 2 secretion from the intestinal organoids, the media in the wells were collected on days 5, 7, and 10 of culture. Multi-species GLP-1 total ELISA kit (EZGLP1T-36K, Millipore Sigma, USA), Glucagon-like peptide-1 (active) ELISA kit (EGLP-35K, Millipore Sigma, USA), and MUC2 ELISA kit (ABIN6730976, antibodies-online Inc, USA) were used to measure the concentrations of GLP-1 total, GLP-1 active, and mucin 2, respectively. Each assay was performed following the manufacturer's protocol. Briefly, 100 μl of a standard solution or sample media was added to each well. After 2-hour incubation, the well was washed 5 times with 300 μl of manufacturer-provided wash buffer and incubated with secondary antibody for 1 hour. After washing, 100 μl of TMB substrate was added to each well and incubated for 20 minutes in the dark. 100 μl of stop solution was added to each well, and the plate was measured in a plate reader (M200, Tecan, Switzerland).

For the analysis of fibronectin production in the intestinal fibrosis model, a mouse fibronectin ELISA kit (ab108849, abcam, USA) was used. The media in the wells were collected at specified time points and assayed using manufacturer-provided protocols. First, 50 μl of standard or device-collected samples were added into each well and incubated for 2 hours at room temperature. Subsequently, the wells were washed 5 times with 300 μl of wash buffer solution and then incubated with fibronectin antibody for 1 hour. After washing, the streptavidin-peroxidase conjugate was added to each well, incubated for 30 minutes, and washed again. The samples were incubated with 50 μl of chromogen substrate for 10 minutes, followed by the introduction of 50 μl of stop solution.

For all ELISA assays, a multimode plate reader (M200, Tecan, Switzerland) was used to measure the optical density of samples. A standard curve was generated by plotting the mean optical density and concentration for each standard using a four-parameter logistic curve fitting method. Sample measurements were converted to target concentrations using the standard curves.

For certain co-culture demonstrations, primary mouse intestinal fibroblasts (mIFs) and primary human umbilical vein endothelial cells (HUVECs) were used. For initial expansion from cryostorage, mIFs and HUVECs were cultured in 75 cm² flasks according to the manufacturer's protocols using complete fibroblast medium (M2267, Cell Biologics, USA) and endothelial cell growth medium (EGM)-2 (CC-3162, Lonza, Switzerland) supplemented with growth factors, respectively. Primary mIFs and primary human intestinal fibroblasts were used for modeling intestinal fibrosis. All cells were between passages 3 and 6.

To form an intestinal fibrosis model suitable for, e.g. drug testing, in OCTOPUS, mouse intestinal stem cells were mixed with 1×10⁶ cells/ml of mouse intestinal fibroblasts in Matrigel (356255, Corning, USA). This cell-containing hydrogel solution was injected into the device to form microtissue constructs in the organoid culture chambers. After gelation for 15 minutes in a regular cell culture incubator, 750 μl of IntestiCult™ organoid growth medium (06005, STEMCELL Technologies, Canada) was added into each well and maintained for 5 days to induce intestinal organoid development and fibroblast proliferation. During this period, the media were replenished every other day. To induce fibrosis, 1 ng/ml of TGF-β (T5050, Sigma, USA) was added to the culture wells on day 5 and maintained for additional 7 days. Drug administration occurred on day 12 using commercially available Pirfenidone (P1871, TCI America, USA) and Nintedanib (S1010, Selleckchem, USA) at specified concentrations. The fibrosis model was treated with drugs for 48 hours, after which changes in its fibrotic phenotype were analyzed using the methods described herein.

To examine oxygen diffusion in OCTOPUS and Matrigel drops, Dichloritis(1,10-phenanthroline)ruthenium(II) hydrate (Ru(phen)3) (Sigma, Cat. #343714) was used as an oxygen indicator—dissolved oxygen molecules induce quenching of this fluorescent dye. Briefly, 15 μl of Ru(phen)3 (2 mM) and 270 μl of Matrigel were mixed with 15 μl of sodium sulfite (200 mM) (Sigma, Cat. #S0505), which was used to remove remaining aqueous oxygen in Matrigel. The mixture was then used to generate 3D tissue constructs in OCTOPUS and Matrigel drop. Oxygen diffusion was monitored by confocal microscopy, during which images were acquired at defined time intervals and locations within the constructs. The captured images were analyzed using ImageJ to measure spatiotemporal changes in fluorescence intensity.

To test the perfusability of the microengineered vascular network, fluorescently labeled 1-μm microbeads (FluoSpheres; F-8815, ThermoFisher) were used as flow tracers. To generate flow through the vasculature, media in the reservoirs was aspirated and a bead solution was inserted into one of the side microchannels. This configuration created a gradient of hydrostatic pressures across the hydrogel scaffold and provided driving force for the flow of microbeads through the vessels. Vascular perfusion was monitored and visualized using a laser scanning confocal microscope (LSM 800, Carl Zeiss, Germany).

To perform a monocyte infiltration assay, human peripheral blood monocytes were obtained from the Human Immunology Core at the University of Pennsylvania. To test endothelial adhesion of monocytes in the disclosed system, cells were labeled with a fluorescent dye (CellTracker Deep Red, ThermoFisher) and suspended in IntestiCult/EGM-2 media at the final concentration of 3×10⁶ cells/ml. The cells were then injected into the vessels through one of the side microchannels and allowed to flow through the vasculature for 24 hours in a cell culture incubator. At the completion of perfusion, the device was washed with DPBS three times and examined to analyze the number of adhered, transmigrated, and infiltrated monocytes.

Atomic force microscopy (AFM, MFP-3D-BIO, Asylum) was used to measure the stiffness of hydrated microtissues in the intestinal fibrosis models. A gold-coated cantilever (SCONT tip, NANOSENSORS) with a spring constant of 14.58 pN/nm and a pyramid indenter was used to obtain force-indentation curves. The tissue samples in the open chambers were used directly without any modification. For the AFM measurement, the OCTOPUS insert containing microtissue was removed from the plate and mounted on the instrument. After wetting the microtissue with a drop of PBS, its mechanical property was measured with the scanning probe. Young's modulus was calculated from the force indentation data using the Atomic J software.

For immunofluorescence staining, cells in OCTOPUS were washed with PBS twice, fixed with 4% paraformaldehyde (Electron Microscopy Sciences, USA) for 15 minutes at room temperature, and washed again twice with PBS. The cells were then permeabilized with 0.1% Triton X-100 (Sigma) in PBS for 3 minutes and exposed to blocking buffer composed of PBS and 3% bovine serum albumin (BSA; Sigma) for overnight at 4° C. After washing with PBS twice, the cells were immunostained for actin filaments (Phalloidin-iFluor 488 Reagent, ab176753, 1:1000, abcam, USA; Phalloidin-iFluor 594 Reagent, ab176757, 1:1000, abcam, USA), mature epithelial cells (anti-EPCAM antibody, ab71916, 1:250, abcam, USA; anti-HNF-4-alpha antibody [K9218]-ChIP Grade, ab41898, 1:500, abcam, USA), stem cells (anti-Ki67 antibody, ab15580, 1:1000, abcam, USA; Lgr5 monoclonal antibody, MA5-25644, 1:1000, ThermoFisher Scientific, USA), enterocytes (anti-Villin antibody [3E5G11]-N-terminal, ab201989, 1:500, abcam, USA), goblet cells (anti-MUC2 antibody, ab90007, 1:200, abcam, USA), enteroendocrine cells (anti-Somatostatin antibody [M09204], ab30788, 1:100, abcam, USA), peptide transporter 1 (anti-SLC15A1/PEPT1 antibody, ab203043, 1:100, abcam, USA), glucose transporter 1 (anti-Glucose transporter GLUT1 antibody [SPM498], ab40084, 1:250, abcam, USA), endothelial cells (anti-CD31 antibody [JC/70A] (Alexa Fluor® 488), ab215911, 1:100, abcam, USA), alpha smooth muscle actin (recombinant anti-alpha smooth muscle Actin antibody [E184], ab32575, 1:500, abcam, USA), fibronectin (anti-Fibronectin antibody [IST-9], ab6328, 1:200, abcam, USA), alpha smooth muscle actin (recombinant anti-alpha smooth muscle Actin antibody [E184], ab32575, 1:500, abcam, USA), fibronectin (anti-Fibronectin antibody [IST-9], ab6328, 1:200, abcam, USA), cleaved caspase-3 (anti-cleaved caspase-3 antibody [E83-77], ab32042, 1:200, abcam, USA), annexin V (anti-annexin V/ANXA5 antibody [EPR3980], ab108194, 1:500, abcam, USA), or ICAM1 (anti-ICAM1 antibody [EPR24639-3], ab282575, 1:500, abcam, USA). After overnight incubation with primary antibody at 4° C., the cells were washed twice with PBS and incubated with secondary antibody (Goat anti-Rabbit IgG H&L (Alexa Fluor® 488), ab150077, 1:1000, abcam, USA; Goat anti-Mouse IgG H&L (Alexa Fluor® 488), ab150113, 1:1000, abcam, USA; Goat anti-Mouse IgG H&L (Alexa Fluor® 594), ab150116, 1:1000, abcam, USA; Goat anti-Rabbit IgG H&L (Alexa Fluor® 594), ab150080, 1:1000, abcam, USA) overnight at 4° C. For nuclear staining, DAPI (D1306, Ther-moFisher Scientific, USA) diluted at 1:1000 was used. Fluorescence images of the stained cells were acquired using a laser scanning confocal microscope (LSM 800, Carl Zeiss, Germany) and processed using ZEN software (Zeiss, Germany) and ImageJ software.

For hematoxylin and eosin (H&E) staining of human enteroid, organoids were washed with cold PBS and fixed with 4% paraformaldehyde (Electron Microscopy Sciences, USA). The organoids were then resuspended in embedding gel composed of 2% bacto-agar and 2.5% gelatin and transferred as a droplet onto the embedding rack. After the gel was solidified for 30 minutes, the organoid embedded gel was placed in the pre-labeled tissue cassette and submerged in 70% ethanol. The slides containing paraffin sections were deparaffinized and rehydrated by immersing the slides sequentially into 3× Xylene, 2×100% ethanol, 95-95-80-70% ethanol, and distilled water. Then, the slides were immersed in 10 mM citric acid buffer (pH 6.0) and incubated in microwave oven for 15 minutes. After gently rinsing the slides, tissue sections were blocked with protein blocking agent. To perform H&E staining, the slides were immersed in Hematoxylin followed by rinsing with deionized water. The slides were further immersed in Eosin for 30 seconds and dehydrated in 95% ethanol-100% ethanol-xylene solutions. Tissue sections were covered with coverslip slides using Permount and stored until analysis.

Quantitative RT-PCR analysis was performed as follows. For RNA isolation, organoids were harvested by dissolving Matrigel including organoids with cold PBS. Following centrifugation at 300×g for 5 minutes at 4° C., the supernatant was removed and the pelleted organoids were resuspended in 350 μL of RLT buffer (QIAGEN). Total RNA was isolated using the RNeasy Mini Kit (QIAGEN) according to the manufacturer's instructions. cDNA was synthesized using iScript cDNA Synthesis Kit (Bio-Rad) following the manufacturer's instructions. Quantitative RT-PCR was performed using TaqMan® gene expression assays.

For single-cell sequencing analysis, collected organoids were incubated in trypsin for 10 minutes at 37° C. and passed through a 20-μm cell strainer. Isolated single cells were re-suspended at a density of 700 live cells/μl in DMEM with 5% fetal bovine serum (FBS). The cells were then stained with trypan blue to check their viability and counted under the microscope twice to determine the average cell concentration.

Single-cell suspension for each organoid sample was loaded onto a separate channel of a Chromium 10× Genomics Single Cell 3′ Reagent Kit v2 library chip (10× Genomics) according to the manufacturer's protocol. RNA transcripts from single cells were uniquely barcoded and reverse-transcribed. cDNA sequencing libraries were prepared according to the manufacturer's protocol (10× user guide for library prep) and sequenced on an Illumina NovaSeq 6000 using an S1 100 cycles flow cell v1.5. Library quality control was done using Agilent TapeStation for sizing (bp) and KAPA qPCR for concentration (nM). Raw sequence reads data were processed using the CellRanger pipeline (10× Genomics, v.5.0.0) for demultiplexing and aligned to the human genome GRCh38 transcriptome. Sample data was aggregated using the CellRanger aggr pipeline and libraries were normalized for sequencing depth across the sample set. A total of 5 organoid sample count matrices were merged together for cell type identification and direct comparisons.

Regarding statistical analysis, the sample size for each experiment was determined on the basis of a minimum of n=3 independent devices for each experimental group. Data were analyzed with Student's t-test using OriginLab (OriginLab Corporation, USA) and presented as mean±S.E.M. Statistical significance of the obtained data was attributed to values of *P<0.05, **P<0.01, and ***P<0.001 as determined by one-way ANOVA analysis.

### Embodiments

The following Embodiments are illustrative only and do not limit the scope of the present disclosure or the appended claims.

Embodiment 1. A device for culturing organoids, comprising: an access port configured to receive a solution; a loading chamber, wherein the access port is located in the loading chamber; and a plurality of culture chambers, wherein the culture chambers are radiated from the loading chamber so that the solution injected into the loading chamber through the access port is distributed into the plurality of culture chambers, wherein the plurality of culture chambers are open to an external environment and comprises a protruding edge at an opening of the plurality of culture chambers.

Embodiment 2. The device of Embodiment 1, wherein the device comprises poly(dimethyl siloxane).

Embodiment 3. The device of Embodiment 1 or 2, wherein the device is optically transparent.

Embodiment 4. The device of any one of Embodiments 1-3, wherein the access port is located in a center of the loading chamber.

Embodiment 5. The device of Embodiment 4, wherein the plurality of culture chambers are symmetrical with respect to rotations about the access port.

Embodiment 6. The device of Embodiment 5, wherein the solution injected into the loading chamber through the access port is evenly distributed into the plurality of culture chambers.

Embodiment 7. The device of any one of Embodiments 1-6, wherein the device is configured to contact a culture media from the external environment through the opening of the plurality of culture chambers.

Embodiment 8. The device of Embodiment 1, wherein the solution is a hydrogel solution.

Embodiment 9. The device of Embodiment 1, wherein the hydrogel solution comprises cells or organoids.

Embodiment 10. The device of Embodiment 1, wherein the organoids are human organoids.

Embodiment 11. The device of any one of Embodiments 1-10, wherein each of the culture chambers has a width or a height ranging from about 100 μm to about 5 cm.

Embodiment 12. The device of Embodiment 11, wherein each of the culture chambers has a width and a height of about 1 cm.

Embodiment 13. The device of any one of Embodiments 1-12, wherein at least about 80% of the organoids in the culture chamber are viable at day 21 of culturing.

Embodiment 14. The device of any one of Embodiments 1-13, wherein the protruding edge is configured to pin a meniscus of the solution at the opening of the culture chambers, allowing filling of the culture chambers without spillage of the solution through the opening.

Embodiment 15. The device of any one of Embodiments 1-14, wherein each culture chamber comprises a different type of cells or organoids for co-culturing.

Embodiment 16. The device of any one of Embodiments 1-15, wherein growth of the organoids continues for at least about 21 days.

Embodiment 17. The device of any one of Embodiments 1-16, wherein a size of the organoids increases for at least about 21 days.

Embodiment 18. The device of Embodiment 17, wherein the device decreases variability in the size of the organoids.

Embodiment 19. A method for culturing organoids, comprising: injecting a solution including cells or organoids into a loading chamber through an access port; filling a plurality of culture chambers with the solution including cells or organoids, wherein the culture chambers are radiated from the loading chamber so that the solution injected into the loading chamber is distributed into the plurality of culture chambers, wherein the plurality of culture chambers are open to an external environment and comprises a protruding edge at an opening of the culture chambers for preventing spillage of the solution through the opening; and providing a culture media to the device through the opening of the plurality of culture chambers.

Embodiment 20. The method of Embodiment 19, wherein the access port is located in a center of the loading chamber.

Embodiment 21. The method of Embodiment 20, wherein the plurality of culture chambers are symmetrical with respect to rotations about the access port.

Embodiment 22. The method of Embodiment 21, wherein the solution injected into the loading chamber through the access port is evenly distributed into the plurality of culture chambers.

Embodiment 23. The method of any one of Embodiments 19-22, wherein the solution is a hydrogel solution.

Embodiment 24. The method of any one of Embodiments 19-23, wherein the organoids are human organoids.

Embodiment 25. The method of Embodiment 23 or 24, wherein the hydrogel solution is solidified to form a hydrogel in the plurality of culture chambers after being injected into the loading chamber and distributed into the plurality of culture chambers.

Embodiment 26. The method of any one of Embodiments 19-25, wherein at least about 80% of the organoids in the culture chamber are viable at day 21 of culturing.

Embodiment 27. The method of any one of Embodiments 19-26, wherein each culture chamber comprises a different type of cells or organoids for co-culturing.

Embodiment 28. The method of any one of Embodiments 19-27, wherein growth of the organoids continues for at least about 21 days.

Embodiment 29. The method of any one of Embodiments 19-28, wherein a size of the organoids increases for at least about 21 days.

Embodiment 30. The method of Embodiment 17, wherein the device decreases variability in the size of the organoids.

Embodiment 31. The method of any one of Embodiments 19-30, wherein the culture media comprises soluble factors.

Embodiment 32. The method of Embodiment 31, wherein the soluble factors are selected from the group consisting of a growth factor, an active agent, and a combination thereof.

Embodiment 33. The method of any one of Embodiments 19-32, further comprising maturing the organoids.

Embodiment 34. The method of any one of Embodiments 19-33, further comprising assessing viability and maturation of the organoids in the plurality of culture chambers.

All patents, patent applications, publications, product descriptions, and protocols, cited in this specification are hereby incorporated by reference in their entireties. In case of a conflict in terminology, the present disclosure controls.

While it will become apparent that the subject matter herein described is well calculated to achieve the benefits and advantages set forth above, the presently disclosed subject matter is not to be limited in scope by the specific embodiments described herein. It will be appreciated that the disclosed subject matter is susceptible to modification, variation, and change without departing from the spirit thereof. Those skilled in the art will recognize or be able to ascertain using no more than routine experimentation, many equivalents to the specific embodiments described herein. Such equivalents are intended to be encompassed by the following claims.

What is claimed:

1. A device for culturing organoids, comprising
an access port configured to receive a solution;
a loading chamber, wherein the access port is located in the loading chamber; and
a plurality of culture chambers, wherein the culture chambers are radiated from the loading chamber so that the solution injected into the loading chamber through the access port is distributed into the plurality of culture chambers, wherein the plurality of culture chambers are open to an external environment and comprises a protruding edge at an opening of the plurality of culture chambers.

2. The device of claim 1, wherein the device comprises poly(dimethylsiloxane).

3. The device of claim 1, wherein the device is optically transparent.

4. The device of claim 1, wherein the access port is located in a center of the loading chamber.

5. The device of claim 4, wherein the plurality of culture chambers are symmetrical with respect to rotations about the access port.

6. The device of claim 5, wherein the solution injected into the loading chamber through the access port is evenly distributed into the plurality of culture chambers.

7. The device of claim 1, wherein the device is configured to contact a culture media from the external environment through the opening of the plurality of culture chambers.

8. The device of claim 1, wherein the solution is a hydrogel solution.

9. The device of claim 1, wherein the hydrogel solution comprises cells or organoids.

10. The device of claim 1, wherein the organoids are human organoids.

11. The device of claim 1, wherein each of the culture chambers has a width or a height ranging from about 100 μm to about 5 cm.

12. The device of claim 11, wherein each of the culture chambers has a width and a height of about 1 cm.

13. The device of claim 1, wherein at least about 80% of the organoids in the culture chamber are viable at day 21 of culturing.

14. The device of claim 1, wherein the protruding edge is configured to pin a meniscus of the solution at the opening of the culture chambers, allowing filling of the culture chambers without spillage of the solution through the opening.

15. The device of claim 1, wherein each culture chamber comprises a different type of cells or organoids for co-culturing.

16. The device of claim 1, wherein growth of the organoids continues for at least about 21 days.

17. The device of claim 1, wherein a size of the organoids increases for at least about 21 days.

18. The device of claim 17, wherein the device decreases variability in the size of the organoids.

19. A method for culturing organoids, comprising:
injecting a solution including cells or organoids into a loading chamber through an access port;
filling a plurality of culture chambers with the solution including cells or organoids, wherein the culture chambers are radiated from the loading chamber so that the solution injected into the loading chamber is distributed into the plurality of culture chambers, wherein the plurality of culture chambers are open to an external environment and comprises a protruding edge at an opening of the culture chambers for preventing spillage of the solution through the opening; and
providing a culture media to the device through the opening of the plurality of culture chambers.

20. The method of claim 19, wherein the access port is located in a center of the loading chamber.

21. The method of claim 20, wherein the plurality of culture chambers are symmetrical with respect to rotations about the access port.

22. The method of claim 21, wherein the solution injected into the loading chamber through the access port is evenly distributed into the plurality of culture chambers.

23. The method of claim 19, wherein the solution is a hydrogel solution.

24. The method claim 19, wherein the organoids are human organoids.

25. The method of claim 23, wherein the hydrogel solution is solidified to form a hydrogel in the plurality of culture chambers after being injected into the loading chamber and distributed into the plurality of culture chambers.

26. The method of claim 19, wherein at least about 80% of the organoids in the culture chamber are viable at day 21 of culturing.

27. The method of claim 19, wherein each culture chamber comprises a different type of cells or organoids for co-culturing.

28. The method of claim 19, wherein growth of the organoids continues for at least about 21 days.

29. The method of claim 19, wherein a size of the organoids increases for at least about 21 days.

30. The method of claim 17, wherein the device decreases variability in the size of the organoids.

31. The method of claim 19, wherein the culture media comprises soluble factors.

32. The method of claim 31, wherein the soluble factors are selected from the group consisting of a growth factor, an active agent, and a combination thereof.

33. The method of claim 19, further comprising maturing the organoids.

34. The method of claim 19, further comprising assessing viability and maturation of the organoids in the plurality of culture chambers.
